(19) United States
(12) Patent Application Publication
(10) Pub. No.: US 2024/0282410 A1
(43) Pub. Date: Aug. 22, 2024

(54) METHODS FOR PREDICTING IMMUNE CHECKPOINT BLOCKADE EFFICACY ACROSS MULTIPLE CANCER TYPES

(71) Applicants: Memorial Sloan-Kettering Cancer Center, New York, NY (US); Memorial Hospital for Cancer and Allied Diseases, New York, NY (US); Sloan-Kettering Institute for Cancer Research, New York, NY (US); Cleveland Clinic Foundation, Cleveland, OH (US)

(72) Inventors: Luc Morris, New York, NY (US); Timothy Chan, Cleveland, OH (US); Diego Chowell, New York, NY (US); Cristina Valero, New York, NY (US); Nils Weinhold, New York, NY (US); Seong-Keun Yoo, Cleveland, OH (US)

(21) Appl. No.: 18/571,085

(22) PCT Filed: Jun. 17, 2022
(86) PCT No.: PCT/US22/33978

§ 371 Date: Dec. 15, 2023

Related U.S. Application Data
(60) Provisional application No. 63/212,219, filed on Jun. 18, 2021.

(51) Int. Cl. G16B 40/20; G16B 20/00; G16H 20/17; G16H 50/70

(52) U.S. Cl. CPC G16B 40/20; G16B 20/00; G16H 20/17; G16H 50/70

(57) ABSTRACT

The present disclosure relates generally to methods, devices, and systems for accurately predicting the efficacy of immune checkpoint blockade therapy across multiple cancer types.

### CROSS-REFERENCE TO RELATED APPLICATIONS

This application claims the benefit of and priority to U.S. Provisional Patent Application No. 63/212,219 filed Jun. 18, 2021, the entire contents of which are incorporated herein by reference.

### TECHNICAL FIELD

The present technology relates generally to methods, devices, and systems for accurately predicting the efficacy of immune checkpoint blockade therapy across multiple cancer types.

### BACKGROUND

The following description of the background of the present technology is provided simply as an aid in understanding the present technology and is not admitted to describe or constitute prior art to the present technology.

Immune checkpoint blockade (ICB) has saved the lives of many patients with lethal cancers^{1,2}. The primary approved agents include antibodies that target CTLA-4 or PD-1/PD-L1, which can induce durable responses in patients with advanced-stage cancers. However, the majority of patients incur costs of treatment without durable benefits^{1,3}. Some large phase 3 clinical trials have reported negative results in unselected patients⁶⁻⁹, highlighting the need to identify who will respond to immunotherapy. Recent studies have described different biological factors that affect ICB efficacy^{1,2,4,10}. However, none of these factors act in isolation, and thus, cannot alone optimally identify patients who benefit from ICB across different cancer types².

Accordingly, there is an urgent need for accurate methods for pre-therapy identification of patients whose tumors will respond to ICB.

### SUMMARY OF THE PRESENT TECHNOLOGY

In one aspect, the present disclosure provides a method of training a machine learning classifier for predicting responsiveness of cancer patients to an immune checkpoint blockade (ICB) therapy, the method comprising: receiving data on a cohort of subjects, the subjects in the cohort having a plurality of cancer types; generating a training dataset based on the received data, the training dataset comprising a plurality of features for each subject in the cohort, the plurality of features comprising at least one of a blood albumin level, a blood hemoglobin level, or a blood platelet level; and applying a machine learning method to the training dataset to develop the machine learning classifier for predicting responsiveness of cancer patients to the ICB therapy, wherein applying the machine learning method comprises: applying a machine learning technique to the training dataset to each cancer type individually; performing hyperparameter optimization to identify one or more machine learning models with an accuracy that exceeds an accuracy threshold for the classifier; and determining an optimal operating-point threshold based on optimization of sensitivity and specificity of the receiver operating characteristic (ROC) curves for the training dataset; and wherein the classifier is configured to receive the plurality of features for cancer patients and generate predictors for responsiveness of the cancer patients to the ICB therapy. In some embodiments, the machine learning technique is a random forest technique, and the one or more machine learning models are random forest models. The ICB therapy may be a PD-1/PD-L1 inhibitor, a CTLA-4 inhibitor, or a combination thereof. In some embodiments, the ICB therapy comprises one or more of pembrolizumab, nivolumab, cemiplimab, atezolizumab, avelumab, durvalumab, ipilimumab, tremelimumab, ticlimumab, JTX-4014, Spartalizumab (PDR001), Camrelizumab (SHR1210), Sintilimab (IBI308), Tislelizumab (BGB-A317), Toripalimab (JS 001), Dostarlimab (TSR-042, WBP-285), INCMGA00012 (MGA012), AMP-224, AMP-514, KN035, CK-301, AUNP12, CA-170, or BMS-986189. Additionally or alternatively, in some embodiments, the predictors comprise response probability values.

In any and all embodiments of the methods disclosed herein, the plurality of features for each subject in the cohort are determined by assaying blood and/or sequencing tumor DNA.

Additionally or alternatively, in some embodiments, the machine learning classifier is an ensemble learning random forest classifier. In certain embodiments, the machine learning classifier is cancer-type specific, and the plurality of features comprises a cancer type.

Additionally or alternatively, in some embodiments, hyperparameter optimization is performed for each cancer type in the plurality of cancer types. In certain embodiments, performing the hyperparameter optimization comprises performing an exhaustive grid search technique.

In any and all embodiments of the methods disclosed herein, the plurality of features comprises a plurality of at least one genomic feature, at least one molecular feature, at least one clinical feature, and/or at least one demographic feature. In certain embodiments, the plurality of features comprises at least one genomic feature, at least one molecular feature, at least one clinical feature, and at least one demographic feature. In some embodiments, the plurality of features further comprises a plurality of: (i) a tumor mutation burden (TMB) metric, (ii) a fraction of copy-number alteration (FCNA) metric, (iii) an HLA-I evolutionary divergence (HED) metric, (iv) a loss of heterozygosity (LOH) status in HLA-I, (v) a microsatellite instability (MSI) status, (vi) a body mass index (BMI), (vii) a gender, (viii) a blood neutrophil-to-lymphocyte ratio (NLR) metric, (ix) a tumor stage, (x) an immune checkpoint inhibitor, (xi) an age, (xii) a cancer type, and/or (xiii) an indication of whether chemotherapy has been administered. In other embodiments, the plurality of features comprises: (i) a tumor mutation burden (TMB) metric, (ii) a fraction of copy-number alteration (FCNA) metric, (iii) an HLA-I evolutionary divergence (HED) metric, (iv) a loss of heterozygosity (LOH) status in HLA-I, (v) a microsatellite instability (MSI) status, (vi) a body mass index (BMI), (vii) a gender, (viii) a blood neutrophil-to-lymphocyte ratio (NLR) metric, (ix) a tumor stage, (x) an immune checkpoint inhibitor, (xi) an age, (xii) a cancer type, (xiii) an indication of whether chemotherapy has been administered, (xiv) the blood albumin level, (xv) the blood hemoglobin level, and (xvi) the blood platelet level. The tumor stage may stage I, stage II, Stage III or stage IV.

Additionally or alternatively, in some embodiments, the plurality of features comprises one or more features having discrete values and/or one or more features having continuous values. In some embodiments, the one or more features having discrete values are selected from among an indication of whether chemotherapy has been administered, cancer type, LOH in HLA-I, an immune checkpoint inhibitor, MSI status, and tumor stage. In certain embodiments, the one or more features having continuous values are selected from among a TMB metric, blood albumin level, blood NLR metric, age, blood hemoglobin level, blood platelet level, a FCNA metric, BMI, and a HED metric.

In any of the preceding embodiments, the method further comprises applying the classifier to data on a cancer patient to generate a predictor, and determining whether the cancer patient is predicted to be responsive to the ICB therapy based on the predictor and the operating-point threshold. In some embodiments, the predictor comprises a response probability value.

Additionally or alternatively, in some embodiments, the method further comprises administering an effective amount of the ICB therapy to the cancer patient predicted to be responsive to the ICB therapy based on the predictor and the operating-point threshold. In any and all embodiments of the methods disclosed herein, the cancer patient predicted to be responsive to the ICB therapy based on the predictor and the operating-point threshold exhibits extended overall survival and/or progression-free survival compared to a cancer patient that is predicted to be non-responsive to the ICB therapy based on the predictor and the operating-point threshold.

In any and all embodiments of the methods disclosed herein, the plurality of cancer types are selected from the group consisting of non-small cell lung cancers (NSCLC), small cell lung cancers (SCLC), melanoma, renal cell carcinoma, bladder cancer, head and neck cancer, sarcoma, endometrial cancer, gastric cancer, hepatobiliary cancer, colorectal cancer, esophageal cancer, pancreatic cancer, mesothelioma, ovarian cancer, and breast cancer.

In one aspect, the present disclosure provides a method of predicting responsiveness of a cancer patient to an immune checkpoint blockade (ICB) therapy using a machine learning classifier, the method comprising: receiving patient data corresponding to a plurality of features for the cancer patient; applying the machine learning classifier to the patient data to generate a predictor; and determining whether the cancer patient is predicted to be responsive to the ICB therapy based on the predictor and an operating-point threshold, wherein the machine learning classifier is trained by: receiving cohort data on a cohort of subjects, the subjects in the cohort having a plurality of cancer types; generating a training dataset based on the received cohort data, the training dataset comprising the plurality of features for each subject in the cohort, the plurality of features comprising at least one of a blood albumin level, a blood hemoglobin level, or a blood platelet level; and applying a machine learning method to the training dataset to develop the machine learning classifier for predicting responsiveness of cancer patients to the ICB therapy, wherein applying the machine learning method comprises: applying a machine learning technique to the training dataset to each cancer type individually; performing hyperparameter optimization to identify one or more machine learning models with an accuracy that exceeds an accuracy threshold for the machine learning classifier; and determining the optimal operating-point threshold based on optimization of sensitivity and specificity of the receiver operating characteristic (ROC) curves for the training dataset; and wherein the machine learning classifier is configured to receive the plurality of features for cancer patients and generate predictors for responsiveness of the cancer patients to the ICB therapy. The ICB therapy may be a PD-1/PD-L1 inhibitor, a CTLA-4 inhibitor, or a combination thereof. In some embodiments, the ICB therapy comprises one or more of pembrolizumab, nivolumab, cemiplimab, atezolizumab, avelumab, durvalumab, ipilimumab, tremelimumab, ticlimumab, JTX-4014, Spartalizumab (PDR001), Camrelizumab (SHR1210), Sintilimab (IBI308), Tislelizumab (BGB-A317), Toripalimab (JS 001), Dostarlimab (TSR-042, WBP-285), INCMGA00012 (MGA012), AMP-224, AMP-514, KN035, CK-301, AUNP12, CA-170, or BMS-986189. Additionally or alternatively, in some embodiments, the predictors comprise response probability values. In certain embodiments, the machine learning technique is a random forest technique, and the one or more machine learning models are random forest models.

Additionally or alternatively, in some embodiments, the method further comprises administering an effective amount of the ICB therapy to the cancer patient predicted to be responsive to the ICB therapy based on the predictor and the operating-point threshold. In any and all embodiments of the methods disclosed herein, the cancer patient predicted to be responsive to the ICB therapy based on the predictor and the operating-point threshold exhibits extended overall survival and/or progression-free survival compared to a cancer patient that is predicted to be non-responsive to the ICB therapy based on the predictor and the operating-point threshold. The predictor may comprise a response probability value.

Additionally or alternatively, in some embodiments, the machine learning classifier is an ensemble learning random forest classifier. In certain embodiments, the machine learning classifier is cancer-type specific, and the plurality of features comprises a cancer type.

Additionally or alternatively, in some embodiments, hyperparameter optimization is performed for each cancer type in the plurality of cancer types. In certain embodiments, performing the hyperparameter optimization comprises performing an exhaustive grid search technique.

In any and all embodiments of the methods disclosed herein, the plurality of features comprises a plurality of at least one genomic feature, at least one molecular feature, at least one clinical feature, and/or at least one demographic feature. In certain embodiments, the plurality of features comprises at least one genomic feature, at least one molecular feature, at least one clinical feature, and at least one demographic feature. In some embodiments, the plurality of features further comprises a plurality of: (i) a tumor mutation burden (TMB) metric, (ii) a fraction of copy-number alteration (FCNA) metric, (iii) an HLA-I evolutionary divergence (HED) metric, (iv) a loss of heterozygosity (LOH) status in HLA-I, (v) a microsatellite instability (MSI) status, (vi) a body mass index (BMI), (vii) a gender, (viii) a blood neutrophil-to-lymphocyte ratio (NLR) metric, (ix) a tumor stage, (x) an immune checkpoint inhibitor, (xi) an age, (xii) a cancer type, and/or (xiii) an indication of whether chemotherapy has been administered. In other embodiments, the plurality of features comprises: (i) a tumor mutation burden (TMB) metric, (ii) a fraction of copy-number alteration (FCNA) metric, (iii) an HLA-I evolutionary divergence (HED) metric, (iv) a loss of heterozygosity (LOH) status in HLA-I, (v) a microsatellite instability (MSI) status, (vi) a body mass index (BMI), (vii) a gender, (viii) a blood neutrophil-to-lymphocyte ratio (NLR) metric, (ix) a tumor stage, (x) an immune checkpoint inhibitor, (xi) an age, (xii) a cancer type, (xiii) an indication of whether chemotherapy has been administered, (xiv) the blood albumin level, (xv) the blood hemoglobin level, and (xvi) the blood platelet level. The tumor stage may stage I, stage II, Stage III or stage IV.

Additionally or alternatively, in some embodiments, the plurality of features comprises one or more features having discrete values and/or one or more features having continuous values. In some embodiments, the one or more features having discrete values are selected from among an indication of whether chemotherapy has been administered, cancer type, LOH in HLA-I, an immune checkpoint inhibitor, MSI status, and tumor stage. In certain embodiments, the one or more features having continuous values are selected from among a TMB metric, blood albumin level, blood NLR metric, age, blood hemoglobin level, blood platelet level, a FCNA metric, BMI, and a HED metric.

In any and all embodiments of the methods disclosed herein, the plurality of cancer types are selected from the group consisting of non-small cell lung cancers (NSCLC), small cell lung cancers (SCLC), melanoma, renal cell carcinoma, bladder cancer, head and neck cancer, sarcoma, endometrial cancer, gastric cancer, hepatobiliary cancer, colorectal cancer, esophageal cancer, pancreatic cancer, mesothelioma, ovarian cancer, and breast cancer.

In any and all embodiments of the methods disclosed herein, the plurality of features for each subject in the cohort are determined by assaying blood and/or sequencing tumor DNA. In certain embodiments, the plurality of features for the cancer patient are determined by assaying blood and/or sequencing tumor DNA.

In another aspect, the present disclosure provides a machine learning system for training a machine learning classifier for predicting responsiveness of cancer patients to an immune checkpoint blockade (ICB) therapy, the system comprising a processor and a memory with instructions which, when executed by the processor, cause the processor to: receive data on a cohort of subjects, the subjects in the cohort having a plurality of cancer types; generate a training dataset based on the received data, the training dataset comprising a plurality of features for each subject in the cohort, the plurality of features comprising at least one of a blood albumin level, a blood hemoglobin level, or a blood platelet level; and apply a machine learning method to the training dataset to develop the machine learning classifier for predicting responsiveness of cancer patients to the ICB therapy; wherein applying the machine learning method comprises: applying a machine learning technique to the training dataset to each cancer type individually; performing hyperparameter optimization to identify one or more machine learning models with an accuracy that exceeds an accuracy threshold for the machine learning classifier; and determining an optimal operating-point threshold based on optimization of sensitivity and specificity of the receiver operating characteristic (ROC) curves for the training dataset; and wherein the machine learning classifier is configured to receive the plurality of features for cancer patients and generate predictors for responsiveness of the cancer patients to the ICB therapy. In some embodiments, the machine learning technique is a random forest technique, and the one or more machine learning models are random forest models. The ICB therapy may be a PD-1/PD-L1 inhibitor, a CTLA-4 inhibitor, or a combination thereof. In some embodiments, the ICB therapy comprises one or more of pembrolizumab, nivolumab, cemiplimab, atezolizumab, avelumab, durvalumab, ipilimumab, tremelimumab, ticlimumab, JTX-4014, Spartalizumab (PDR001), Camrelizumab (SHR1210), Sintilimab (IBI308), Tislelizumab (BGB-A317), Toripalimab (JS 001), Dostarlimab (TSR-042, WBP-285), INCMGA00012 (MGA012), AMP-224, AMP-514, KN035, CK-301, AUNP12, CA-170, or BMS-986189.

Additionally or alternatively, in some embodiments, the machine learning classifier is an ensemble learning random forest classifier. In certain embodiments, the machine learning classifier is cancer-type specific, and the plurality of features comprises a cancer type.

Additionally or alternatively, in some embodiments, hyperparameter optimization is performed for each cancer type in the plurality of cancer types. In certain embodiments, performing the hyperparameter optimization comprises performing an exhaustive grid search technique.

In any and all embodiments of the machine learning systems disclosed herein, the plurality of features comprises a plurality of at least one genomic feature, at least one molecular feature, at least one clinical feature, and/or at least one demographic feature. In certain embodiments, the plurality of features comprises at least one genomic feature, at least one molecular feature, at least one clinical feature, and at least one demographic feature. In some embodiments, the plurality of features further comprises a plurality of: (i) a tumor mutation burden (TMB) metric, (ii) a fraction of copy-number alteration (FCNA) metric, (iii) an HLA-I evolutionary divergence (HED) metric, (iv) a loss of heterozygosity (LOH) status in HLA-I, (v) a microsatellite instability (MSI) status, (vi) a body mass index (BMI), (vii) a gender, (viii) a blood neutrophil-to-lymphocyte ratio (NLR) metric, (ix) a tumor stage, (x) an immune checkpoint inhibitor, (xi) an age, (xii) a cancer type, and/or (xiii) an indication of whether chemotherapy has been administered. In other embodiments, the plurality of features comprises: (i) a tumor mutation burden (TMB) metric, (ii) a fraction of copy-number alteration (FCNA) metric, (iii) an HLA-I evolutionary divergence (HED) metric, (iv) a loss of heterozygosity (LOH) status in HLA-I, (v) a microsatellite instability (MSI) status, (vi) a body mass index (BMI), (vii) a gender, (viii) a blood neutrophil-to-lymphocyte ratio (NLR) metric, (ix) a tumor stage, (x) an immune checkpoint inhibitor, (xi) an age, (xii) a cancer type, (xiii) an indication of whether chemotherapy has been administered, (xiv) the blood albumin level, (xv) the blood hemoglobin level, and (xvi) the blood platelet level. The tumor stage may stage I, stage II, Stage III or stage IV.

Additionally or alternatively, in some embodiments, the plurality of features comprises one or more features having discrete values and/or one or more features having continuous values. In some embodiments, the one or more features having discrete values are selected from among an indication of whether chemotherapy has been administered, cancer type, LOH in HLA-I, an immune checkpoint inhibitor, MSI status, and tumor stage. In certain embodiments, the one or more features having continuous values are selected from among a TMB metric, blood albumin level, blood NLR metric, age, blood hemoglobin level, blood platelet level, a FCNA metric, BMI, and a HED metric.

Additionally or alternatively, in some embodiments, the instructions further cause the processor to apply the machine learning classifier to data on a cancer patient to generate a predictor, and determine whether the cancer patient is predicted to be responsive to the ICB therapy based on the predictor and the operating-point threshold. The predictor may comprise a response probability value.

In any and all embodiments of the machine learning systems disclosed herein, the plurality of cancer types are selected from the group consisting of non-small cell lung cancers (NSCLC), small cell lung cancers (SCLC), melanoma, renal cell carcinoma, bladder cancer, head and neck cancer, sarcoma, endometrial cancer, gastric cancer, hepatobiliary cancer, colorectal cancer, esophageal cancer, pancreatic cancer, mesothelioma, ovarian cancer, and breast cancer.

In another aspect, the present disclosure provides a computing system for predicting responsiveness of a cancer patient to an immune checkpoint blockade (ICB) therapy, the computing system comprising a processor and a memory with instructions which, when executed by the processor, cause the processor to: receive patient data corresponding to a plurality of features for the cancer patient; apply a machine learning classifier to the patient data to generate a predictor; and determine whether the cancer patient is predicted to be responsive to the ICB therapy based on the predictor and an operating-point threshold, wherein the classifier is trained by: receiving cohort data on a cohort of subjects, the subjects in the cohort having a plurality of cancer types; generating a training dataset based on the received cohort data, the training dataset comprising the plurality of features for each subject in the cohort, the plurality of features comprising at least one of a blood albumin level, a blood hemoglobin level, or a blood platelet level; and applying a machine learning method to the training dataset to develop the machine learning classifier for predicting responsiveness of cancer patients to the ICB therapy, wherein applying the machine learning method comprises: applying a machine learning technique to the training dataset to each cancer type individually; performing hyperparameter optimization to identify one or more machine learning models with an accuracy that exceeds an accuracy threshold for the machine learning classifier; and determining the optimal operating-point threshold based on optimization of sensitivity and specificity of the receiver operating characteristic (ROC) curves for the training dataset; and wherein the machine learning classifier is configured to receive the plurality of features for cancer patients and generate predictors for responsiveness of the cancer patients to the ICB therapy. The predictor may comprise a response probability value. The ICB therapy may be a PD-1/PD-L1 inhibitor, a CTLA-4 inhibitor, or a combination thereof. In some embodiments, the ICB therapy comprises one or more of pembrolizumab, nivolumab, cemiplimab, atezolizumab, avelumab, durvalumab, ipilimumab, tremelimumab, ticlimumab, JTX-4014, Spartalizumab (PDR001), Camrelizumab (SHR1210), Sintilimab (IBI308), Tislelizumab (BGB-A317), Toripalimab (JS 001), Dostarlimab (TSR-042, WBP-285), INCMGA00012 (MGA012), AMP-224, AMP-514, KN035, CK-301, AUNP12, CA-170, or BMS-986189. Additionally or alternatively, in some embodiments, the machine learning technique is a random forest technique, and the one or more machine learning models are random forest models.

Additionally or alternatively, in some embodiments, the machine learning classifier is an ensemble learning random forest classifier. In certain embodiments, the machine learning classifier is cancer-type specific, and the plurality of features comprises a cancer type.

Additionally or alternatively, in some embodiments, hyperparameter optimization is performed for each cancer type in the plurality of cancer types. In certain embodiments, performing the hyperparameter optimization comprises performing an exhaustive grid search technique.

In any and all embodiments of the computing systems disclosed herein, the plurality of features comprises a plurality of at least one genomic feature, at least one molecular feature, at least one clinical feature, and/or at least one demographic feature. In certain embodiments, the plurality of features comprises at least one genomic feature, at least one molecular feature, at least one clinical feature, and at least one demographic feature. In some embodiments, the plurality of features further comprises a plurality of: (i) a tumor mutation burden (TMB) metric, (ii) a fraction of copy-number alteration (FCNA) metric, (iii) an HLA-I evolutionary divergence (HED) metric, (iv) a loss of heterozygosity (LOH) status in HLA-I, (v) a microsatellite instability (MSI) status, (vi) a body mass index (BMI), (vii) a gender, (viii) a blood neutrophil-to-lymphocyte ratio (NLR) metric, (ix) a tumor stage, (x) an immune checkpoint inhibitor, (xi) an age, (xii) a cancer type, and/or (xiii) an indication of whether chemotherapy has been administered. In other embodiments, the plurality of features comprises: (i) a tumor mutation burden (TMB) metric, (ii) a fraction of copy-number alteration (FCNA) metric, (iii) an HLA-I evolutionary divergence (HED) metric, (iv) a loss of heterozygosity (LOH) status in HLA-I, (v) a microsatellite instability (MSI) status, (vi) a body mass index (BMI), (vii) a gender, (viii) a blood neutrophil-to-lymphocyte ratio (NLR) metric, (ix) a tumor stage, (x) an immune checkpoint inhibitor, (xi) an age, (xii) a cancer type, (xiii) an indication of whether chemotherapy has been administered, (xiv) the blood albumin level, (xv) the blood hemoglobin level, and (xvi) the blood platelet level. The tumor stage may stage I, stage II, Stage III or stage IV.

Additionally or alternatively, in some embodiments, the plurality of features comprises one or more features having discrete values and/or one or more features having continuous values. In some embodiments, the one or more features having discrete values are selected from among an indication of whether chemotherapy has been administered, cancer type, LOH in HLA-I, an immune checkpoint inhibitor, MSI status, and tumor stage. In certain embodiments, the one or more features having continuous values are selected from among a TMB metric, blood albumin level, blood NLR metric, age, blood hemoglobin level, blood platelet level, a FCNA metric, BMI, and a HED metric. Additionally or alternatively, in some embodiments, the plurality of features for the cancer patient are determined by assaying blood and/or sequencing tumor DNA.

In any and all embodiments of the computing systems disclosed herein, the plurality of cancer types are selected from the group consisting of non-small cell lung cancers (NSCLC), small cell lung cancers (SCLC), melanoma, renal cell carcinoma, bladder cancer, head and neck cancer, sarcoma, endometrial cancer, gastric cancer, hepatobiliary cancer, colorectal cancer, esophageal cancer, pancreatic cancer, mesothelioma, ovarian cancer, and breast cancer.

In one aspect, the present disclosure provides a non-transitory computer-readable storage medium comprising instructions which, when executed by a processor of a machine learning system, configure the machine learning system to train a machine learning classifier for predicting responsiveness of cancer patients to an immune checkpoint blockade (ICB) therapy, the instructions configured to cause the processor to: receive data on a cohort of subjects, the subjects in the cohort having a plurality of cancer types; generate a training dataset based on the received data, the training dataset comprising a plurality of features for each subject in the cohort, the plurality of features comprising at least one of a blood albumin level, a blood hemoglobin level, or a blood platelet level; and apply a machine learning method to the training dataset to develop the machine learning classifier for predicting responsiveness of cancer patients to the ICB therapy; wherein applying the machine learning method comprises: applying a machine learning technique to the training dataset to each cancer type individually; performing hyperparameter optimization to identify one or more machine learning models with an accuracy that exceeds an accuracy threshold for the machine learning classifier; and determining an optimal operating-point threshold based on optimization of sensitivity and specificity of the receiver operating characteristic (ROC) curves for the training dataset; and wherein the machine learning classifier is configured to receive the plurality of features for cancer patients and generate predictors for responsiveness of the cancer patients to the ICB therapy. In some embodiments, the machine learning technique is a random forest technique, and the one or more machine learning models are random forest models. The ICB therapy may be a PD-1/PD-L1 inhibitor, a CTLA-4 inhibitor, or a combination thereof. In some embodiments, the ICB therapy comprises one or more of pembrolizumab, nivolumab, cemiplimab, atezolizumab, avelumab, durvalumab, ipilimumab, tremelimumab, ticlimumab, JTX-4014, Spartalizumab (PDR001), Camrelizumab (SHR1210), Sintilimab (IBI308), Tislelizumab (BGB-A317), Toripalimab (JS 001), Dostarlimab (TSR-042, WBP-285), INCMGA00012 (MGA012), AMP-224, AMP-514, KN035, CK-301, AUNP12, CA-170, or BMS-986189.

Additionally or alternatively, in some embodiments, the machine learning classifier is an ensemble learning random forest classifier. In certain embodiments, the machine learning classifier is cancer-type specific, and the plurality of features comprises a cancer type.

Additionally or alternatively, in some embodiments, hyperparameter optimization is performed for each cancer type in the plurality of cancer types. In certain embodiments, performing the hyperparameter optimization comprises performing an exhaustive grid search technique.

In any and all embodiments of the computer-readable storage medium disclosed herein, the plurality of features comprises a plurality of at least one genomic feature, at least one molecular feature, at least one clinical feature, and/or at least one demographic feature. In certain embodiments, the plurality of features comprises at least one genomic feature, at least one molecular feature, at least one clinical feature, and at least one demographic feature. In some embodiments, the plurality of features further comprises a plurality of: (i) a tumor mutation burden (TMB) metric, (ii) a fraction of copy-number alteration (FCNA) metric, (iii) an HLA-I evolutionary divergence (HED) metric, (iv) a loss of heterozygosity (LOH) status in HLA-I, (v) a microsatellite instability (MSI) status, (vi) a body mass index (BMI), (vii) a gender, (viii) a blood neutrophil-to-lymphocyte ratio (NLR) metric, (ix) a tumor stage, (x) an immune checkpoint inhibitor, (xi) an age, (xii) a cancer type, and/or (xiii) an indication of whether chemotherapy has been administered. In other embodiments, the plurality of features comprises: (i) a tumor mutation burden (TMB) metric, (ii) a fraction of copy-number alteration (FCNA) metric, (iii) an HLA-I evolutionary divergence (HED) metric, (iv) a loss of heterozygosity (LOH) status in HLA-I, (v) a microsatellite instability (MSI) status, (vi) a body mass index (BMI), (vii) a gender, (viii) a blood neutrophil-to-lymphocyte ratio (NLR) metric, (ix) a tumor stage, (x) an immune checkpoint inhibitor, (xi) an age, (xii) a cancer type, (xiii) an indication of whether chemotherapy has been administered, (xiv) the blood albumin level, (xv) the blood hemoglobin level, and (xvi) the blood platelet level. The tumor stage may stage I, stage II, Stage III or stage IV.

Additionally or alternatively, in some embodiments, the plurality of features comprises one or more features having discrete values and/or one or more features having continuous values. In some embodiments, the one or more features having discrete values are selected from among an indication of whether chemotherapy has been administered, cancer type, LOH in HLA-I, an immune checkpoint inhibitor, MSI status, and tumor stage. In certain embodiments, the one or more features having continuous values are selected from among a TMB metric, blood albumin level, blood NLR metric, age, blood hemoglobin level, blood platelet level, a FCNA metric, BMI, and a HED metric.

In any and all embodiments of the computer-readable storage medium disclosed herein, the instructions further cause the processor to apply the classifier to data on a cancer patient to generate a predictor, and determine whether the cancer patient is predicted to be responsive to the ICB therapy based on the predictor and the operating-point threshold. The predictor may comprise a response probability value.

In any and all embodiments of the computer-readable storage medium disclosed herein, the plurality of cancer types are selected from the group consisting of non-small cell lung cancers (NSCLC), small cell lung cancers (SCLC), melanoma, renal cell carcinoma, bladder cancer, head and neck cancer, sarcoma, endometrial cancer, gastric cancer, hepatobiliary cancer, colorectal cancer, esophageal cancer, pancreatic cancer, mesothelioma, ovarian cancer, and breast cancer.

In yet another aspect, the present disclosure provides a non-transitory computer-readable storage medium comprising instructions which, when executed by a processor of a computing system, configure the computing system to system for predicting responsiveness of a cancer patient to an immune checkpoint blockade (ICB) therapy, the instructions configured to cause the processor to: receive patient data corresponding to a plurality of features for the cancer patient; apply a machine learning classifier to the patient data to generate a predictor; and determine whether the cancer patient is predicted to be responsive to the ICB therapy based on the predictor and an operating-point threshold, wherein the classifier is trained by: receiving cohort data on a cohort of subjects, the subjects in the cohort having a plurality of cancer types; generating a training dataset based on the received cohort data, the training dataset comprising the plurality of features for each subject in the cohort, the plurality of features comprising at least one of a blood albumin level, a blood hemoglobin level, or a blood platelet level; and applying a machine learning method to the training dataset to develop the machine learning classifier for predicting responsiveness of cancer patients to the ICB therapy, wherein applying the machine learning method comprises: applying a machine learning technique to the training dataset to each cancer type individually; performing hyperparameter optimization to identify one or more machine learning models with an accuracy that exceeds an accuracy threshold for the machine learning classifier; and determining the optimal operating-point threshold based on optimization of sensitivity and specificity of the receiver operating characteristic (ROC) curves for the training dataset; and wherein the machine learning classifier is configured to receive the plurality of features for cancer patients and generate predictors for responsiveness of the cancer patients to the ICB therapy. The ICB therapy may be a PD-1/PD-L1 inhibitor, a CTLA-4 inhibitor, or a combination thereof. In some embodiments, the ICB therapy comprises one or more of pembrolizumab, nivolumab, cemiplimab, atezolizumab, avelumab, durvalumab, ipilimumab, tremelimumab, ticlimumab, JTX-4014, Spartalizumab (PDR001), Camrelizumab (SHR1210), Sintilimab (IBI308), Tislelizumab (BGB-A317), Toripalimab (JS 001), Dostarlimab (TSR-042, WBP-285), INCMGA00012 (MGA012), AMP-224, AMP-514, KN035, CK-301, AUNP12, CA-170, or BMS-986189. Additionally or alternatively, in some embodiments, the predictor comprises a response probability value. In certain embodiments, the machine learning technique is a random forest technique, and the one or more machine learning models are random forest models.

Additionally or alternatively, in some embodiments, the machine learning classifier is an ensemble learning random forest classifier. In certain embodiments, the machine learning classifier is cancer-type specific, and the plurality of features comprises a cancer type.

Additionally or alternatively, in some embodiments, hyperparameter optimization is performed for each cancer type in the plurality of cancer types. In certain embodiments, performing the hyperparameter optimization comprises performing an exhaustive grid search technique.

In any and all embodiments of the computer-readable storage medium disclosed herein, the plurality of features comprises a plurality of at least one genomic feature, at least one molecular feature, at least one clinical feature, and/or at least one demographic feature. In certain embodiments, the plurality of features comprises at least one genomic feature, at least one molecular feature, at least one clinical feature, and at least one demographic feature. In some embodiments, the plurality of features further comprises a plurality of: (i) a tumor mutation burden (TMB) metric, (ii) a fraction of copy-number alteration (FCNA) metric, (iii) an HLA-I evolutionary divergence (HED) metric, (iv) a loss of heterozygosity (LOH) status in HLA-I, (v) a microsatellite instability (MSI) status, (vi) a body mass index (BMI), (vii) a gender, (viii) a blood neutrophil-to-lymphocyte ratio (NLR) metric, (ix) a tumor stage, (x) an immune checkpoint inhibitor, (xi) an age, (xii) a cancer type, and/or (xiii) an indication of whether chemotherapy has been administered. In other embodiments, the plurality of features comprises: (i) a tumor mutation burden (TMB) metric, (ii) a fraction of copy-number alteration (FCNA) metric, (iii) an HLA-I evolutionary divergence (HED) metric, (iv) a loss of heterozygosity (LOH) status in HLA-I, (v) a microsatellite instability (MSI) status, (vi) a body mass index (BMI), (vii) a gender, (viii) a blood neutrophil-to-lymphocyte ratio (NLR) metric, (ix) a tumor stage, (x) an immune checkpoint inhibitor, (xi) an age, (xii) a cancer type, (xiii) an indication of whether chemotherapy has been administered, (xiv) the blood albumin level, (xv) the blood hemoglobin level, and (xvi) the blood platelet level. The tumor stage may stage I, stage II, Stage III or stage IV.

Additionally or alternatively, in some embodiments, the plurality of features comprises one or more features having discrete values and/or one or more features having continuous values. In some embodiments, the one or more features having discrete values are selected from among an indication of whether chemotherapy has been administered, cancer type, LOH in HLA-I, an immune checkpoint inhibitor, MSI status, and tumor stage. In certain embodiments, the one or more features having continuous values are selected from among a TMB metric, blood albumin level, blood NLR metric, age, blood hemoglobin level, blood platelet level, a FCNA metric, BMI, and a HED metric. In certain embodiments, the plurality of features for the cancer patient are determined by assaying blood and/or sequencing tumor DNA.

In any and all embodiments of the computer-readable storage medium disclosed herein, the plurality of cancer types are selected from the group consisting of non-small cell lung cancers (NSCLC), small cell lung cancers (SCLC), melanoma, renal cell carcinoma, bladder cancer, head and neck cancer, sarcoma, endometrial cancer, gastric cancer, hepatobiliary cancer, colorectal cancer, esophageal cancer, pancreatic cancer, mesothelioma, ovarian cancer, and breast cancer.

### BRIEF DESCRIPTION OF THE DRAWINGS

FIGS. 1A-ID show an overview of the development of the model for integrated clinical-genetic prediction of ICB response. FIG. 1A shows a bar chart showing the number of patients in each of 16 cancer types. Responses were categorized based on RECIST v1.1 criteria (Eisenhauer, E. A. & Verweij, J., Ejc Suppl 7, 5-5 (2009)) or best radiographic response. Complete Responses (CR) and partial responses (PR) were classified as responsive (R); and stable disease (SD) and progressive disease (PD) were classified as non-responsive (NR). Numbers in parentheses denote the number of patients in NR and R, respectively. FIG. 1B shows a general overview of the random forest model training and testing procedure. Sixteen cancer types were divided into training (80%) and testing (20%) subsets individually. A random forest model was trained on multiple genomic, molecular, demographic, and clinical features on the training data using 5-fold cross-validation to predict ICB response (NR and R). The resulting trained model with the best hyperparameters was evaluated using various performance metrics using the test set. FIG. 1C shows the feature contribution of the 16 model features calculated in the training set to predict ICB response. FIG. 1D shows ROC curves and the corresponding AUC values of the RF16 model, RF11 model, and tumor mutation burden (TMB) alone in the training set across multiple cancer types. The numbers on the ROC curves denote the corresponding optimal cutpoints for RF16, which maximize the sensitivity and specificity of the response prediction.

FIGS. 2A-2H show model performance across multiple cancer types in the test set. FIG. 2A shows ROC curves and corresponding AUC values of RF16, RF11, and TMB alone. FIG. 2B shows comparison of response probability distributions calculated by RF16 between NR and R groups. FIG. 2C shows comparison of TMB between NR and R groups. P-values were calculated using the two-sided Mann-Whitney U test. FIGS. 2D-2G show confusion matrices showing predicted outcomes generated by RF16 and TMB, as indicated, (FIG. 2D) in pan-cancer, (FIG. 2E) in melanoma, (FIG. 2F) in NSCLC, and (FIG. 2G) in others (not melanoma/NSCLC), respectively. To define high TMB tumors, the threshold of ≥10 mut/Mb, which was approved by the FDA to predict ICB efficacy of solid tumors with pembrolizumab, was applied. FIG. 2H shows performance measurements of RF16 and TMB illustrated by sensitivity, specificity, accuracy, PPV, and NPV.

FIGS. 3A-3P show that the model of the present technology predicts overall survival (OS) and progression-free survival (PFS) across multiple cancer types in the test set. FIG. 3A shows a comparison of C-index and 95% CI for predicting OS among RF16, RF11, and TMB in pan-cancer cohort. FIG. 3B shows pan-cancer association between ICB response predicted by RF16 and OS. FIG. 3C shows a comparison of C-index and 95% CI for predicting OS among RF16, RF11, and TMB in melanoma. FIG. 3D shows the association between ICB response predicted by the RF16 and OS in melanoma. FIG. 3E shows a comparison of C-index and 95% CI for predicting OS among RF16, RF11, and TMB in non-small cell lung cancer (NSCLC). FIG. 3F shows the association between ICB response predicted by the RF16 and OS in NSCLC. FIG. 3G shows a comparison of C-index and 95% CI for predicting OS among RF16, RF11, and TMB in non-melanoma/non-NSCLC cancers. FIG. 3H shows the association between ICB response predicted by RF16 and OS in non-melanoma/non-NSCLC cancers. FIG. 3I shows a comparison of C-index and 95% CI for predicting PFS among RF16, RF11, and TMB in a pan-cancer cohort. FIG. 3J shows the pan-cancer association between ICB response predicted by RF16 and PFS. FIG. 3K shows a comparison of C-index and 95% CI for predicting PFS among RF16, RF11, and TMB in melanoma. FIG. 3L shows the association between ICB response predicted by RF16 and PFS in melanoma. FIG. 3M shows a comparison of C-index and 95% CI for predicting PFS among RF16, RF11, and TMB in NSCLC. FIG. 3N shows the association between ICB response predicted by RF16 and PFS in NSCLC. FIG. 3O shows a comparison of C-index and 95% CI for predicting PFS among RF16, RF11, and TMB in non-melanoma/non-NSCLC cancers. FIG. 3P shows the association between ICB response predicted by RF16 and PFS in non-melanoma/non-NSCLC cancers. P-values for comparison of C-indices and survival times were computed using the paired Student t-test and two-sided log-rank test, respectively.

FIG. 4 depicts a flow diagram showing sample collection and filtering process.

FIGS. 5A-5B show the distribution of response probabilities calculated by the integrated model in each cancer group. Density plots showing response probability values (FIG. 5A) in the training set and (FIG. 5B) in the test set. The vertical lines denote the cancer-specific group optimal cut-points identified in the training set.

FIG. 6 shows performance of the model disclosed herein illustrated by the precision-recall curve across multiple cancer types in the training set. The corresponding AUPRC values of RF16, RF11, and TMB are shown in pan-cancer and each cancer group [melanoma, NSCLC, and others (non-melanoma/non-NSCLC)].

FIG. 7 shows performance of the model disclosed herein illustrated by the precision-recall curve across multiple cancer types in the test set. The corresponding AUPRC values of RF16, RF11, and TMB are shown in pan-cancer and each cancer group [melanoma, NSCLC, and others (non-melanoma/non-NSCLC)].

FIGS. 8A-8B show a comparison of each model feature performance with that from the integrated RF16 model across multiple cancer types. AUC and AUPRC of each model feature are shown in pan-cancer and each cancer group [melanoma, NSCLC, and others (non-melanoma/non-NSCLC)] in the (FIG. 8A) training set and in the (FIG. 8B) test set. None of RF16's features alone could achieve the level of performance achieved by RF16.

FIGS. 9A-9D show model development in the training set. FIG. 9A shows confusion matrices showing suboptimal response outcomes predicted by RF16 in pan-cancer and each cancer group [melanoma, NSCLC, and others (non-melanoma/non-NSCLC)] using a single pan-cancer optimal threshold based on the ROC curve. FIG. 9B shows confusion matrices showing improved response predictions using RF16 in each cancer group using three different optimal thresholds derived from each cancer-group-specific ROC curve (FIG. 1D). FIG. 9C shows confusion matrices showing response outcomes predicted by TMB in each cancer group. To define high TMB tumors, the threshold of ≥10 mut/Mb was applied. FIG. 9D shows performance measurements of the model and TMB illustrated by sensitivity, specificity, accuracy, PPV, and NPV.

FIGS. 10A-10D show a comparison of the response probabilities computed from the RF16 model trained on pan-cancer data with those from separate models trained on cancer-specific data. Distribution of response probabilities in the (FIG. 10A) training set and in the (FIG. 10B) test set. Correlation plots of response probabilities calculated by the pan-cancer RF16 model and those calculated by separate cancer-specific models in the (FIG. 10C) training set and in the (FIG. 10D) test set.

FIGS. 11A-11D show a comparison of the predictive performance of the RF16 model trained on pan-cancer with those from separate models trained on cancer-specific data. Performance of the (FIG. 11A) pan-cancer RF16 model and the (FIG. 11B) cancer-specific model in the training set. Performance of the (FIG. 11C) pan-cancer RF16 model and the (FIG. 11D) cancer-specific model in the test set.

FIGS. 12A-12D show a comparison of the predictive performance of RF16 with a logistic regression model. Performance of the (FIG. 12A) RF16 model and the (FIG. 12B) logistic regression in the training set. Performance of the (FIG. 12C) RF16 model and the (FIG. 12D) logistic regression in the test set.

FIGS. 13A-13B show performance of the model of the present technology illustrated by the brier score and C-index in the training set. FIG. 13A shows brier score showing that RF16 for predicting OS has a smaller error compared to a reference model, based on RF11, or based on TMB alone in pan-cancer and each cancer group [melanoma, NSCLC, and others (non-melanoma/non-NSCLC)]. Reference model denotes a random model. FIG. 13B shows a comparison of C-index and 95% CI for predicting OS among RF16, RF11, and TMB. P-values were computed using the paired Student t-test.

FIG. 14 shows performance of the model of the present technology illustrated by the brier score in the test set. Brier score showing that RF16 for predicting OS has a smaller error compared to a reference model, based on RF11, or based on TMB alone in pan-cancer and each cancer group [melanoma, NSCLC, and others (non-melanoma/non-NSCLC)]. Reference model denotes a random model.

FIGS. 15A-15H show that the model of the present technology predicts overall survival across multiple cancer types in the training set. FIGS. 15A, 15C, 15E, and 15G show the association between ICB response predicted by RF16 and OS in pan-cancer and each cancer group. FIGS. 15B, 15D, 15F, and 15H show the association between high TMB and OS in pan-cancer and each cancer group. High TMB was defined as any tumor with ≥10 mut/Mb. P-values were computed using the two-sided log-rank test.

FIGS. 16A-16D show the association of high TMB and overall survival in pan-cancer and each cancer group in the test set. P-values were computed using the two-sided log-rank test. High TMB was defined as any tumor with ≥10 mut/Mb.

FIG. 17 shows that the model of the present technology predicts progression-free survival across multiple cancer types in the training set. Comparison of C-index and 95% CI for predicting PFS among RF16, RF11, and TMB in pan-cancer and each cancer group. P-values were computed using the paired Student t-test.

FIGS. 18A-18H show that the model of the present technology predicts progression-free survival across multiple cancer types in the training set. FIGS. 18A, 18C, 18E, and 18G show the association between ICB response predicted by the integrated RF16 model and PFS in pan-cancer and each cancer group. FIGS. 18B, 18D, 18F, and 18H show the association between high TMB and PFS in pan-cancer and each cancer group. High TMB was defined as any tumor with ≥10 mut/Mb. P-values were computed using the two-sided log-rank test.

FIGS. 19A-19D show the association of high TMB and progression-free survival in pan-cancer and each cancer group in the test set. P-values were computed using the two-sided log-rank test. High TMB was defined as any tumor with ≥10 mut/Mb.

FIGS. 20A-20B show the association of TMB with immunotherapy response is not confounded by the melanoma subtype. FIG. 20A shows differences in TMB between responders and non-responders across different melanoma subtypes. FIG. 20B shows logistic regression for immunotherapy response where TMB and melanoma subtype are included in the model. Results show that the effect of TMB is not affected by the melanoma subtype.

FIG. 21 shows characteristics of patients in the study. Abbreviations: NSCLC, non-small cell lung cancer; SCLC, small cell lung cancer; ICB, immune checkpoint blockade; combo, PD-1/PD-L1 plus CTLA-4.

FIG. 22 shows the effects of the features of the model disclosed herein and their corresponding p-values estimated from the logistic regression. ^{a}Melanoma was used as the reference group.

FIG. 23A is a block diagram depicting an embodiment of a network environment comprising a client device in communication with server device.

FIG. 23B is a block diagram depicting a cloud computing environment comprising client device in communication with cloud service providers.

FIGS. 23C and 23D are block diagrams depicting embodiments of computing devices useful in connection with the methods and systems described herein.

FIG. 24 depicts a system that includes a computing device and a sample processing system according to various potential embodiments.

### DETAILED DESCRIPTION

It is to be appreciated that certain aspects, modes, embodiments, variations and features of the present methods are described below in various levels of detail in order to provide a substantial understanding of the present technology. It is to be understood that the present disclosure is not limited to particular uses, methods, reagents, compounds, compositions or biological systems, which can, of course, vary. It is also to be understood that the terminology used herein is for the purpose of describing particular embodiments only, and is not intended to be limiting.

Machine learning approaches have been shown to successfully overcome limitations of predictors that rely on a single feature by combining different types of features in a nonlinear fashion^{11,12}. However, no individual biological factor can optimally identify patients who benefit from ICB across different cancer types².

The present disclosure provides a machine learning model to predict ICB response by integrating genomic, molecular, demographic, and clinical data from a cohort of 1,479 ICB-treated patients across 16 different cancer types, and assessing the individual contribution of multiple biological features to ICB response when combined in a single predictive framework. The model achieved high sensitivity and specificity in predicting clinical response to immunotherapy and predicted both overall survival and progression-free survival in the test data across different cancer types. Importantly, the model disclosed herein significantly outperformed predictions based on tumor mutation burden (TMB), which was recently approved by the Food and Drug Administration (FDA) for this purpose⁵. Additionally, the model provides quantitative assessments of the model features that are most salient for the predictions. Accordingly, the methods, devices, and systems of the present technology are useful in improving the accuracy of clinical decision-making in immunotherapy, and informing future therapeutic interventions.

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value)

As used herein, the “administration” of an agent or drug to a subject includes any route of introducing or delivering to a subject a compound to perform its intended function. Administration can be carried out by any suitable route, including but not limited to, orally, intranasally, parenterally (intravenously, intramuscularly, intraperitoneally, or subcutaneously), rectally, intrathecally, intratumorally or topically. Administration includes self-administration and the administration by another.

The terms “cancer” or “tumor” are used interchangeably and refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell. As used herein, the term “cancer” includes premalignant, as well as malignant cancers. In some embodiments, the cancer is colorectal cancer, lung cancer, breast cancer, ovarian cancer, uterine cancer, or thyroid cancer.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a compound or composition known to exhibit the desired therapeutic effect) and a negative control (a subject or a sample that does not receive the therapy or receives a placebo) are typically employed.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

The term “immune checkpoint” refers to a component of the immune system which provides inhibitory signals to its components in order to regulate immune reactions. Known immune checkpoint proteins comprise CTLA-4, PD-1 and its ligands PD-L1 and PD-L2 and in addition LAG-3, BTLA, B7H3, B7H4, TIM3, and KIR. The pathways involving LAGS, BTLA, B7H3, B7H4, TIM3, and KIR are recognized in the art to constitute immune checkpoint pathways similar to the CTLA-4 and PD-1 dependent pathways (see e.g. Pardon, 2012, Nature Rev Cancer 12:252-264; Mellman et al., 2011, Nature 480:480-489). Checkpoint proteins regulate T-cell activation or function. These proteins are responsible for co-stimulatory or inhibitory interactions of T-cell responses. Immune checkpoint proteins regulate and maintain self-tolerance and the duration and amplitude of physiological immune responses.

As used herein, the term “immune checkpoint blockade therapy” refers to therapy with molecules that totally or partially reduce, inhibit, interfere with or modulate one or more checkpoint proteins. Immune checkpoint inhibitors may include antibodies or are derived from antibodies.

“Negative predictive value (NPV)” is defined as the proportion of subjects with a negative test result who are correctly identified. A high NPV means that when the test yields a negative result, it is unlikely that the result should have been positive. The NPV is determined as:

$$NPV = \frac{\# of True Negatives}{( \# of True Negatives + \# of False Negatives )} = \frac{\# of True Negatives}{\# of Negative calls}$$

where a “true negative” is the event that the test makes a negative prediction, and the subject has a negative result under the gold standard, and a “false negative” is the event that the test makes a negative prediction, and the subject has a positive result under the gold standard.

The “positive predictive value (PPV),” or “precision rate” is a summary statistic used to describe the proportion of subjects with positive results who are correctly identified. It is a measure of the performance of a predictive method, as it reflects the probability that a positive result reflects the underlying condition being tested for. Its value does however depend on the prevalence of the outcome of interest, which may be unknown for a particular target population. The PPV can be derived using Bayes' theorem. The PPV is defined as:

$$PPV = \frac{\# of True Positives}{( \# of True Positives + \# of False Positives )} = \frac{\# of True Positives}{\# of Positive calls}$$

where a “true positive” is the event that the predictive test makes a positive prediction, and the subject has a positive result under the gold standard, and a “false positive” is the event that the test makes a positive prediction, and the subject has a negative result under the gold standard.

If the prevalence, sensitivity, and specificity are known, the positive and negative predictive values (PPV and NPV) can be calculated for any prevalence as follows:

$$PPV = \frac{sensitivity \times prevalence}{sensitivity \times prevalence + ( 1 - specificity ) \times ( 1 - prevalence )} \\ NPV = \frac{specificity \times ( 1 - prevalence )}{( 1 - sensitivity ) \times prevalence + specificity \times ( 1 - prevalence )}$$

If the prevalence of the disease is very low, the positive predictive value will not be close to 1, even if both the sensitivity and specificity are high. Thus in screening the general population it is inevitable that many people with positive test results will be false positives. The rarer the abnormality, the higher the certainty that a negative test indicates no abnormality, and the lower the certainty that a positive result truly indicates an abnormality. The prevalence can be interpreted as the probability before the test is carried out that the subject has the disease, known as the prior probability of disease. The positive and negative predictive values are the revised estimates of the same probability for those subjects who are positive and negative on the test, and are known as posterior probabilities. The difference between the prior and posterior probabilities is one way of assessing the usefulness of the test.

For any test result, one can compare the probability of obtaining that result if the patient truly had the condition of interest with the corresponding probability if he or she were healthy. The ratio of these probabilities is called the likelihood ratio, calculated as sensitivity/(1-specificity). (Altman D G, Bland J M (1994). BMJ 309 (6947):102).

In statistics, sensitivity and specificity are statistical measures of the performance of a binary classification test. “Sensitivity” (also called “recall rate”) measures the proportion of actual positives which are correctly identified as such (e.g. the percentage of subjects who are correctly identified as having a condition). Sensitivity relates to the ability of a predictive test to identify positive results and is computed as the number of true positives divided by the sum of the number of true positives and the number of false negatives. “Specificity” measures the proportion of negatives which are correctly identified (e.g., the percentage of subjects who are correctly identified as not having the condition). Specificity relates to the ability of a predictive test to identify negative results and is computed as the number of true negatives divided by the sum of the number of true negatives and the number of false positives. Sensitivity and specificity are closely related to the concepts of type I and type II errors. A theoretical, optimal prediction aims to achieve 100% sensitivity and 100% specificity, however theoretically any predictor will possess a minimum error bound known as the Bayes error rate.

For any test, there is usually a trade-off between sensitivity and specificity, which can be represented graphically using a receiver operating characteristic (ROC) curve. In some embodiments, a ROC is used to generate a summary statistic. Some common versions are: the intercept of the ROC curve with the line at 90 degrees to the no-discrimination line (also called Youden's J statistic); the area between the ROC curve and the no-discrimination line; the area under the ROC curve, or “AUC” (“Area Under Curve”), or A′ (pronounced “a-prime”); d′ (pronounced “d-prime”), the distance between the mean of the distribution of activity in the system under noise-alone conditions and its distribution under signal-alone conditions, divided by their standard deviation, under the assumption that both these distributions are normal with the same standard deviation. Under these assumptions, it can be proved that the shape of the ROC depends only on d′.

As used herein, the term “overall survival” or “OS” means the observed length of life from the start of treatment to death or the date of last contact.

As used herein, “progression free survival” or “PFS” is the time from treatment to the date of the first confirmed disease progression per RECIST 1.1 and immune-related RECIST (irRECIST) criteria.

“RECIST” shall mean an acronym that stands for “Response Evaluation Criteria in Solid Tumors” and is a set of published rules that define when cancer patients improve (“respond”), stay the same (“stable”) or worsen (“progression”) during treatments. Response as defined by RECIST criteria have been published, for example, at Journal of the National Cancer Institute, Vol. 92, No. 3, Feb. 2, 2000 and RECIST criteria can include other similar published definitions and rule sets. One skilled in the art would understand definitions that go with RECIST criteria, as used herein, such as “Partial Response (PR),” “Complete Response (CR),” “Stable Disease (SD)” and “Progressive Disease (PD).”

The irRECIST overall tumor assessment is based on total measurable tumor burden (TMTB) of measured target and new lesions, non-target lesion assessment and new non-measurable lesions. At baseline, the sum of the longest diameters (SumD) of all target lesions (up to 2 lesions per organ, up to total 5 lesions) is measured. At each subsequent tumor assessment (TA), the SumD of the target lesions and of new, measurable lesions (up to 2 new lesions per organ, total 5 new lesions) are added together to provide the TMTB.

Overall Assessments by irRECIST

| | |
|---|---|
| Complete Response (irCR) | Complete disappearance of all measurable and non- |
| | measurable lesions. Lymph nodes must decrease to <10 mm |
| | in short axis. |
| Partial Response (irPR) | Decrease of ≥30% in TMTB relative to baseline, non-target |
| | lesions are irNN, and no unequivocal progression of new |
| | nonmeasurable lesions |
| | If new measurable lesions appear in subjects with no |
| | target lesions at baseline, irPD will be assessed. That irPD |
| | time point will be considered a new baseline, and all |
| | subsequent time points will be compared to it for response |
| | assessment. irPR is possible if the TMTB of new measurable |
| | lesions decreases by ≥30% compared to the first irPD |
| | documentation |
| | irRECIST can be used in the adjuvant setting, in |
| | subjects with no visible disease on CT/MRI scans. The |
| | appearance of new measurable lesion(s) automatically leads to |
| | an increase in TMTB by 100% and leads to irPD. These |
| | subjects can achieve a response if the TMTB decreases at |
| | follow-up, as a sign of delayed response. |
| | Based on the above, sponsors may consider enrolling |
| | subjects with no measurable disease and/or no visible disease in |
| | studies with response related endpoints. |
| Stable Disease (irSD) | Failure to meet criteria for irCR or irPR in the absence of irPD |
| Progressive Disease (irPD) | Minimum 20% increase and minimum 5 mm absolute increase |
| | in TMTB compared to nadir, or irPD for non-target or new |
| | non-measurable lesions. Confirmation of progression is |
| | recommended minimum 4 weeks after the first irPD |
| | assessment. An irPD confirmation scan may be recommended |
| | for subjects with a minimal TMTB %-increase over 20% and |
| | especially during the flare time-window of the first 12 weeks of |
| | treatment, depending on the compound efficacy expectations, to |
| | account for expected delayed response. |
| | In irRECIST a substantial and unequivocal increase of |
| | non-target lesions is indicative of progression. |
| | IrPD may be assigned for a subject with multiple new |
| | non-measurable lesions if they are considered to be a sign of |
| | unequivocal massive worsening |
| Other | irNE: used in exceptional cases where insufficient data exist. |
| | irND: in adjuvant setting when no disease is detected |
| | irNN:, no target disease was identified at baseline, and at |
| | follow-up the subject fails to meet criteria for irCR or irPD |

As used herein, the terms “subject”, “patient”, or “individual” can be an individual organism, a vertebrate, a mammal, or a human. In some embodiments, the subject, patient or individual is a human.

As used herein, “survival” refers to the subject remaining alive, and includes overall survival as well as progression free survival.

As used herein, the term “therapeutic agent” is intended to mean a compound that, when present in an effective amount, produces a desired therapeutic effect on a subject in need thereof.

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

As used herein, the terms “tumor mutation burden” or “TMB” refer to the level, e.g., number, of an alteration (e.g., one or more alterations, e.g., one or more somatic alterations) per a preselected unit (e.g., per megabase) in a predetermined set of genes (e.g., in the coding regions of the predetermined set of genes) in a tumor. Tumor mutation burden can be measured, e.g., on a whole genome or exome basis, or on the basis of a subset of genome or exome. In certain embodiments, the tumor mutation burden measured on the basis of a subset of genome or exome can be extrapolated to determine a whole genome or exome mutation burden.

In certain embodiments, the tumor mutation burden is measured in a tumor sample (e.g., a tumor sample or a sample derived from a tumor), from a subject. In certain embodiments, the tumor mutation burden is expressed as a percentile, e.g., among the mutation burden in samples from a reference population. In certain embodiments, the reference population includes patients having the same type of cancer as the subject. In other embodiments, the reference population includes patients who are receiving, or have received, the same type of therapy, as the subject. In certain embodiments, the TMB correlates with the whole genome or exome mutation load.

It is also to be appreciated that the various modes of treatment of disorders as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

### Systems, Devices, and Methods for Predicting the Efficacy of Immune Checkpoint Blockade Therapy Across Multiple Cancer Types

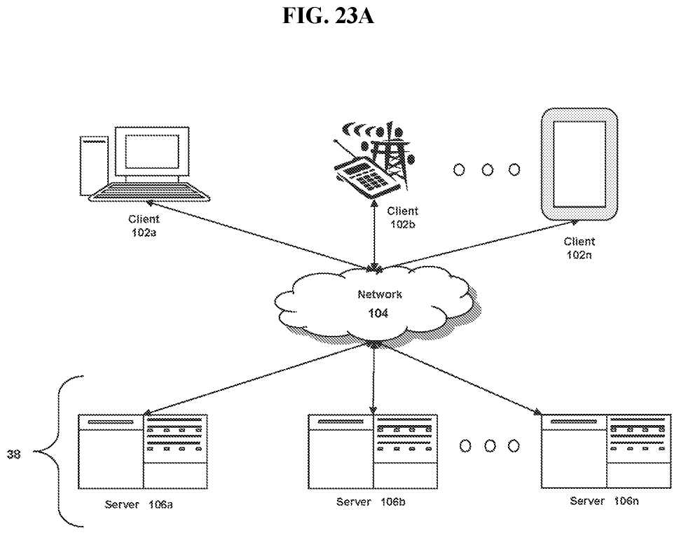

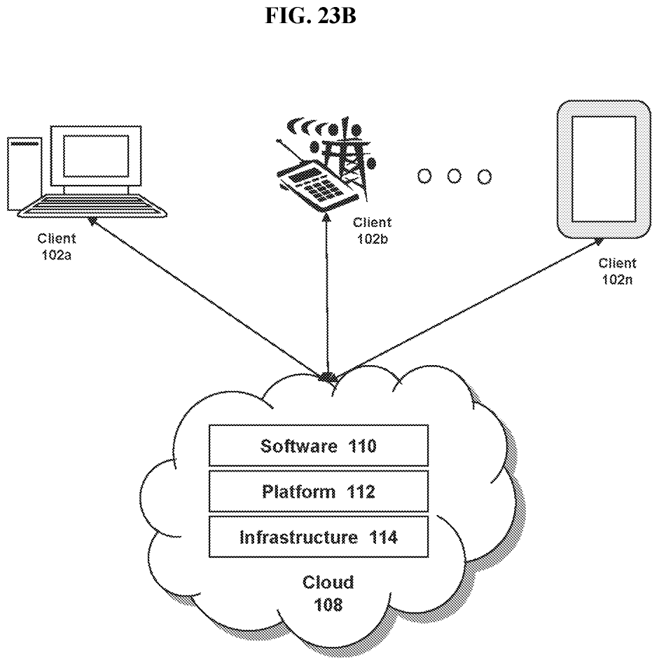

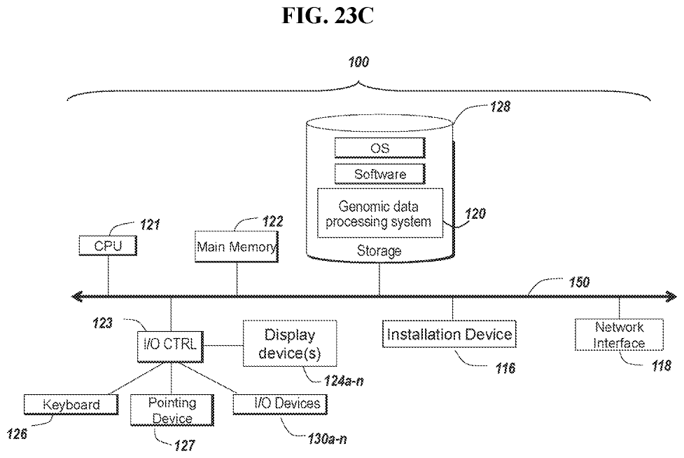

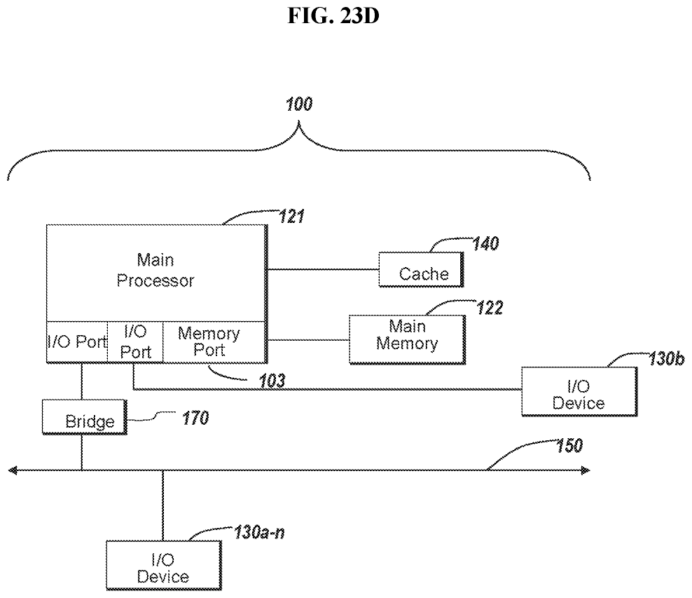

Aspects of the operating environment as well as associated system components (e.g., hardware elements) in connection with various embodiments of the methods and systems described herein will now be discussed. Referring to FIG. 23A, an embodiment of a network environment is depicted. In brief overview, the network environment includes one or more clients 102a-102n (also generally referred to as local machine(s) 102, client(s) 102, client node(s) 102, client machine(s) 102, client computer(s) 102, client device(s) 102, endpoint(s) 102, or endpoint node(s) 102) in communication with one or more servers 106a-106n (also generally referred to as server(s) 106, node 106, or remote machine(s) 106) via one or more networks 104. In some embodiments, a client 102 has the capacity to function as both a client node seeking access to resources provided by a server and as a server providing access to hosted resources for other clients 102a-102n.

Although FIG. 23A shows a network 104 between the clients 102 and the servers 106, the clients 102 and the servers 106 may be on the same network 104. In some embodiments, there are multiple networks 104 between the clients 102 and the servers 106. In one of these embodiments, a network 104′ (not shown) may be a private network and a network 104 may be a public network. In another of these embodiments, a network 104 may be a private network and a network 104′ a public network. In still another of these embodiments, networks 104 and 104′ may both be private networks.

The network 104 may be connected via wired or wireless links. Wired links may include Digital Subscriber Line (DSL), coaxial cable lines, or optical fiber lines. The wireless links may include BLUETOOTH, Wi-Fi, Worldwide Interoperability for Microwave Access (WiMAX), an infrared channel or satellite band. The wireless links may also include any cellular network standards used to communicate among mobile devices, including standards that qualify as 1G, 2G, 3G, 4G, or 5G. The network standards may qualify as one or more generation of mobile telecommunication standards by fulfilling a specification or standards such as the specifications maintained by International Telecommunication Union. The 3G standards, for example, may correspond to the International Mobile Telecommunications-2000 (IMT-2000) specification, and the 4G standards may correspond to the International Mobile Telecommunications Advanced (IMT-Advanced) specification. Examples of cellular network standards include AMPS, GSM, GPRS, UMTS, LTE, LTE Advanced, Mobile WiMAX, and WiMAX-Advanced. Cellular network standards may use various channel access methods e.g. FDMA, TDMA, CDMA, or SDMA. In some embodiments, different types of data may be transmitted via different links and standards. In other embodiments, the same types of data may be transmitted via different links and standards.

The network 104 may be any type and/or form of network. The geographical scope of the network 104 may vary widely and the network 104 can be a body area network (BAN), a personal area network (PAN), a local-area network (LAN), e.g. Intranet, a metropolitan area network (MAN), a wide area network (WAN), or the Internet. The topology of the network 104 may be of any form and may include, e.g., any of the following: point-to-point, bus, star, ring, mesh, or tree. The network 104 may be an overlay network which is virtual and sits on top of one or more layers of other networks 104′. The network 104 may be of any such network topology as known to those ordinarily skilled in the art capable of supporting the operations described herein. The network 104 may utilize different techniques and layers or stacks of protocols, including, e.g., the Ethernet protocol, the internet protocol suite (TCP/IP), the ATM (Asynchronous Transfer Mode) technique, the SONET (Synchronous Optical Networking) protocol, or the SDH (Synchronous Digital Hierarchy) protocol. The TCP/IP internet protocol suite may include application layer, transport layer, internet layer (including, e.g., IPv6), or the link layer. The network 104 may be a type of a broadcast network, a telecommunications network, a data communication network, or a computer network.

In some embodiments, the system may include multiple, logically-grouped servers 106. In one of these embodiments, the logical group of servers may be referred to as a server farm 38 or a machine farm 38. In another of these embodiments, the servers 106 may be geographically dispersed. In other embodiments, a machine farm 38 may be administered as a single entity. In still other embodiments, the machine farm 38 includes a plurality of machine farms 38. The servers 106 within each machine farm 38 can be heterogeneous—one or more of the servers 106 or machines 106 can operate according to one type of operating system platform (e.g., WINDOWS NT, manufactured by Microsoft Corp. of Redmond, Washington), while one or more of the other servers 106 can operate on according to another type of operating system platform (e.g., Unix, Linux, or Mac OS X).

In one embodiment, servers 106 in the machine farm 38 may be stored in high-density rack systems, along with associated storage systems, and located in an enterprise data center. In this embodiment, consolidating the servers 106 in this way may improve system manageability, data security, the physical security of the system, and system performance by locating servers 106 and high performance storage systems on localized high performance networks. Centralizing the servers 106 and storage systems and coupling them with advanced system management tools allows more efficient use of server resources.

The servers 106 of each machine farm 38 do not need to be physically proximate to another server 106 in the same machine farm 38. Thus, the group of servers 106 logically grouped as a machine farm 38 may be interconnected using a wide-area network (WAN) connection or a metropolitan-area network (MAN) connection. For example, a machine farm 38 may include servers 106 physically located in different continents or different regions of a continent, country, state, city, campus, or room. Data transmission speeds between servers 106 in the machine farm 38 can be increased if the servers 106 are connected using a local-area network (LAN) connection or some form of direct connection. Additionally, a heterogeneous machine farm 38 may include one or more servers 106 operating according to a type of operating system, while one or more other servers 106 execute one or more types of hypervisors rather than operating systems. In these embodiments, hypervisors may be used to emulate virtual hardware, partition physical hardware, virtualize physical hardware, and execute virtual machines that provide access to computing environments, allowing multiple operating systems to run concurrently on a host computer. Native hypervisors may run directly on the host computer. Hypervisors may include VMware ESX/ESXi, manufactured by VMWare, Inc., of Palo Alto, California; the Xen hypervisor, an open source product whose development is overseen by Citrix Systems, Inc.; the HYPER-V hypervisors provided by Microsoft or others. Hosted hypervisors may run within an operating system on a second software level. Examples of hosted hypervisors may include VMware Workstation and VIRTUALBOX.

Management of the machine farm 38 may be de-centralized. For example, one or more servers 106 may comprise components, subsystems and modules to support one or more management services for the machine farm 38. In one of these embodiments, one or more servers 106 provide functionality for management of dynamic data, including techniques for handling failover, data replication, and increasing the robustness of the machine farm 38. Each server 106 may communicate with a persistent store and, in some embodiments, with a dynamic store.

Server 106 may be a file server, application server, web server, proxy server, appliance, network appliance, gateway, gateway server, virtualization server, deployment server, SSL VPN server, or firewall. In one embodiment, the server 106 may be referred to as a remote machine or a node. In another embodiment, a plurality of nodes 290 may be in the path between any two communicating servers.

Referring to FIG. 23B, a cloud computing environment is depicted. A cloud computing environment may provide client 102 with one or more resources provided by a network environment. The cloud computing environment may include one or more clients 102a-102n, in communication with the cloud 108 over one or more networks 104. Clients 102 may include, e.g., thick clients, thin clients, and zero clients. A thick client may provide at least some functionality even when disconnected from the cloud 108 or servers 106. A thin client or a zero client may depend on the connection to the cloud 108 or server 106 to provide functionality. A zero client may depend on the cloud 108 or other networks 104 or servers 106 to retrieve operating system data for the client device. The cloud 108 may include back end platforms, e.g., servers 106, storage, server farms or data centers.

The cloud 108 may be public, private, or hybrid. Public clouds may include public servers 106 that are maintained by third parties to the clients 102 or the owners of the clients. The servers 106 may be located off-site in remote geographical locations as disclosed above or otherwise. Public clouds may be connected to the servers 106 over a public network. Private clouds may include private servers 106 that are physically maintained by clients 102 or owners of clients. Private clouds may be connected to the servers 106 over a private network 104. Hybrid clouds 108 may include both the private and public networks 104 and servers 106.

The cloud 108 may also include a cloud based delivery, e.g. Software as a Service (SaaS) 110, Platform as a Service (PaaS) 112, and Infrastructure as a Service (IaaS) 114. IaaS may refer to a user renting the use of infrastructure resources that are needed during a specified time period. IaaS providers may offer storage, networking, servers or virtualization resources from large pools, allowing the users to quickly scale up by accessing more resources as needed. Examples of IaaS can include infrastructure and services (e.g., EG-32) provided by OVH HOSTING of Montreal, Quebec, Canada, AMAZON WEB SERVICES provided by Amazon.com, Inc., of Seattle, Washington, RACKSPACE CLOUD provided by Rackspace US, Inc., of San Antonio, Texas, Google Compute Engine provided by Google Inc. of Mountain View, California, or RIGHTSCALE provided by RightScale, Inc., of Santa Barbara, California. PaaS providers may offer functionality provided by IaaS, including, e.g., storage, networking, servers or virtualization, as well as additional resources such as, e.g., the operating system, middleware, or runtime resources. Examples of PaaS include WINDOWS AZURE provided by Microsoft Corporation of Redmond, Washington, Google App Engine provided by Google Inc., and HEROKU provided by Heroku, Inc. of San Francisco, California. SaaS providers may offer the resources that PaaS provides, including storage, networking, servers, virtualization, operating system, middleware, or runtime resources. In some embodiments, SaaS providers may offer additional resources including, e.g., data and application resources. Examples of SaaS include GOOGLE APPS provided by Google Inc., SALESFORCE provided by Salesforce.com Inc. of San Francisco, California, or OFFICE 365 provided by Microsoft Corporation. Examples of SaaS may also include data storage providers, e.g. DROPBOX provided by Dropbox, Inc. of San Francisco, California, Microsoft SKYDRIVE provided by Microsoft Corporation, Google Drive provided by Google Inc., or Apple ICLOUD provided by Apple Inc. of Cupertino, California.

Clients 102 may access IaaS resources with one or more IaaS standards, including, e.g., Amazon Elastic Compute Cloud (EC2), Open Cloud Computing Interface (OCCI), Cloud Infrastructure Management Interface (CIMI), or OpenStack standards. Some IaaS standards may allow clients access to resources over HTTP, and may use Representational State Transfer (REST) protocol or Simple Object Access Protocol (SOAP). Clients 102 may access PaaS resources with different PaaS interfaces. Some PaaS interfaces use HTTP packages, standard Java APIs, JavaMail API, Java Data Objects (JDO), Java Persistence API (JPA), Python APIs, web integration APIs for different programming languages including, e.g., Rack for Ruby, WSGI for Python, or PSGI for Perl, or other APIs that may be built on REST, HTTP, XML, or other protocols. Clients 102 may access SaaS resources through the use of web-based user interfaces, provided by a web browser (e.g. GOOGLE CHROME, Microsoft INTERNET EXPLORER, or Mozilla Firefox provided by Mozilla Foundation of Mountain View, California). Clients 102 may also access SaaS resources through smartphone or tablet applications, including, e.g., Salesforce Sales Cloud, or Google Drive app. Clients 102 may also access SaaS resources through the client operating system, including, e.g., Windows file system for DROPBOX.

In some embodiments, access to IaaS, PaaS, or SaaS resources may be authenticated. For example, a server or authentication server may authenticate a user via security certificates, HTTPS, or API keys. API keys may include various encryption standards such as, e.g., Advanced Encryption Standard (AES). Data resources may be sent over Transport Layer Security (TLS) or Secure Sockets Layer (SSL).

The client 102 and server 106 may be deployed as and/or executed on any type and form of computing device, e.g. a computer, network device or appliance capable of communicating on any type and form of network and performing the operations described herein. FIGS. 23C and 23D depict block diagrams of a computing device 100 useful for practicing an embodiment of the client 102 or a server 106. As shown in FIGS. 23C and 23D, each computing device 100 includes a central processing unit 121, and a main memory unit 122. As shown in FIG. 23C, a computing device 100 may include a storage device 128, an installation device 116, a network interface 118, an I/O controller 123, display devices 124a-124n, a keyboard 126 and a pointing device 127, e.g. a mouse. The storage device 128 may include, without limitation, an operating system, software, and a software of a genomic data processing system 120. As shown in FIG. 23D, each computing device 100 may also include additional optional elements, e.g. a memory port 103, a bridge 170, one or more input/output devices 130a-130n (generally referred to using reference numeral 130), and a cache memory 140 in communication with the central processing unit 121.

The central processing unit 121 is any logic circuitry that responds to and processes instructions fetched from the main memory unit 122. In many embodiments, the central processing unit 121 is provided by a microprocessor unit, e.g.: those manufactured by Intel Corporation of Mountain View, California; those manufactured by Motorola Corporation of Schaumburg, Illinois; the ARM processor and TEGRA system on a chip (SoC) manufactured by Nvidia of Santa Clara, California; the POWER7 processor, those manufactured by International Business Machines of White Plains, New York; or those manufactured by Advanced Micro Devices of Sunnyvale, California. The computing device 100 may be based on any of these processors, or any other processor capable of operating as described herein. The central processing unit 121 may utilize instruction level parallelism, thread level parallelism, different levels of cache, and multi-core processors. A multi-core processor may include two or more processing units on a single computing component. Examples of multi-core processors include the AMD PHENOM IIX2, INTEL CORE i5 and INTEL CORE i7.

Main memory unit or memory device 122 may include one or more memory chips capable of storing data and allowing any storage location to be directly accessed by the microprocessor 121. Main memory unit or device 122 may be volatile and faster than storage 128 memory. Main memory units or devices 122 may be Dynamic random access memory (DRAM) or any variants, including static random access memory (SRAM), Burst SRAM or SynchBurst SRAM (BSRAM), Fast Page Mode DRAM (FPM DRAM), Enhanced DRAM (EDRAM), Extended Data Output RAM (EDO RAM), Extended Data Output DRAM (EDO DRAM), Burst Extended Data Output DRAM (BEDO DRAM), Single Data Rate Synchronous DRAM (SDR SDRAM), Double Data Rate SDRAM (DDR SDRAM), Direct Rambus DRAM (DRDRAM), or Extreme Data Rate DRAM (XDR DRAM). In some embodiments, the main memory 122 or the storage 128 may be non-volatile; e.g., non-volatile read access memory (NVRAM), flash memory non-volatile static RAM (nvSRAM), Ferroelectric RAM (FeRAM), Magnetoresistive RAM (MRAM), Phase-change memory (PRAM), conductive-bridging RAM (CBRAM), Silicon-Oxide-Nitride-Oxide-Silicon (SONOS), Resistive RAM (RRAM), Racetrack, Nano-RAM (NRAM), or Millipede memory. The main memory 122 may be based on any of the above described memory chips, or any other available memory chips capable of operating as described herein. In the embodiment shown in FIG. 23C, the processor 121 communicates with main memory 122 via a system bus 150 (described in more detail below). FIG. 23D depicts an embodiment of a computing device 100 in which the processor communicates directly with main memory 122 via a memory port 103. For example, in FIG. 23D the main memory 122 may be DRDRAM.

FIG. 23D depicts an embodiment in which the main processor 121 communicates directly with cache memory 140 via a secondary bus, sometimes referred to as a backside bus. In other embodiments, the main processor 121 communicates with cache memory 140 using the system bus 150. Cache memory 140 typically has a faster response time than main memory 122 and is typically provided by SRAM, BSRAM, or EDRAM. In the embodiment shown in FIG. 23D, the processor 121 communicates with various I/O devices 130 via a local system bus 150. Various buses may be used to connect the central processing unit 121 to any of the I/O devices 130, including a PCI bus, a PCI-X bus, or a PCI-Express bus, or a NuBus. For embodiments in which the I/O device is a video display 124, the processor 121 may use an Advanced Graphics Port (AGP) to communicate with the display 124 or the I/O controller 123 for the display 124. FIG. 23D depicts an embodiment of a computer 100 in which the main processor 121 communicates directly with I/O device 130b or other processors 121′ via HYPERTRANSPORT, RAPIDIO, or INFINIBAND communications technology. FIG. 23D also depicts an embodiment in which local busses and direct communication are mixed: the processor 121 communicates with I/O device 130a using a local interconnect bus while communicating with I/O device 130b directly.

A wide variety of I/O devices 130a-130n may be present in the computing device 100. Input devices may include keyboards, mice, trackpads, trackballs, touchpads, touch mice, multi-touch touchpads and touch mice, microphones, multi-array microphones, drawing tablets, cameras, single-lens reflex camera (SLR), digital SLR (DSLR), CMOS sensors, accelerometers, infrared optical sensors, pressure sensors, magnetometer sensors, angular rate sensors, depth sensors, proximity sensors, ambient light sensors, gyroscopic sensors, or other sensors. Output devices may include video displays, graphical displays, speakers, headphones, inkjet printers, laser printers, and 3D printers.

Devices 130a-130n may include a combination of multiple input or output devices, including, e.g., Microsoft KINECT, Nintendo Wiimote for the WII, Nintendo WII U GAMEPAD, or Apple IPHONE. Some devices 130a-130n allow gesture recognition inputs through combining some of the inputs and outputs. Some devices 130a-130n provides for facial recognition which may be utilized as an input for different purposes including authentication and other commands. Some devices 130a-130n provides for voice recognition and inputs, including, e.g., Microsoft KINECT, SIRI for IPHONE by Apple, Google Now or Google Voice Search.

Additional devices 130a-130n have both input and output capabilities, including, e.g., haptic feedback devices, touchscreen displays, or multi-touch displays. Touchscreen, multi-touch displays, touchpads, touch mice, or other touch sensing devices may use different technologies to sense touch, including, e.g., capacitive, surface capacitive, projected capacitive touch (PCT), in-cell capacitive, resistive, infrared, waveguide, dispersive signal touch (DST), in-cell optical, surface acoustic wave (SAW), bending wave touch (BWT), or force-based sensing technologies. Some multi-touch devices may allow two or more contact points with the surface, allowing advanced functionality including, e.g., pinch, spread, rotate, scroll, or other gestures. Some touchscreen devices, including, e.g., Microsoft PIXELSENSE or Multi-Touch Collaboration Wall, may have larger surfaces, such as on a table-top or on a wall, and may also interact with other electronic devices. Some I/O devices 130a-130n, display devices 124a-124n or group of devices may be augment reality devices. The I/O devices may be controlled by an I/O controller 123 as shown in FIG. 23C. The I/O controller may control one or more I/O devices, such as, e.g., a keyboard 126 and a pointing device 127, e.g., a mouse or optical pen. Furthermore, an I/O device may also provide storage and/or an installation medium 116 for the computing device 100. In still other embodiments, the computing device 100 may provide USB connections (not shown) to receive handheld USB storage devices. In further embodiments, an I/O device 130 may be a bridge between the system bus 150 and an external communication bus, e.g. a USB bus, a SCSI bus, a FireWire bus, an Ethernet bus, a Gigabit Ethernet bus, a Fibre Channel bus, or a Thunderbolt bus.

In some embodiments, display devices 124a-124n may be connected to I/O controller 123. Display devices may include, e.g., liquid crystal displays (LCD), thin film transistor LCD (TFT-LCD), blue phase LCD, electronic papers (e-ink) displays, flexile displays, light emitting diode displays (LED), digital light processing (DLP) displays, liquid crystal on silicon (LCOS) displays, organic light-emitting diode (OLED) displays, active-matrix organic light-emitting diode (AMOLED) displays, liquid crystal laser displays, time-multiplexed optical shutter (TMOS) displays, or 3D displays. Examples of 3D displays may use, e.g. stereoscopy, polarization filters, active shutters, or autostereoscopy. Display devices 124a-124n may also be a head-mounted display (HMD). In some embodiments, display devices 124a-124n or the corresponding I/O controllers 123 may be controlled through or have hardware support for OPENGL or DTRECTX API or other graphics libraries.

In some embodiments, the computing device 100 may include or connect to multiple display devices 124a-124n, which each may be of the same or different type and/or form. As such, any of the I/O devices 130a-130n and/or the I/O controller 123 may include any type and/or form of suitable hardware, software, or combination of hardware and software to support, enable or provide for the connection and use of multiple display devices 124a-124n by the computing device 100. For example, the computing device 100 may include any type and/or form of video adapter, video card, driver, and/or library to interface, communicate, connect or otherwise use the display devices 124a-124n. In one embodiment, a video adapter may include multiple connectors to interface to multiple display devices 124a-124n. In other embodiments, the computing device 100 may include multiple video adapters, with each video adapter connected to one or more of the display devices 124a-124n. In some embodiments, any portion of the operating system of the computing device 100 may be configured for using multiple displays 124a-124n. In other embodiments, one or more of the display devices 124a-124n may be provided by one or more other computing devices 100a or 100b connected to the computing device 100, via the network 104. In some embodiments software may be designed and constructed to use another computer's display device as a second display device 124a for the computing device 100. For example, in one embodiment, an Apple iPad may connect to a computing device 100 and use the display of the device 100 as an additional display screen that may be used as an extended desktop. One ordinarily skilled in the art will recognize and appreciate the various ways and embodiments that a computing device 100 may be configured to have multiple display devices 124a-124n.

Referring again to FIG. 23C, the computing device 100 may comprise a storage device 128 (e.g. one or more hard disk drives or redundant arrays of independent disks) for storing an operating system or other related software, and for storing application software programs such as any program related to the software for the genomic data processing system 120. Examples of storage device 128 include, e.g., hard disk drive (HDD); optical drive including CD drive, DVD drive, or BLU-RAY drive; solid-state drive (SSD); USB flash drive; or any other device suitable for storing data. Some storage devices may include multiple volatile and non-volatile memories, including, e.g., solid state hybrid drives that combine hard disks with solid state cache. Some storage device 128 may be non-volatile, mutable, or read-only. Some storage device 128 may be internal and connect to the computing device 100 via a bus 150. Some storage devices 128 may be external and connect to the computing device 100 via an I/O device 130 that provides an external bus. Some storage device 128 may connect to the computing device 100 via the network interface 118 over a network 104, including, e.g., the Remote Disk for MACBOOK AIR by Apple. Some client devices 100 may not require a non-volatile storage device 128 and may be thin clients or zero clients 102. Some storage device 128 may also be used as an installation device 116, and may be suitable for installing software and programs. Additionally, the operating system and the software can be run from a bootable medium, for example, a bootable CD, e.g. KNOPPIX, a bootable CD for GNU/Linux that is available as a GNU/Linux distribution from knoppix.net.

Client device 100 may also install software or application from an application distribution platform. Examples of application distribution platforms include the App Store for iOS provided by Apple, Inc., the Mac App Store provided by Apple, Inc., GOOGLE PLAY for Android OS provided by Google Inc., Chrome Webstore for CHROME OS provided by Google Inc., and Amazon Appstore for Android OS and KINDLE FIRE provided by Amazon.com, Inc. An application distribution platform may facilitate installation of software on a client device 102. An application distribution platform may include a repository of applications on a server 106 or a cloud 108, which the clients 102a-102n may access over a network 104. An application distribution platform may include application developed and provided by various developers. A user of a client device 102 may select, purchase and/or download an application via the application distribution platform.

Furthermore, the computing device 100 may include a network interface 118 to interface to the network 104 through a variety of connections including, but not limited to, standard telephone lines LAN or WAN links (e.g., 802.11, T1, T3, Gigabit Ethernet, Infiniband), broadband connections (e.g., ISDN, Frame Relay, ATM, Gigabit Ethernet, Ethernet-over-SONET, ADSL, VDSL, BPON, GPON, fiber optical including FiOS), wireless connections, or some combination of any or all of the above. Connections can be established using a variety of communication protocols (e.g., TCP/IP, Ethernet, ARCNET, SONET, SDH, Fiber Distributed Data Interface (FDDI), IEEE 802.11a/b/g/n/ac CDMA, GSM, WiMax and direct asynchronous connections). In one embodiment, the computing device 100 communicates with other computing devices 100′ via any type and/or form of gateway or tunneling protocol e.g. Secure Socket Layer (SSL) or Transport Layer Security (TLS), or the Citrix Gateway Protocol manufactured by Citrix Systems, Inc. of Ft. Lauderdale, Florida. The network interface 118 may comprise a built-in network adapter, network interface card, PCMCIA network card, EXPRESSCARD network card, card bus network adapter, wireless network adapter, USB network adapter, modem or any other device suitable for interfacing the computing device 100 to any type of network capable of communication and performing the operations described herein.

A computing device 100 of the sort depicted in FIGS. 23B and 23C may operate under the control of an operating system, which controls scheduling of tasks and access to system resources. The computing device 100 can be running any operating system such as any of the versions of the MICROSOFT WINDOWS operating systems, the different releases of the Unix and Linux operating systems, any version of the MAC OS for Macintosh computers, any embedded operating system, any real-time operating system, any open source operating system, any proprietary operating system, any operating systems for mobile computing devices, or any other operating system capable of running on the computing device and performing the operations described herein. Typical operating systems include, but are not limited to: WINDOWS 2000, WINDOWS Server 2022, WINDOWS CE, WINDOWS Phone, WINDOWS XP, WINDOWS VISTA, and WINDOWS 7, WINDOWS RT, WINDOWS 8, and WINDOWS 10, all of which are manufactured by Microsoft Corporation of Redmond, Washington; MAC OS and iOS, manufactured by Apple, Inc. of Cupertino, California; and Linux, a freely-available operating system, e.g. Linux Mint distribution (“distro”) or Ubuntu, distributed by Canonical Ltd. of London, United Kingdom; or Unix or other Unix-like derivative operating systems; and Android, designed by Google, of Mountain View, California, among others. Some operating systems, including, e.g., the CHROME OS by Google, may be used on zero clients or thin clients, including, e.g., CHROMEBOOKS.

The computer system 100 can be any workstation, telephone, desktop computer, laptop or notebook computer, netbook, ULTRABOOK, tablet, server, handheld computer, mobile telephone, smartphone or other portable telecommunications device, media playing device, a gaming system, mobile computing device, or any other type and/or form of computing, telecommunications or media device that is capable of communication. The computer system 100 has sufficient processor power and memory capacity to perform the operations described herein. The computer system 100 can be of any suitable size, such as a standard desktop computer or a Raspberry Pi 4 manufactured by Raspberry Pi Foundation, of Cambridge, United Kingdom. In some embodiments, the computing device 100 may have different processors, operating systems, and input devices consistent with the device. The Samsung GALAXY smartphones, e.g., operate under the control of Android operating system developed by Google, Inc. GALAXY smartphones receive input via a touch interface.

In some embodiments, the computing device 100 is a gaming system. For example, the computer system 100 may comprise a PLAYSTATION 3, or PERSONAL PLAYSTATION PORTABLE (PSP), or a PLAYSTATION VITA device manufactured by the Sony Corporation of Tokyo, Japan, a NINTENDO DS, NINTENDO 3DS, NINTENDO WII, or a NINTENDO WII U device manufactured by Nintendo Co., Ltd., of Kyoto, Japan, an XBOX 360 device manufactured by the Microsoft Corporation of Redmond, Washington.

In some embodiments, the computing device 100 is a digital audio player such as the Apple IPOD, IPOD Touch, and IPOD NANO lines of devices, manufactured by Apple Computer of Cupertino, California. Some digital audio players may have other functionality, including, e.g., a gaming system or any functionality made available by an application from a digital application distribution platform. For example, the IPOD Touch may access the Apple App Store. In some embodiments, the computing device 100 is a portable media player or digital audio player supporting file formats including, but not limited to, MP3, WAV, M4A/AAC, WMA Protected AAC, AIFF, Audible audiobook, Apple Lossless audio file formats and .mov, .m4v, and .mp4 MPEG-4 (H.264/MPEG-4 AVC) video file formats.

In some embodiments, the computing device 100 is a tablet e.g. the IPAD line of devices by Apple; GALAXY TAB family of devices by Samsung; or KINDLE FIRE, by Amazon.com, Inc. of Seattle, Washington. In other embodiments, the computing device 100 is an eBook reader, e.g. the KINDLE family of devices by Amazon.com, or NOOK family of devices by Barnes & Noble, Inc. of New York City, New York.

In some embodiments, the communications device 102 includes a combination of devices, e.g. a smartphone combined with a digital audio player or portable media player. For example, one of these embodiments is a smartphone, e.g. the IPHONE family of smartphones manufactured by Apple, Inc.; a Samsung GALAXY family of smartphones manufactured by Samsung, Inc.; or a Motorola DROID family of smartphones. In yet another embodiment, the communications device 102 is a laptop or desktop computer equipped with a web browser and a microphone and speaker system, e.g. a telephony headset. In these embodiments, the communications devices 102 are web-enabled and can receive and initiate phone calls. In some embodiments, a laptop or desktop computer is also equipped with a webcam or other video capture device that enables video chat and video call.

In some embodiments, the status of one or more machines 102, 106 in the network 104 are monitored, generally as part of network management. In one of these embodiments, the status of a machine may include an identification of load information (e.g., the number of processes on the machine, CPU and memory utilization), of port information (e.g., the number of available communication ports and the port addresses), or of session status (e.g., the duration and type of processes, and whether a process is active or idle). In another of these embodiments, this information may be identified by a plurality of metrics, and the plurality of metrics can be applied at least in part towards decisions in load distribution, network traffic management, and network failure recovery as well as any aspects of operations of the present solution described herein. Aspects of the operating environments and components described above will become apparent in the context of the systems and methods disclosed herein.

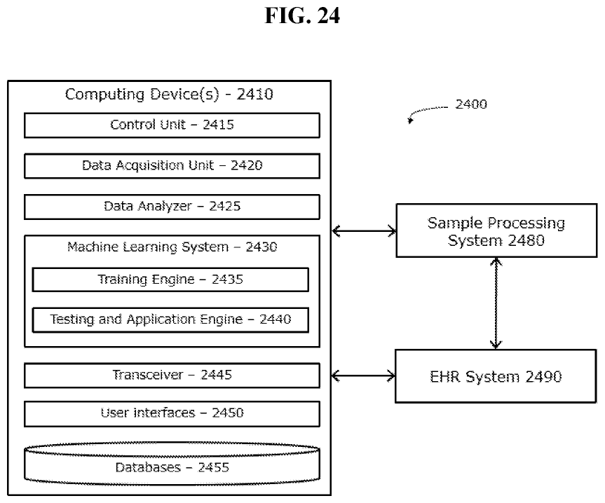

Referring to FIG. 24, in various embodiments, a system 2400 may include a computing device 2410 (or multiple computing devices, co-located or remote to each other), a sample processing system 2480, and an electronic health record (EHR) system 2490. In various embodiments, computing device 2410 (or components thereof) may be integrated with the sample processing system 2480 (or components thereof) and/or EHR system 2490 (or components thereof). In various embodiments, the sample processing system 2480 may include, may be, or may employ, in situ hybridization, PCR, Next-generation sequencing, Northern blotting, microarray, dot or slot blots, FISH, electrophoresis, chromatography, and/or mass spectroscopy on such biological sample as blood, plasma, serum, and/or tissue. For example, in certain embodiments, the sample processing system 2490 may be or may include a Next-generation sequencer. In various embodiments, the EHR system 2490 may include, may be, or may employ, various computing devices that include health records of patients and study subjects (including devices of hospitals, clinics, healthcare practitioners, etc.), obtained from various sources, such as entries by healthcare practitioners, sample processing system 2480, university and hospital systems, government agency systems, etc.

In various embodiments, the computing device 2410 (or multiple computing devices) may be used to control, and receive signals acquired via, components of sample processing system 2480. The computing device 2410 may include one or more processors and one or more volatile and non-volatile memories for storing computing code and data that are captured, acquired, recorded, and/or generated. The computing device 2410 may include a control unit 2415 that in certain embodiments may be configured to exchange control signals with sample processing system 2480, allowing the computing device 2410 to be used to control, for example, processing of samples and/or delivery of data generated and/or acquired through processing of samples.

In various embodiments, computing device 2410 may include a data acquisition unit 2420 that may be configured to exchange control signals, or otherwise communicate, with sample processing system 2480 (or components thereof) and/or EHR system 2490, allowing the computing device 2410 to be used to control the capture of physiological data and/or signals via sensors of the sample processing system 2480, retrieve data or signals (e.g., from sample processing system 2480, EHR system 2490, and/or memory devices where data is stored), and direct transfer of data or signals (e.g., to sample processing system 2490 as feedback thereto, to EHR system 2490, to memory for storage, and/or to other systems or devices).

In various embodiment, a data analyzer 2425 may direct analysis of the data and signals, and output analysis results. Data analyzer 2425 may be used, for example, to transform raw data captured or obtained via sample processing system 2480 and/or EHR system 2490, and may employ pre-processing procedures involved in generating a training dataset. For example, in some implementations, data may be generated as a multi-dimensional array or vector with values representing, and to prevent the machine learning system from overemphasizing certain readings, values may be normalized to a predetermined range (e.g. 0-1, 0-100, or any other such range). The normalization may comprise linear rescaling, or may be a more complex function. In some implementations, dimension reduction may be performed to reduce large and sparse arrays or vectors. In some implementations, feature recognition may be performed to select a subset of features for further analysis, such as principal component analysis.

In various embodiments, a machine learning system 2430 may be used to implement various machine learning functionality discussed herein. Machine learning system 2430 may include a training engine 2435 configured to train predictive models using, for example, data obtained from or via data acquisition unit 2420 and/or processed data obtained from or via data analyzer 2425. The training engine 2435 may, for example, generate or obtain training datasets from or via data analyzer 2425 and may perform validation of datasets. The training engine 2435 may comprise a feature analyzer used to evaluate features by, for example, quantifying the impact of each feature on the developed model. Such a feature analyzer may, for example, uncover clinically important features that were globally predictive of the outcome, and may determine, for example, contributions of all features, or the top features (e.g., the top 2, top 5, top 10, top 15, top 20, top 25, top 30, etc.) on individual predictions. Features may be selected based on a threshold, such a percent contribution to predicting a medical condition, such as 0.5%, 1%, 2%, 5%, 10%, etc. A testing and application engine 2440 may be configured to test and apply models trained via training engine 2435 to, for example, study subject and/or patient data from data acquisition unit 2420 and/or data analyzer 2425.

In various embodiments, a transceiver 2445 allows the computing device 2410 to exchange readings, control commands, and/or other data with sample processing system 2480 (or components thereof) and/or EHR system 2490 (or components thereof). The transceiver 2445 may additionally or alternatively include a network interface permitting the computing device 2410 to communicate with other remote devices and systems via, for example, a telecommunications network such as the internet. One or more user interfaces 2450 allow the computing device 2410 to receive user inputs (e.g., via a keyboard, touchscreen, microphone, camera, etc.) and provide outputs (e.g., via a touchscreen or other display screen, audio speakers, haptic devices, etc.). A display screen may be employed, for example, to provide real time or near real time waveforms or other readings or measurements obtained via sensors being used to capture physiological data from subjects and patients. The computing device 2410 may additionally include one or more databases 2455 (stored in, e.g., one or more computer-readable non-volatile memory devices) for storing, for example, data and analyses obtained from or via data acquisition unit 2420, data analyzer 2425, machine learning system 2430 (e.g., training engine 2435 and/or testing and application engine 2440), sample processing system 2480, and/or EHR system 2490. In some implementations, database 2455 (or portions thereof) may alternatively or additionally be part of another computing device that is co-located or remote and in communication with computing device 2410, sample processing system 2480 (or components thereof), and/or EHR system 2490.

In one aspect, the present disclosure provides a method of training a machine learning classifier for predicting responsiveness of cancer patients to an immune checkpoint blockade (ICB) therapy, the method comprising: receiving data on a cohort of subjects, the subjects in the cohort having a plurality of cancer types; generating a training dataset based on the received data, the training dataset comprising a plurality of features for each subject in the cohort, the plurality of features comprising at least one of a blood albumin level, a blood hemoglobin level, or a blood platelet level; and applying a machine learning method to the training dataset to develop the machine learning classifier for predicting responsiveness of cancer patients to the ICB therapy, wherein applying the machine learning method comprises: applying a machine learning technique to the training dataset to each cancer type individually; performing hyperparameter optimization to identify one or more machine learning models with an accuracy that exceeds an accuracy threshold for the classifier; and determining an optimal operating-point threshold based on optimization of sensitivity and specificity of the receiver operating characteristic (ROC) curves for the training dataset; and wherein the classifier is configured to receive the plurality of features for cancer patients and generate predictors for responsiveness of the cancer patients to the ICB therapy. In some embodiments, the machine learning technique is a random forest technique, and the one or more machine learning models are random forest models. The ICB therapy may be a PD-1/PD-L1 inhibitor, a CTLA-4 inhibitor, or a combination thereof. In some embodiments, the ICB therapy comprises one or more of pembrolizumab, nivolumab, cemiplimab, atezolizumab, avelumab, durvalumab, ipilimumab, tremelimumab, ticlimumab, JTX-4014, Spartalizumab (PDR001), Camrelizumab (SHR1210), Sintilimab (IBI308), Tislelizumab (BGB-A317), Toripalimab (JS 001), Dostarlimab (TSR-042, WBP-285), INCMGA00012 (MGA012), AMP-224, AMP-514, KN035, CK-301, AUNP12, CA-170, or BMS-986189. Additionally or alternatively, in some embodiments, the predictors comprise response probability values.

In any and all embodiments of the methods disclosed herein, the plurality of features for each subject in the cohort are determined by assaying blood and/or sequencing tumor DNA.

Additionally or alternatively, in some embodiments, the machine learning classifier is an ensemble learning random forest classifier. In certain embodiments, the machine learning classifier is cancer-type specific, and the plurality of features comprises a cancer type.

Additionally or alternatively, in some embodiments, hyperparameter optimization is performed for each cancer type in the plurality of cancer types. In certain embodiments, performing the hyperparameter optimization comprises performing an exhaustive grid search technique.

In any and all embodiments of the methods disclosed herein, the plurality of features comprises a plurality of at least one genomic feature, at least one molecular feature, at least one clinical feature, and/or at least one demographic feature. In certain embodiments, the plurality of features comprises at least one genomic feature, at least one molecular feature, at least one clinical feature, and at least one demographic feature. In some embodiments, the plurality of features further comprises a plurality of: (i) a tumor mutation burden (TMB) metric, (ii) a fraction of copy-number alteration (FCNA) metric, (iii) an HLA-I evolutionary divergence (HED) metric, (iv) a loss of heterozygosity (LOH) status in HLA-I, (v) a microsatellite instability (MSI) status, (vi) a body mass index (BMI), (vii) a gender, (viii) a blood neutrophil-to-lymphocyte ratio (NLR) metric, (ix) a tumor stage, (x) an immune checkpoint inhibitor, (xi) an age, (xii) a cancer type, and/or (xiii) an indication of whether chemotherapy has been administered. In other embodiments, the plurality of features comprises: (i) a tumor mutation burden (TMB) metric, (ii) a fraction of copy-number alteration (FCNA) metric, (iii) an HLA-I evolutionary divergence (HED) metric, (iv) a loss of heterozygosity (LOH) status in HLA-I, (v) a microsatellite instability (MSI) status, (vi) a body mass index (BMI), (vii) a gender, (viii) a blood neutrophil-to-lymphocyte ratio (NLR) metric, (ix) a tumor stage, (x) an immune checkpoint inhibitor, (xi) an age, (xii) a cancer type, (xiii) an indication of whether chemotherapy has been administered, (xiv) the blood albumin level, (xv) the blood hemoglobin level, and (xvi) the blood platelet level. The tumor stage may stage I, stage II, Stage III or stage IV.

Additionally or alternatively, in some embodiments, the plurality of features comprises one or more features having discrete values and/or one or more features having continuous values. In some embodiments, the one or more features having discrete values are selected from among an indication of whether chemotherapy has been administered, cancer type, LOH in HLA-I, an immune checkpoint inhibitor, MSI status, and tumor stage. In certain embodiments, the one or more features having continuous values are selected from among a TMB metric, blood albumin level, blood NLR metric, age, blood hemoglobin level, blood platelet level, a FCNA metric, BMI, and a HED metric.

In any of the preceding embodiments, the method further comprises applying the classifier to data on a cancer patient to generate a predictor, and determining whether the cancer patient is predicted to be responsive to the ICB therapy based on the predictor and the operating-point threshold. In some embodiments, the predictor comprises a response probability value.

Additionally or alternatively, in some embodiments, the method further comprises administering an effective amount of the ICB therapy to the cancer patient predicted to be responsive to the ICB therapy based on the predictor and the operating-point threshold. In any and all embodiments of the methods disclosed herein, the cancer patient predicted to be responsive to the ICB therapy based on the predictor and the operating-point threshold exhibits extended overall survival and/or progression-free survival compared to a cancer patient that is predicted to be non-responsive to the ICB therapy based on the predictor and the operating-point threshold.

In any and all embodiments of the methods disclosed herein, the plurality of cancer types are selected from the group consisting of non-small cell lung cancers (NSCLC), small cell lung cancers (SCLC), melanoma, renal cell carcinoma, bladder cancer, head and neck cancer, sarcoma, endometrial cancer, gastric cancer, hepatobiliary cancer, colorectal cancer, esophageal cancer, pancreatic cancer, mesothelioma, ovarian cancer, and breast cancer.

In one aspect, the present disclosure provides a method of predicting responsiveness of a cancer patient to an immune checkpoint blockade (ICB) therapy using a machine learning classifier, the method comprising: receiving patient data corresponding to a plurality of features for the cancer patient; applying the machine learning classifier to the patient data to generate a predictor; and determining whether the cancer patient is predicted to be responsive to the ICB therapy based on the predictor and an operating-point threshold, wherein the machine learning classifier is trained by: receiving cohort data on a cohort of subjects, the subjects in the cohort having a plurality of cancer types; generating a training dataset based on the received cohort data, the training dataset comprising the plurality of features for each subject in the cohort, the plurality of features comprising at least one of a blood albumin level, a blood hemoglobin level, or a blood platelet level; and applying a machine learning method to the training dataset to develop the machine learning classifier for predicting responsiveness of cancer patients to the ICB therapy, wherein applying the machine learning method comprises: applying a machine learning technique to the training dataset to each cancer type individually; performing hyperparameter optimization to identify one or more machine learning models with an accuracy that exceeds an accuracy threshold for the machine learning classifier; and determining the optimal operating-point threshold based on optimization of sensitivity and specificity of the receiver operating characteristic (ROC) curves for the training dataset; and wherein the machine learning classifier is configured to receive the plurality of features for cancer patients and generate predictors for responsiveness of the cancer patients to the ICB therapy. The ICB therapy may be a PD-1/PD-L1 inhibitor, a CTLA-4 inhibitor, or a combination thereof. In some embodiments, the ICB therapy comprises one or more of pembrolizumab, nivolumab, cemiplimab, atezolizumab, avelumab, durvalumab, ipilimumab, tremelimumab, ticlimumab, JTX-4014, Spartalizumab (PDR001), Camrelizumab (SHR1210), Sintilimab (IBI308), Tislelizumab (BGB-A317), Toripalimab (JS 001), Dostarlimab (TSR-042, WBP-285), INCMGA00012 (MGA012), AMP-224, AMP-514, KN035, CK-301, AUNP12, CA-170, or BMS-986189. Additionally or alternatively, in some embodiments, the predictors comprise response probability values. In certain embodiments, the machine learning technique is a random forest technique, and the one or more machine learning models are random forest models.

Additionally or alternatively, in some embodiments, the method further comprises administering an effective amount of the ICB therapy to the cancer patient predicted to be responsive to the ICB therapy based on the predictor and the operating-point threshold. In any and all embodiments of the methods disclosed herein, the cancer patient predicted to be responsive to the ICB therapy based on the predictor and the operating-point threshold exhibits extended overall survival and/or progression-free survival compared to a cancer patient that is predicted to be non-responsive to the ICB therapy based on the predictor and the operating-point threshold. The predictor may comprise a response probability value.

Additionally or alternatively, in some embodiments, the machine learning classifier is an ensemble learning random forest classifier. In certain embodiments, the machine learning classifier is cancer-type specific, and the plurality of features comprises a cancer type.

Additionally or alternatively, in some embodiments, hyperparameter optimization is performed for each cancer type in the plurality of cancer types. In certain embodiments, performing the hyperparameter optimization comprises performing an exhaustive grid search technique.

In any and all embodiments of the methods disclosed herein, the plurality of features comprises a plurality of at least one genomic feature, at least one molecular feature, at least one clinical feature, and/or at least one demographic feature. In certain embodiments, the plurality of features comprises at least one genomic feature, at least one molecular feature, at least one clinical feature, and at least one demographic feature. In some embodiments, the plurality of features further comprises a plurality of: (i) a tumor mutation burden (TMB) metric, (ii) a fraction of copy-number alteration (FCNA) metric, (iii) an HLA-I evolutionary divergence (HED) metric, (iv) a loss of heterozygosity (LOH) status in HLA-I, (v) a microsatellite instability (MSI) status, (vi) a body mass index (BMI), (vii) a gender, (viii) a blood neutrophil-to-lymphocyte ratio (NLR) metric, (ix) a tumor stage, (x) an immune checkpoint inhibitor, (xi) an age, (xii) a cancer type, and/or (xiii) an indication of whether chemotherapy has been administered. In other embodiments, the plurality of features comprises: (i) a tumor mutation burden (TMB) metric, (ii) a fraction of copy-number alteration (FCNA) metric, (iii) an HLA-I evolutionary divergence (HED) metric, (iv) a loss of heterozygosity (LOH) status in HLA-I, (v) a microsatellite instability (MSI) status, (vi) a body mass index (BMI), (vii) a gender, (viii) a blood neutrophil-to-lymphocyte ratio (NLR) metric, (ix) a tumor stage, (x) an immune checkpoint inhibitor, (xi) an age, (xii) a cancer type, (xiii) an indication of whether chemotherapy has been administered, (xiv) the blood albumin level, (xv) the blood hemoglobin level, and (xvi) the blood platelet level. The tumor stage may stage I, stage II, Stage III or stage IV.

Additionally or alternatively, in some embodiments, the plurality of features comprises one or more features having discrete values and/or one or more features having continuous values. In some embodiments, the one or more features having discrete values are selected from among an indication of whether chemotherapy has been administered, cancer type, LOH in HLA-I, an immune checkpoint inhibitor, MSI status, and tumor stage. In certain embodiments, the one or more features having continuous values are selected from among a TMB metric, blood albumin level, blood NLR metric, age, blood hemoglobin level, blood platelet level, a FCNA metric, BMI, and a HED metric.

In any and all embodiments of the methods disclosed herein, the plurality of cancer types are selected from the group consisting of non-small cell lung cancers (NSCLC), small cell lung cancers (SCLC), melanoma, renal cell carcinoma, bladder cancer, head and neck cancer, sarcoma, endometrial cancer, gastric cancer, hepatobiliary cancer, colorectal cancer, esophageal cancer, pancreatic cancer, mesothelioma, ovarian cancer, and breast cancer.

In any and all embodiments of the methods disclosed herein, the plurality of features for each subject in the cohort are determined by assaying blood and/or sequencing tumor DNA. In certain embodiments, the plurality of features for the cancer patient are determined by assaying blood and/or sequencing tumor DNA.

In another aspect, the present disclosure provides a machine learning system for training a machine learning classifier for predicting responsiveness of cancer patients to an immune checkpoint blockade (ICB) therapy, the system comprising a processor and a memory with instructions which, when executed by the processor, cause the processor to: receive data on a cohort of subjects, the subjects in the cohort having a plurality of cancer types; generate a training dataset based on the received data, the training dataset comprising a plurality of features for each subject in the cohort, the plurality of features comprising at least one of a blood albumin level, a blood hemoglobin level, or a blood platelet level; and apply a machine learning method to the training dataset to develop the machine learning classifier for predicting responsiveness of cancer patients to the ICB therapy; wherein applying the machine learning method comprises: applying a machine learning technique to the training dataset to each cancer type individually; performing hyperparameter optimization to identify one or more machine learning models with an accuracy that exceeds an accuracy threshold for the machine learning classifier; and determining an optimal operating-point threshold based on optimization of sensitivity and specificity of the receiver operating characteristic (ROC) curves for the training dataset; and wherein the machine learning classifier is configured to receive the plurality of features for cancer patients and generate predictors for responsiveness of the cancer patients to the ICB therapy. In some embodiments, the machine learning technique is a random forest technique, and the one or more machine learning models are random forest models. The ICB therapy may be a PD-1/PD-L1 inhibitor, a CTLA-4 inhibitor, or a combination thereof. In some embodiments, the ICB therapy comprises one or more of pembrolizumab, nivolumab, cemiplimab, atezolizumab, avelumab, durvalumab, ipilimumab, tremelimumab, ticlimumab, JTX-4014, Spartalizumab (PDR001), Camrelizumab (SHR1210), Sintilimab (IBI308), Tislelizumab (BGB-A317), Toripalimab (JS 001), Dostarlimab (TSR-042, WBP-285), INCMGA00012 (MGA012), AMP-224, AMP-514, KN035, CK-301, AUNP12, CA-170, or BMS-986189.

Additionally or alternatively, in some embodiments, the machine learning classifier is an ensemble learning random forest classifier. In certain embodiments, the machine learning classifier is cancer-type specific, and the plurality of features comprises a cancer type.

Additionally or alternatively, in some embodiments, hyperparameter optimization is performed for each cancer type in the plurality of cancer types. In certain embodiments, performing the hyperparameter optimization comprises performing an exhaustive grid search technique.

In any and all embodiments of the machine learning systems disclosed herein, the plurality of features comprises a plurality of at least one genomic feature, at least one molecular feature, at least one clinical feature, and/or at least one demographic feature. In certain embodiments, the plurality of features comprises at least one genomic feature, at least one molecular feature, at least one clinical feature, and at least one demographic feature. In some embodiments, the plurality of features further comprises a plurality of: (i) a tumor mutation burden (TMB) metric, (ii) a fraction of copy-number alteration (FCNA) metric, (iii) an HLA-I evolutionary divergence (HED) metric, (iv) a loss of heterozygosity (LOH) status in HLA-I, (v) a microsatellite instability (MSI) status, (vi) a body mass index (BMI), (vii) a gender, (viii) a blood neutrophil-to-lymphocyte ratio (NLR) metric, (ix) a tumor stage, (x) an immune checkpoint inhibitor, (xi) an age, (xii) a cancer type, and/or (xiii) an indication of whether chemotherapy has been administered. In other embodiments, the plurality of features comprises: (i) a tumor mutation burden (TMB) metric, (ii) a fraction of copy-number alteration (FCNA) metric, (iii) an HLA-I evolutionary divergence (HED) metric, (iv) a loss of heterozygosity (LOH) status in HLA-I, (v) a microsatellite instability (MSI) status, (vi) a body mass index (BMI), (vii) a gender, (viii) a blood neutrophil-to-lymphocyte ratio (NLR) metric, (ix) a tumor stage, (x) an immune checkpoint inhibitor, (xi) an age, (xii) a cancer type, (xiii) an indication of whether chemotherapy has been administered, (xiv) the blood albumin level, (xv) the blood hemoglobin level, and (xvi) the blood platelet level. The tumor stage may stage I, stage II, Stage III or stage IV.

Additionally or alternatively, in some embodiments, the plurality of features comprises one or more features having discrete values and/or one or more features having continuous values. In some embodiments, the one or more features having discrete values are selected from among an indication of whether chemotherapy has been administered, cancer type, LOH in HLA-I, an immune checkpoint inhibitor, MSI status, and tumor stage. In certain embodiments, the one or more features having continuous values are selected from among a TMB metric, blood albumin level, blood NLR metric, age, blood hemoglobin level, blood platelet level, a FCNA metric, BMI, and a HED metric.

Additionally or alternatively, in some embodiments, the instructions further cause the processor to apply the machine learning classifier to data on a cancer patient to generate a predictor, and determine whether the cancer patient is predicted to be responsive to the ICB therapy based on the predictor and the operating-point threshold. The predictor may comprise a response probability value.

In any and all embodiments of the machine learning systems disclosed herein, the plurality of cancer types are selected from the group consisting of non-small cell lung cancers (NSCLC), small cell lung cancers (SCLC), melanoma, renal cell carcinoma, bladder cancer, head and neck cancer, sarcoma, endometrial cancer, gastric cancer, hepatobiliary cancer, colorectal cancer, esophageal cancer, pancreatic cancer, mesothelioma, ovarian cancer, and breast cancer.

In another aspect, the present disclosure provides a computing system for predicting responsiveness of a cancer patient to an immune checkpoint blockade (ICB) therapy, the computing system comprising a processor and a memory with instructions which, when executed by the processor, cause the processor to: receive patient data corresponding to a plurality of features for the cancer patient; apply a machine learning classifier to the patient data to generate a predictor; and determine whether the cancer patient is predicted to be responsive to the ICB therapy based on the predictor and an operating-point threshold, wherein the classifier is trained by: receiving cohort data on a cohort of subjects, the subjects in the cohort having a plurality of cancer types; generating a training dataset based on the received cohort data, the training dataset comprising the plurality of features for each subject in the cohort, the plurality of features comprising at least one of a blood albumin level, a blood hemoglobin level, or a blood platelet level; and applying a machine learning method to the training dataset to develop the machine learning classifier for predicting responsiveness of cancer patients to the ICB therapy, wherein applying the machine learning method comprises: applying a machine learning technique to the training dataset to each cancer type individually; performing hyperparameter optimization to identify one or more machine learning models with an accuracy that exceeds an accuracy threshold for the machine learning classifier; and determining the optimal operating-point threshold based on optimization of sensitivity and specificity of the receiver operating characteristic (ROC) curves for the training dataset; and wherein the machine learning classifier is configured to receive the plurality of features for cancer patients and generate predictors for responsiveness of the cancer patients to the ICB therapy. The predictor may comprise a response probability value. The ICB therapy may be a PD-1/PD-L1 inhibitor, a CTLA-4 inhibitor, or a combination thereof. In some embodiments, the ICB therapy comprises one or more of pembrolizumab, nivolumab, cemiplimab, atezolizumab, avelumab, durvalumab, ipilimumab, tremelimumab, ticlimumab, JTX-4014, Spartalizumab (PDR001), Camrelizumab (SHR1210), Sintilimab (IBI308), Tislelizumab (BGB-A317), Toripalimab (JS 001), Dostarlimab (TSR-042, WBP-285), INCMGA00012 (MGA012), AMP-224, AMP-514, KN035, CK-301, AUNP12, CA-170, or BMS-986189. Additionally or alternatively, in some embodiments, the machine learning technique is a random forest technique, and the one or more machine learning models are random forest models.

Additionally or alternatively, in some embodiments, the machine learning classifier is an ensemble learning random forest classifier. In certain embodiments, the machine learning classifier is cancer-type specific, and the plurality of features comprises a cancer type.

Additionally or alternatively, in some embodiments, hyperparameter optimization is performed for each cancer type in the plurality of cancer types. In certain embodiments, performing the hyperparameter optimization comprises performing an exhaustive grid search technique.

In any and all embodiments of the computing systems disclosed herein, the plurality of features comprises a plurality of at least one genomic feature, at least one molecular feature, at least one clinical feature, and/or at least one demographic feature. In certain embodiments, the plurality of features comprises at least one genomic feature, at least one molecular feature, at least one clinical feature, and at least one demographic feature. In some embodiments, the plurality of features further comprises a plurality of: (i) a tumor mutation burden (TMB) metric, (ii) a fraction of copy-number alteration (FCNA) metric, (iii) an HLA-I evolutionary divergence (HED) metric, (iv) a loss of heterozygosity (LOH) status in HLA-I, (v) a microsatellite instability (MSI) status, (vi) a body mass index (BMI), (vii) a gender, (viii) a blood neutrophil-to-lymphocyte ratio (NLR) metric, (ix) a tumor stage, (x) an immune checkpoint inhibitor, (xi) an age, (xii) a cancer type, and/or (xiii) an indication of whether chemotherapy has been administered. In other embodiments, the plurality of features comprises: (i) a tumor mutation burden (TMB) metric, (ii) a fraction of copy-number alteration (FCNA) metric, (iii) an HLA-I evolutionary divergence (HED) metric, (iv) a loss of heterozygosity (LOH) status in HLA-I, (v) a microsatellite instability (MSI) status, (vi) a body mass index (BMI), (vii) a gender, (viii) a blood neutrophil-to-lymphocyte ratio (NLR) metric, (ix) a tumor stage, (x) an immune checkpoint inhibitor, (xi) an age, (xii) a cancer type, (xiii) an indication of whether chemotherapy has been administered, (xiv) the blood albumin level, (xv) the blood hemoglobin level, and (xvi) the blood platelet level. The tumor stage may stage I, stage II, Stage III or stage IV.

Additionally or alternatively, in some embodiments, the plurality of features comprises one or more features having discrete values and/or one or more features having continuous values. In some embodiments, the one or more features having discrete values are selected from among an indication of whether chemotherapy has been administered, cancer type, LOH in HLA-I, an immune checkpoint inhibitor, MSI status, and tumor stage. In certain embodiments, the one or more features having continuous values are selected from among a TMB metric, blood albumin level, blood NLR metric, age, blood hemoglobin level, blood platelet level, a FCNA metric, BMI, and a HED metric. Additionally or alternatively, in some embodiments, the plurality of features for the cancer patient are determined by assaying blood and/or sequencing tumor DNA.

In any and all embodiments of the computing systems disclosed herein, the plurality of cancer types are selected from the group consisting of non-small cell lung cancers (NSCLC), small cell lung cancers (SCLC), melanoma, renal cell carcinoma, bladder cancer, head and neck cancer, sarcoma, endometrial cancer, gastric cancer, hepatobiliary cancer, colorectal cancer, esophageal cancer, pancreatic cancer, mesothelioma, ovarian cancer, and breast cancer.

In one aspect, the present disclosure provides a non-transitory computer-readable storage medium comprising instructions which, when executed by a processor of a machine learning system, configure the machine learning system to train a machine learning classifier for predicting responsiveness of cancer patients to an immune checkpoint blockade (ICB) therapy, the instructions configured to cause the processor to: receive data on a cohort of subjects, the subjects in the cohort having a plurality of cancer types; generate a training dataset based on the received data, the training dataset comprising a plurality of features for each subject in the cohort, the plurality of features comprising at least one of a blood albumin level, a blood hemoglobin level, or a blood platelet level; and apply a machine learning method to the training dataset to develop the machine learning classifier for predicting responsiveness of cancer patients to the ICB therapy; wherein applying the machine learning method comprises: applying a machine learning technique to the training dataset to each cancer type individually; performing hyperparameter optimization to identify one or more machine learning models with an accuracy that exceeds an accuracy threshold for the machine learning classifier; and determining an optimal operating-point threshold based on optimization of sensitivity and specificity of the receiver operating characteristic (ROC) curves for the training dataset; and wherein the machine learning classifier is configured to receive the plurality of features for cancer patients and generate predictors for responsiveness of the cancer patients to the ICB therapy. In some embodiments, the machine learning technique is a random forest technique, and the one or more machine learning models are random forest models. The ICB therapy may be a PD-1/PD-L1 inhibitor, a CTLA-4 inhibitor, or a combination thereof. In some embodiments, the ICB therapy comprises one or more of pembrolizumab, nivolumab, cemiplimab, atezolizumab, avelumab, durvalumab, ipilimumab, tremelimumab, ticlimumab, JTX-4014, Spartalizumab (PDR001), Camrelizumab (SHR1210), Sintilimab (IBI308), Tislelizumab (BGB-A317), Toripalimab (JS 001), Dostarlimab (TSR-042, WBP-285), INCMGA00012 (MGA012), AMP-224, AMP-514, KN035, CK-301, AUNP12, CA-170, or BMS-986189.

Additionally or alternatively, in some embodiments, the machine learning classifier is an ensemble learning random forest classifier. In certain embodiments, the machine learning classifier is cancer-type specific, and the plurality of features comprises a cancer type.

Additionally or alternatively, in some embodiments, hyperparameter optimization is performed for each cancer type in the plurality of cancer types. In certain embodiments, performing the hyperparameter optimization comprises performing an exhaustive grid search technique.

In any and all embodiments of the computer-readable storage medium disclosed herein, the plurality of features comprises a plurality of at least one genomic feature, at least one molecular feature, at least one clinical feature, and/or at least one demographic feature. In certain embodiments, the plurality of features comprises at least one genomic feature, at least one molecular feature, at least one clinical feature, and at least one demographic feature. In some embodiments, the plurality of features further comprises a plurality of: (i) a tumor mutation burden (TMB) metric, (ii) a fraction of copy-number alteration (FCNA) metric, (iii) an HLA-I evolutionary divergence (HED) metric, (iv) a loss of heterozygosity (LOH) status in HLA-I, (v) a microsatellite instability (MSI) status, (vi) a body mass index (BMI), (vii) a gender, (viii) a blood neutrophil-to-lymphocyte ratio (NLR) metric, (ix) a tumor stage, (x) an immune checkpoint inhibitor, (xi) an age, (xii) a cancer type, and/or (xiii) an indication of whether chemotherapy has been administered. In other embodiments, the plurality of features comprises: (i) a tumor mutation burden (TMB) metric, (ii) a fraction of copy-number alteration (FCNA) metric, (iii) an HLA-I evolutionary divergence (HED) metric, (iv) a loss of heterozygosity (LOH) status in HLA-I, (v) a microsatellite instability (MSI) status, (vi) a body mass index (BMI), (vii) a gender, (viii) a blood neutrophil-to-lymphocyte ratio (NLR) metric, (ix) a tumor stage, (x) an immune checkpoint inhibitor, (xi) an age, (xii) a cancer type, (xiii) an indication of whether chemotherapy has been administered, (xiv) the blood albumin level, (xv) the blood hemoglobin level, and (xvi) the blood platelet level. The tumor stage may stage I, stage II, Stage III or stage IV.

Additionally or alternatively, in some embodiments, the plurality of features comprises one or more features having discrete values and/or one or more features having continuous values. In some embodiments, the one or more features having discrete values are selected from among an indication of whether chemotherapy has been administered, cancer type, LOH in HLA-I, an immune checkpoint inhibitor, MSI status, and tumor stage. In certain embodiments, the one or more features having continuous values are selected from among a TMB metric, blood albumin level, blood NLR metric, age, blood hemoglobin level, blood platelet level, a FCNA metric, BMI, and a HED metric.

In any and all embodiments of the computer-readable storage medium disclosed herein, the instructions further cause the processor to apply the classifier to data on a cancer patient to generate a predictor, and determine whether the cancer patient is predicted to be responsive to the ICB therapy based on the predictor and the operating-point threshold. The predictor may comprise a response probability value.

In any and all embodiments of the computer-readable storage medium disclosed herein, the plurality of cancer types are selected from the group consisting of non-small cell lung cancers (NSCLC), small cell lung cancers (SCLC), melanoma, renal cell carcinoma, bladder cancer, head and neck cancer, sarcoma, endometrial cancer, gastric cancer, hepatobiliary cancer, colorectal cancer, esophageal cancer, pancreatic cancer, mesothelioma, ovarian cancer, and breast cancer.

In yet another aspect, the present disclosure provides a non-transitory computer-readable storage medium comprising instructions which, when executed by a processor of a computing system, configure the computing system to system for predicting responsiveness of a cancer patient to an immune checkpoint blockade (ICB) therapy, the instructions configured to cause the processor to: receive patient data corresponding to a plurality of features for the cancer patient; apply a machine learning classifier to the patient data to generate a predictor; and determine whether the cancer patient is predicted to be responsive to the ICB therapy based on the predictor and an operating-point threshold, wherein the classifier is trained by: receiving cohort data on a cohort of subjects, the subjects in the cohort having a plurality of cancer types; generating a training dataset based on the received cohort data, the training dataset comprising the plurality of features for each subject in the cohort, the plurality of features comprising at least one of a blood albumin level, a blood hemoglobin level, or a blood platelet level; and applying a machine learning method to the training dataset to develop the machine learning classifier for predicting responsiveness of cancer patients to the ICB therapy, wherein applying the machine learning method comprises: applying a machine learning technique to the training dataset to each cancer type individually; performing hyperparameter optimization to identify one or more machine learning models with an accuracy that exceeds an accuracy threshold for the machine learning classifier; and determining the optimal operating-point threshold based on optimization of sensitivity and specificity of the receiver operating characteristic (ROC) curves for the training dataset; and wherein the machine learning classifier is configured to receive the plurality of features for cancer patients and generate predictors for responsiveness of the cancer patients to the ICB therapy. The ICB therapy may be a PD-1/PD-L1 inhibitor, a CTLA-4 inhibitor, or a combination thereof. In some embodiments, the ICB therapy comprises one or more of pembrolizumab, nivolumab, cemiplimab, atezolizumab, avelumab, durvalumab, ipilimumab, tremelimumab, ticlimumab, JTX-4014, Spartalizumab (PDR001), Camrelizumab (SHR1210), Sintilimab (IBI308), Tislelizumab (BGB-A317), Toripalimab (JS 001), Dostarlimab (TSR-042, WBP-285), INCMGA00012 (MGA012), AMP-224, AMP-514, KN035, CK-301, AUNP12, CA-170, or BMS-986189. Additionally or alternatively, in some embodiments, the predictor comprises a response probability value. In certain embodiments, the machine learning technique is a random forest technique, and the one or more machine learning models are random forest models.

Additionally or alternatively, in some embodiments, the machine learning classifier is an ensemble learning random forest classifier. In certain embodiments, the machine learning classifier is cancer-type specific, and the plurality of features comprises a cancer type.

Additionally or alternatively, in some embodiments, hyperparameter optimization is performed for each cancer type in the plurality of cancer types. In certain embodiments, performing the hyperparameter optimization comprises performing an exhaustive grid search technique.

In any and all embodiments of the computer-readable storage medium disclosed herein, the plurality of features comprises a plurality of at least one genomic feature, at least one molecular feature, at least one clinical feature, and/or at least one demographic feature. In certain embodiments, the plurality of features comprises at least one genomic feature, at least one molecular feature, at least one clinical feature, and at least one demographic feature. In some embodiments, the plurality of features further comprises a plurality of: (i) a tumor mutation burden (TMB) metric, (ii) a fraction of copy-number alteration (FCNA) metric, (iii) an HLA-I evolutionary divergence (HED) metric, (iv) a loss of heterozygosity (LOH) status in HLA-I, (v) a microsatellite instability (MSI) status, (vi) a body mass index (BMI), (vii) a gender, (viii) a blood neutrophil-to-lymphocyte ratio (NLR) metric, (ix) a tumor stage, (x) an immune checkpoint inhibitor, (xi) an age, (xii) a cancer type, and/or (xiii) an indication of whether chemotherapy has been administered. In other embodiments, the plurality of features comprises: (i) a tumor mutation burden (TMB) metric, (ii) a fraction of copy-number alteration (FCNA) metric, (iii) an HLA-I evolutionary divergence (HED) metric, (iv) a loss of heterozygosity (LOH) status in HLA-I, (v) a microsatellite instability (MSI) status, (vi) a body mass index (BMI), (vii) a gender, (viii) a blood neutrophil-to-lymphocyte ratio (NLR) metric, (ix) a tumor stage, (x) an immune checkpoint inhibitor, (xi) an age, (xii) a cancer type, (xiii) an indication of whether chemotherapy has been administered, (xiv) the blood albumin level, (xv) the blood hemoglobin level, and (xvi) the blood platelet level. The tumor stage may stage I, stage II, Stage III or stage IV.

Additionally or alternatively, in some embodiments, the plurality of features comprises one or more features having discrete values and/or one or more features having continuous values. In some embodiments, the one or more features having discrete values are selected from among an indication of whether chemotherapy has been administered, cancer type, LOH in HLA-I, an immune checkpoint inhibitor, MSI status, and tumor stage. In certain embodiments, the one or more features having continuous values are selected from among a TMB metric, blood albumin level, blood NLR metric, age, blood hemoglobin level, blood platelet level, a FCNA metric, BMI, and a HED metric. In certain embodiments, the plurality of features for the cancer patient are determined by assaying blood and/or sequencing tumor DNA.

In any and all embodiments of the computer-readable storage medium disclosed herein, the plurality of cancer types are selected from the group consisting of non-small cell lung cancers (NSCLC), small cell lung cancers (SCLC), melanoma, renal cell carcinoma, bladder cancer, head and neck cancer, sarcoma, endometrial cancer, gastric cancer, hepatobiliary cancer, colorectal cancer, esophageal cancer, pancreatic cancer, mesothelioma, ovarian cancer, and breast cancer.

### EXAMPLES

The present technology is further illustrated by the following Examples, which should not be construed as limiting in any way.

### Example 1: Experimental Methods

### Patient Data Description

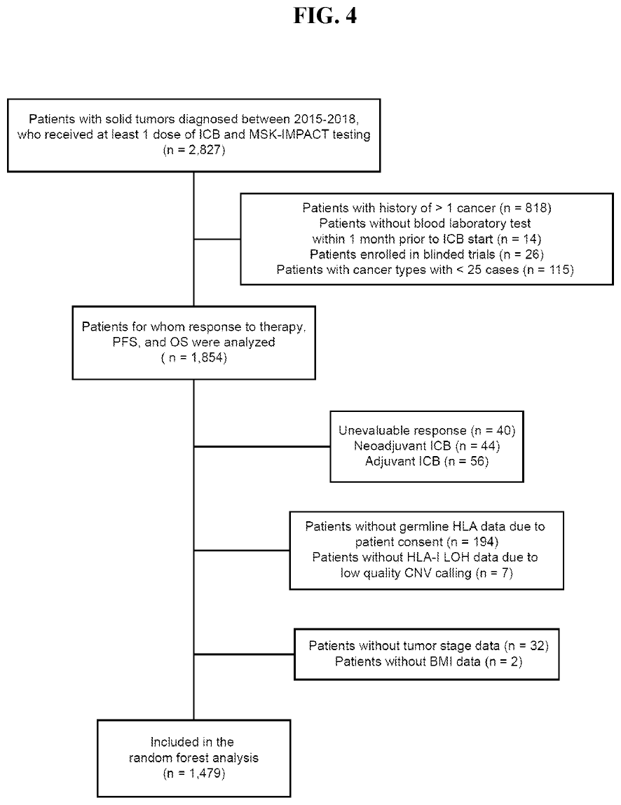

The use of the patient data was approved by the MSKCC Institutional Review Board (IRB). All patients provided informed consent to an institutional review board-approved protocol. The main study question (whether the integrated model disclosed herein could predict response to immunotherapy, OS, and PFS) was specified before data collection began. Potential immortal time bias due to left truncation was addressed by limiting the cohort to patients followed after receiving a cancer diagnosis during the period when tumor sequencing was routinely performed. Patients initially selected for this study were those with solid tumors diagnosed from 2015 through 2018 who received at least 1 dose of ICB (n=2,827). All tumors, along with DNA from peripheral blood, were genomically profiled using the MSK-IMPACT next-generation sequencing platform (CLIA-approved hybridization-capture based assay) (Zehir, A. et al. Nat Med 23, 703-713 (2017)). Patients with a history of more than 1 cancer, those without a complete blood count within 30 days prior to the first dose of ICB, those enrolled in blinded trials, and cancer types with fewer than 25 cases were excluded. The clinical records of the remaining 1,854 patients were manually reviewed to evaluate response to therapy, OS, and PFS. The process was blinded to patients' genomic, molecular, and clinical data. Patients who received ICB in a neoadjuvant or adjuvant setting, and patients with unevaluable response (lost to follow-up without imaging after ICB start) were excluded. Patients without HLA data due to consent for germline testing or poor HLA genotyping quality were also excluded. Patients without stage and BMI information were further excluded. The final set consisted of 1,479 patients from 16 cancer types (FIG. 4).

### Response, Overall Survival, and Progression-Free Survival

The primary study outcomes were response to ICB, OS, and PFS. Response based on RECIST v1.1 criteria (Eisenhauer, E. A. & Verweij, J. Ejc Suppl 7, 5-5 (2009)) was categorized. If formal RECIST reads were not available, physician notes and imaging studies were manually reviewed to categorize overall best response for each patient using the same criteria based on change in the sum of diameters of target lesions. CR and PR were classified as responders; and SD and PD were classified as non-responders. PFS was calculated from ICB first infusion to disease progression or death of any cause; patients without progression were censored at last attended appointment at MSKCC with any clinician. OS was calculated from ICB first infusion to death of any cause; patients alive at time of review were censored at last contact. For patients who received multiple lines of ICB, the first line was used for analysis.

### Genomic, Demographic, Molecular, and Clinical Data

Blood neutrophil-to-lymphocyte ratio was calculated as the absolute count of neutrophils (per nL) divided by the absolute count of lymphocytes (per nL). The units for albumin and HGB were g/dL, and platelets per nL. All peripheral blood values were gathered from the closest blood test prior to the first ICB infusion (all within a month prior to ICB start). BMI was calculated by dividing patients' body weight (kg) over the square of height (m²) assessed before ICB treatment. TMB was measured as the total number of somatic tumor non-synonymous mutations normalized to the exonic coverage of the respective MSK-IMPACT panel in megabases (mut/Mb) (Samstein, R. M. et al. Nature Genetics 51, 202-206 (2019)). The MSK-IMPACT panel identifies non-synonymous mutations in 468 genes (earlier versions included 341 or 410 genes). For tumor-derived genomic data, the MSK-IMPACT performed from the earliest retrieved sample was used if a patient had more than one MSK-IMPACT test. Clinical and demographic variables incorporated in the model were age at ICB first infusion, sex, cancer type, ICB drug class, tumor stage at ICB first infusion, and history of chemotherapy prior to ICB treatment start. Cancers were staged according to the American Joint Committee on Cancer (AJCC) 8th Edition (Amin, M. B. et al. C A Cancer J Clin 67, 93-99 (2017)). FCNA was calculated as the length of FACETS (Zhou, J. et al. Clinical Cancer Research 25, 7475-7484 (2019); Shen, R. L. & Seshan, V. E. Nucleic Acids Research 44, e131) segments with |cnlr.median.clust|>=0.2 (i.e. segments with log 2 CNA value >0.2) divided by the total length of all segments. FACETS segments were classified as LOH if they had total copy number (tcn) >=2 and minor allele copy number (lcn)=0. HLA-I loci were classified as LOH if they overlapped (by any amount) an LOH segment. Segments with tcn=1 and lcn=0 were considered hemizygous, not LOH. MSI status of each tumor was determined by MSIsensor (Niu, B. F. et al. Bioinformatics 30, 1015-1016, doi:10.1093/bioinformatics/btt755 (2014)) with following criteria: stable (0≤MSI score<3), indeterminate (3≤MSI score<10), and unstable (10≤MSI score). In the machine learning model, two groups for MSI status: MSI unstable vs. MSI stable/indeterminate were used.

High-resolution HLA-I genotyping from germline normal DNA sequencing data was performed. For each patient, the most recent MSK-IMPACT targeted gene panel was obtained and Polysolver was used to identify HLA-I alleles with default parameter settings (Shukla, S. A. et al. Nat Biotechnol 33, 1152-1158, doi:10.1038/nbt.3344 (2015)). For quality assurance of HLA-I genotyping using MSK-IMPACT, HLA-I typing by Polysolver between 37 sequenced samples was compared with MSK-IMPACT and whole exome. The MSK-IMPACT panel successfully captured HLA-A, -B, and -C reads and validation was previously performed (Chowell, D. et al. Science 359, 582-587 (2018)). The overall concordance of HLA-I typing between the MSK-IMPACT samples and their matched whole exome sequenced (WES) samples was 96%. To ensure that HLA-I genes had adequate coverage in MSK-IMPACT bam files, bedtools multicov tool (Quinlan, A. R. & Hall, I. M. Bioinformatics 26, 841-842, (2010)) was also applied. The bedtools multicov tool reports the count of alignments from multiple position-sorted and indexed BAM files that overlap with targets intervals in a BED format. Only high-quality reads were counted and only samples with sufficient coverage were used.

HED was calculated as previously described in Pierini, F. & Lenz, T. L. Mol Biol Evol, doi:10.1093/molbev/msy116 (2018) and Chowell, D. et al. Nat Med 25, 1715-1720, (2019), both of which are incorporated herein by reference. Briefly, the protein sequence of exons 2 and 3 of each allele of each patient's HLA-I genotype, which correspond to the peptide-binding domains were extracted. Protein sequences were obtained from the IMGT/HLA database (Robinson, J. et al. Nucleic Acids Research 43, D423-D431 (2015)), and exons coding for the variable peptide-binding domains were selected following the annotation obtained from Ensembl database (Zerbino, D. R. et al. Nucleic Acids Res 46, D754-D761 (2018)). Divergence values between allele sequences were calculated using the Grantham distance metric, as implemented in Pierini, F. & Lenz, T. L. Mol Biol Evol, doi:10.1093/molbev/msy116 (2018), which is incorporated herein by reference. The Grantham distance is a quantitative pairwise distance in which the physiochemical properties of amino acids, and hence the functional similarity between sequences are considered (Grantham, R. Science 185, 862-864 (1974), which is incorporated herein by reference). Given a particular HLA-I locus with two alleles, the sequences of the peptide-binding domains of each allele are aligned (Edgar, R. C. Nucleic Acids Res 32, 1792-1797 (2004)), and the Grantham distance is calculated as the sum of amino acid differences (taking into account the biochemical composition, polarity, and volume of each amino acid) along the sequences of the peptide-binding domains, following the formula by R. Grantham (Grantham, R. Science 185, 862-864 (1974)):

$$Grantham Distance = \sum {D}_{ij} = \sum {[ {α ( {c}_{i} - {c}_{j} )}^{2} + {β ( {p}_{i} - {p}_{j} )}^{2} + {γ ( {v}_{i} - {v}_{j} )}^{2} ]}^{1 / 2} \qquad ( 1 )$$

where i and j are the two homologous amino acids at a given position in the alignment, c, p, and v represent composition, polarity, and volume of the amino acids respectively, and α, β, and γ are constants; all values are taken from the original study (Grantham, R. Science 185, 862-864 (1974)). The final Grantham distance is calculated by normalizing the value from (1) by the length of the alignment between the peptide-binding domains of a particular HLA-I genotype's two alleles. In the model, HED was referred as mean HED which was calculated as the mean of divergences at HLA-A, HLA-B, and HLA-C.

### Model Description

A random forest classifier using scikit-learn package (Pedregosa, F. et al. J Mach Learn Res 12, 2825-2830 (2011)) was implemented in Python 3.0 programming language (www.python.org/). To generate the training (80%) and test (20%) datasets, the dataset was split using the train_test_split function which randomly partitions a dataset into training and test subsets with test_size=0.2 parameter. This function was applied to each cancer type individually. To build a random forest classifier with best hyperparameters, the exhaustive grid search approach using GridSearchCV function was implemented to the training dataset with 5-fold cross-validation. Model features were used as discrete values (chemotherapy prior ICB, cancer type, LOH in HLA-I, drug class, MSI status, and tumor stage) or continuous values (TMB, albumin, NLR, age, HGB, platelets, FCNA, BMI, and HED). A total 10,000 random forest classifier models were evaluated with different combinations of hyperparameters: max_features=“auto”; n_estimators ranging from 100 to 1000 with an interval of 100; max_depth ranging from 2 to 20 with an interval of 2; min_samples_leaf ranging from 2 to 20 with an interval of 2; min_samples_split ranging from 2 to 20 with an interval of 2. As a result, a model with n_estimators=1000, max_depth=8, min_samples_leaf=20, and min_samples_split=2 hyperparameters which showed the highest average accuracy at 0.7559 was selected. Also, the RF11 model was built with n_estimators=300, max_depth=4, min_samples_leaf=12, and min_samples_split=2 hyperparameters which showed the highest average accuracy at 0.7576. To compute the feature importance for the RF16 model, the permutation-based importance (function PermutationImportance) from the ELI5 Python package (eli5.readthedocs.io/) was used.

### Logistic Regression Analysis

The glm function was used for the logistic regression model. The stepAIC function from the MASS package was applied to model selection, and the predict function was used to get the response probability of samples in the test set. To compute standardized coefficients, the beta function from the reghelper package was used. All the analyses were performed using R programming language (www.r-project.org/).

### Optimal Probability Threshold

The optimal threshold of the probabilities of response were computed by the random forest and the logistic regression models, which discriminates responder and non-responder by using the Youden's index method using the pROC package (Robin, X. et al. BMC Bioinformatics 12, 77 (2011)). The optimal threshold was also determined with the highest F-score, and there was no significant difference in the predictive power of the thresholds using Youden's index or F-score.

### Statistical Analyses

To compare the distributions of response probability generated by the integrated model vs. TMB between response groups, the two-sided Mann-Whitney U test was used. The correlation coefficients between the response probabilities from the pan-cancer and cancer-specific models were calculated by Spearman's rank test. The C-index of the model predictions and TMB alone was calculated by concordance.index function and they were compared using cindex.comp function which implements a paired Student t-test from the survcomp package (Schroder, M. S., Culhane, A. C., Quackenbush, J. & Haibe-Kains, B. Bioinformatics 27, 3206-3208 (2011)). Brier score was calculated using the pec package (Mogensen, U. B., Ishwaran, H. & Gerds, T. A. J Stat Softw 50, 1-23 (2012)). ROC and precision-recall curves were visualized and the area under the curve was calculated using the precrec package (Saito, T. & Rehmsmeier, M. Bioinformatics 33, 145-147 (2017)). The Kaplan-Meier plot, log-rank P-value, and Cox proportional hazard ratio (HR) were generated by the survminer package. All the aforementioned packages and functions are included in R programming language (www.r-project.org/).

### Code Availability

The codes used in this study may be accessed at https://github.com/CCF-ChanLab/MSK-IMPACT-IO.

### Example 2: The Integrated Model of the Present Technology Accurately Predicts Immune Checkpoint Blockade Efficacy Across Multiple Cancer Types

The aim of this study was to develop a machine learning algorithm to generate an accurate prediction of a patient's probability of positive immunotherapy response by comprehensively integrating multiple biological features associated with immunotherapy efficacy, and to assess their individual contribution to response when combined in a single predictive framework.

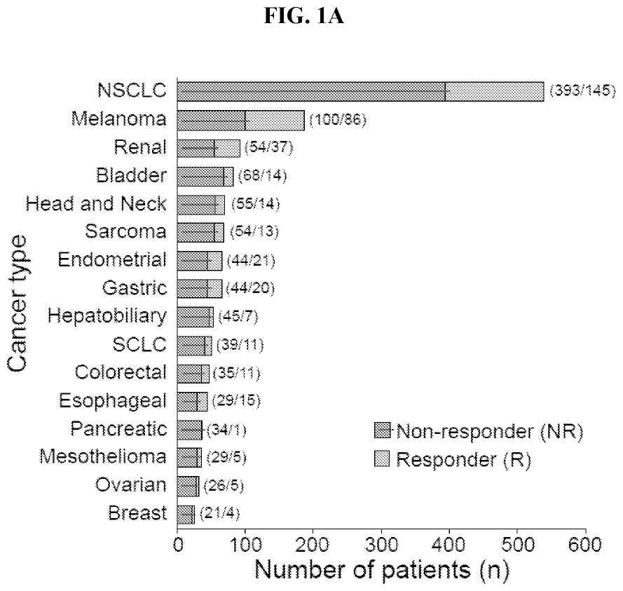

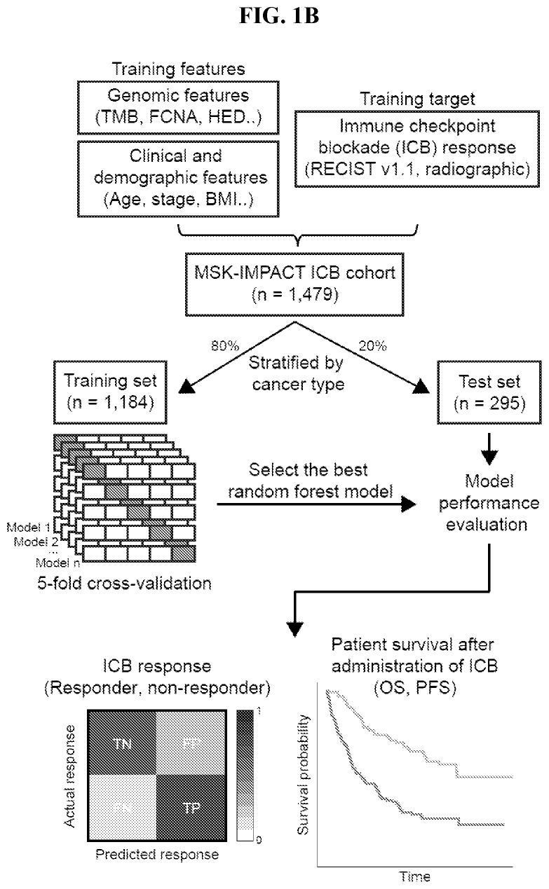

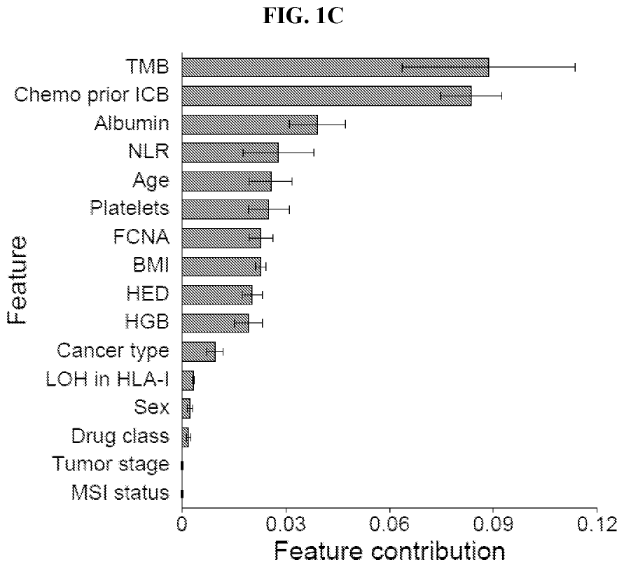

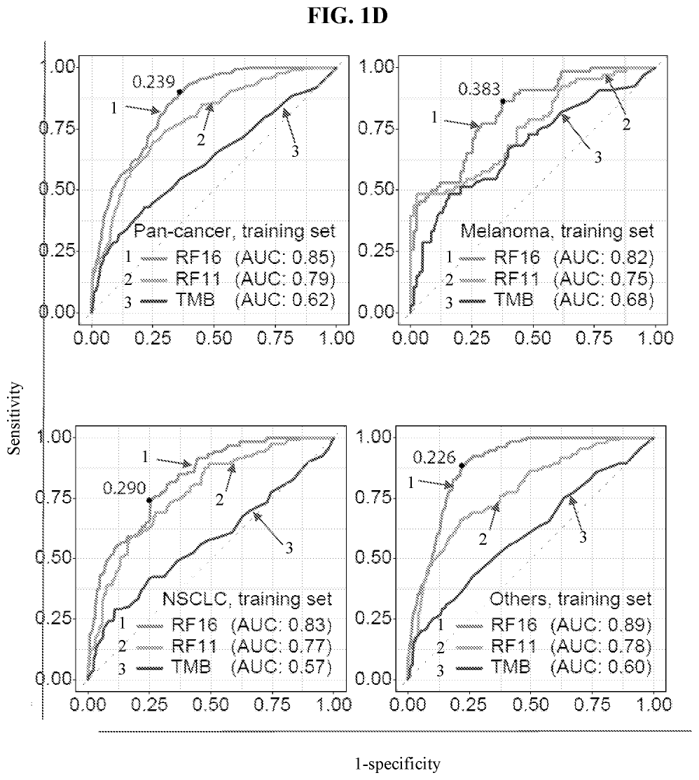

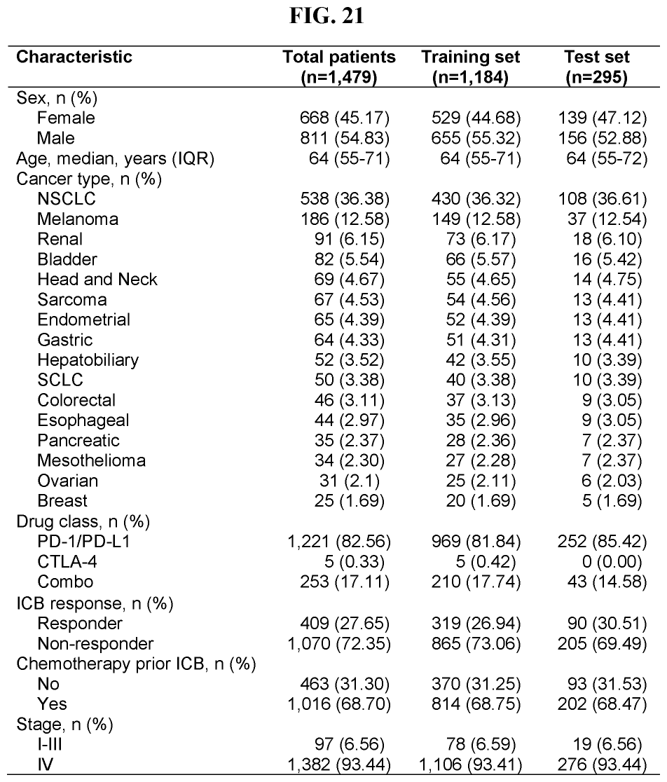

Complete clinical, tumor, and normal sequencing data of 1,479 patients across 16 different cancer types (FIG. 1A and FIG. 4) was acquired. Approximately 37% of the patients had non-small cell lung cancers (NSCLC), 13% had melanomas, and the remaining 50% had other cancer types (hereafter referred to as others), including renal cell carcinoma, bladder, head and neck, and colorectal cancer (FIG. 1A and FIG. 21). These patients were treated with PD-1/PD-L1 inhibitors, CTLA-4 blockade, or combination of both immunotherapy agents (FIG. 21). In total, there are 409 patients whose tumors responded to the immunotherapy and 1,070 patients whose tumors did not respond across the different cancers (FIG. 1A and FIG. 21). Response was based on RECIST v1.1 criteria or best overall response on imaging (Methods). Patients who experienced complete response (CR) or partial response (PR) were classified as responders (R); and patients who experienced stable disease (SD) or progressive disease (PD) were classified as non-responders (NR). Patients' tumors were profiled using the FDA-cleared MSK-IMPACT next-generation sequencing platform (Methods).

To calculate the probability of response to immunotherapy, an ensemble learning random forest (Breiman, L. Random forests. Mach Learn 45, 5-32 (2001)) classifier with 16 input features (hereafter named RF16) were developed. Genomic, molecular, clinical, and demographic variables were incorporated in the model, some previously reported to be associated with ICB response. The variables incorporated were: TMB, fraction of copy-number alteration (FCNA) (Davoli, T., Uno, H., Wooten, E. C. & Elledge, S. J. Science 355, doi:10.1126/science.aaf8399 (2017)), HLA-I evolutionary divergence (HED) (Chowell, D. et al. Nat Med 25, 1715-1720 (2019)), loss of heterozygosity (LOH) status in HLA-I (Chowell, D. et al. Science 359, 582-587 (2018)), microsatellite instability (MSI) status (Mandal, R. et al. Science 364, 485-491 (2019); Le, D. T. et al. Science, doi:10.1126/science.aan6733 (2017)), body mass index (BMI) (Wang, Z. et al. Nat Med 25, 141-151, (2019); Sanchez, A. et al. Lancet Oncol 21, 283-293, (2020)), sex (Conforti, F. et al. Lancet Oncol 19, 737-746 (2018)), blood neutrophil-to-lymphocyte ratio (NLR) (Jaillon, S. et al. Nat Rev Cancer 20, 485-503 (2020); Li, M. J. et al. J Cancer Res Clin 145, 2541-2546, doi:10.1007/s00432-019-02982-4 (2019); Valero, C. et al. Nat Commun 12, 729 (2021)), tumor stage (Kuai, J., Yang, F., Li, G. J., Fang, X. J. & Gao, B. Q. Onco Targets Ther 9, 3763-3770 (2016)), immunotherapy drug agent (Samstein, R. M. et al. Nature Genetics 51, 202-206 (2019)), and age (Ikeguchi, A., Machiorlatti, M. & Vesely, S. K. Melanoma Manag 7, MMT43, doi:10.2217/mmt-2020-0002 (2020)) (Methods). Additionally, cancer type, whether the patient received chemotherapy before immunotherapy, and blood levels of albumin, platelets, and hemoglobin (HGB) were included (Jurasz, P., Alonso-Escolano, D. & Radomski, M. W. Br J Pharmacol 143, 819-826 (2004); Gupta, D. & Lis, C. G. Nutr J 9, doi:Artn 6910.1186/1475-2891-9-69 (2010); Caro, J. J., Salas, M., Ward, A. & Goss, G. Cancer 91, 2214-2221 (2001)) (FIG. 1B).

The dataset was randomized by cancer type into a training subsample (80%, n=1,184) (FIG. 1B and FIG. 21), for which the prediction algorithm was developed, and a test subsample (20%, n=295), on which the trained classifier was evaluated (FIG. 1B and FIG. 21). 5-fold cross-validation was used on the training data to derive the ICB response predictive model based on binary classification (responder and non-responder) (FIG. 1B).

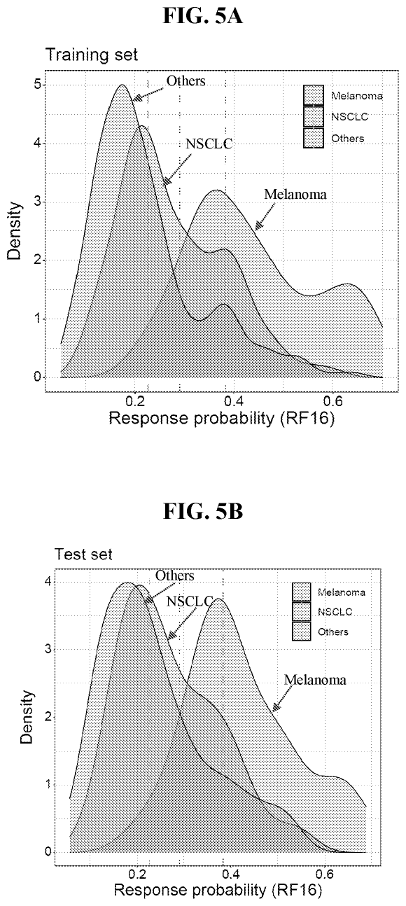

The resulting trained model aggregates the predictive effects across the selected clinical, molecular, demographic, and genomic features to derive a cancer type-specific probability of immunotherapy response. By using this type of model, the extent to which the various features contribute to explaining patient-to-patient variation in response can be quantified (FIG. 1C). These estimates represent the contributions of the various categories of predictors to response outcomes at the population level. At the individual level, each patient was scored based on their response probability (higher values indicate higher probability of ICB response) (FIGS. 5A-5B).

When comparing single model feature contributions of response prediction, TMB was the predictor exerting the greatest effect (FIG. 1C). It was further found that specific correlates of ICB response such as NLR, FCNA, BMI, and HED were ranked by the model as among the top 10 predictive features (FIG. 1C). Notably, MSI status was not selected by the model as one of the top predictors across cancers due to the relatively small percentage of MSI unstable tumors (3.04%) across all cancer types and perhaps due to its association with TMB (Middha, S. et al. Jco Precis Oncol 1, doi:10.1200/Po.17.00084 (2017)). In addition, new strong molecular predictors such as levels of albumin, HGB, and platelets were identified, and their relative contribution to ICB response was quantified (FIG. 1C).

The performance of the integrated clinical-genetic model using multiple metrics was evaluated (Harrell, F. E., Jr., Lee, K. L. & Mark, D. B. Statistics in medicine 15, 361-387, doi:10.1002/(SICI)1097-0258(19960229)15:4<361::AID-SIM168>3.0.CO; 2-4 (1996); Steyerberg, E. W. et al. Epidemiology 21, 128-138, doi:10.1097/EDE.0b013e3181c30fb2 (2010)). To assess the predictive power of integrating the cancer type, whether the patient received chemotherapy before immunotherapy, and the blood markers (albumin, HGB, and platelets), with the other variables that influence ICB response, a second random forest model (hereafter called RF11), including only the variables FCNA, TMB, HED, NLR, BMI, LOH in HLA-I, sex, age, MSI status, tumor stage, and drug class, was developed. The RF11 model was used as a reference for the RF16 model to determine the added value of including the additional variables (FIG. 1C). Since TMB has been approved by the FDA as a biomarker to predict ICB efficacy in solid tumors, the performance of the integrated RF16 and RF11 models was also compared with predictions based on TMB alone.

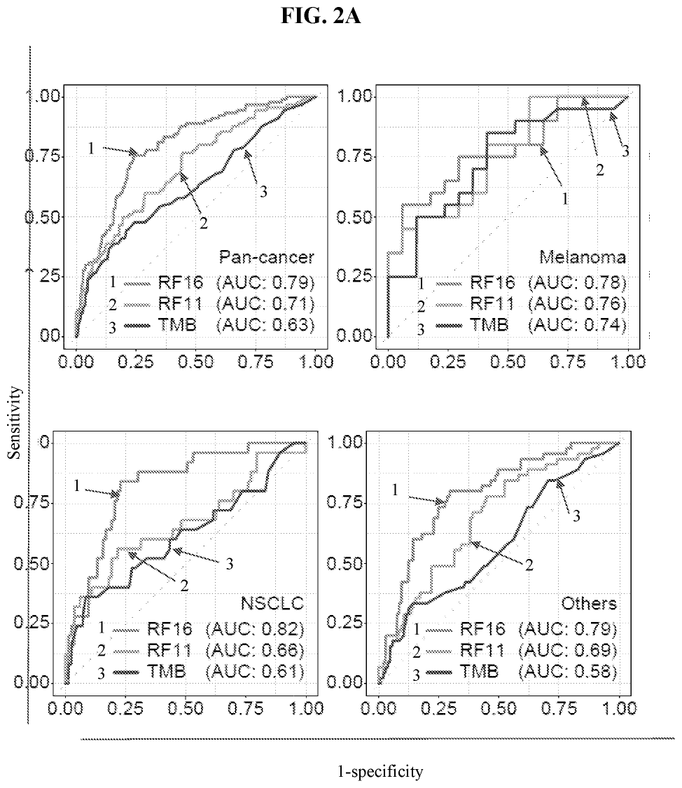

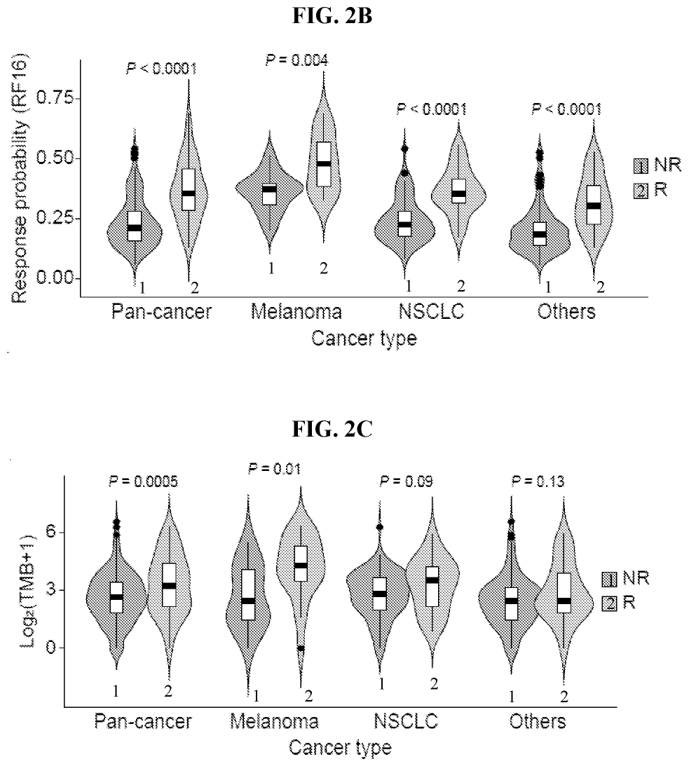

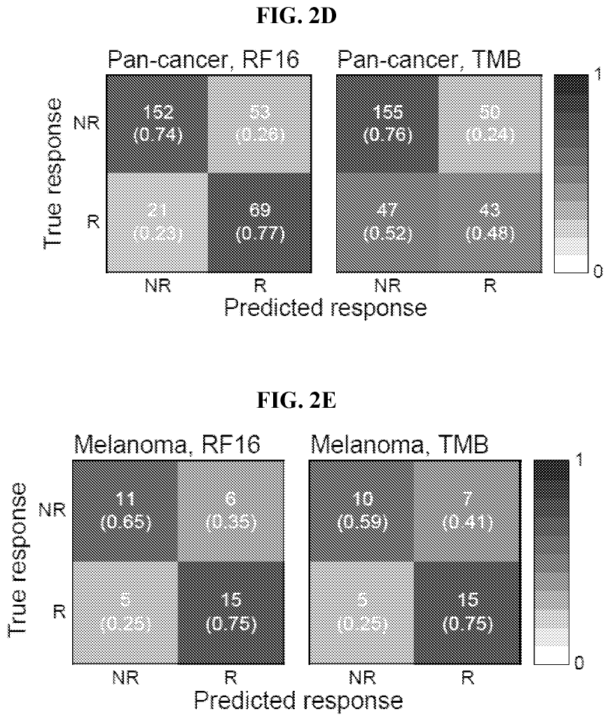

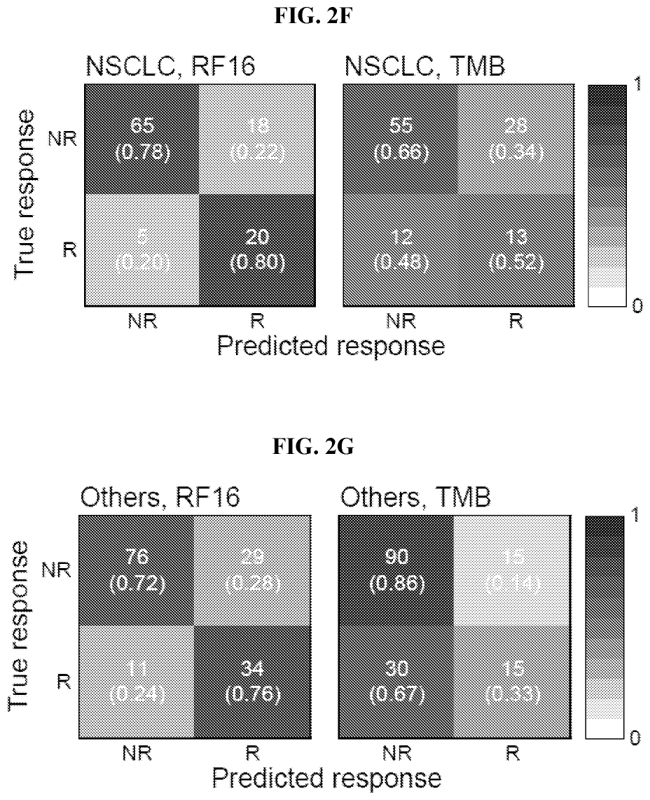

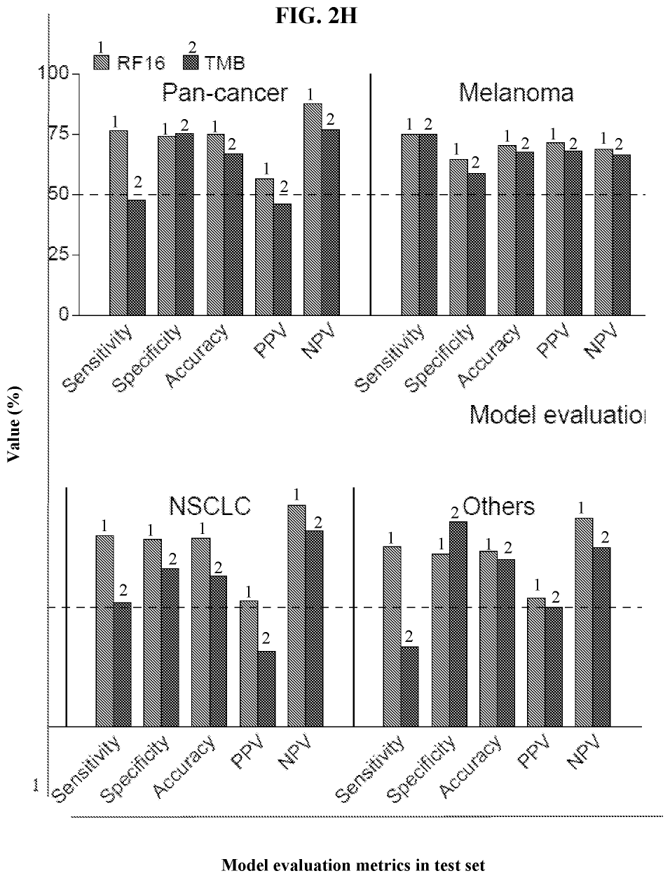

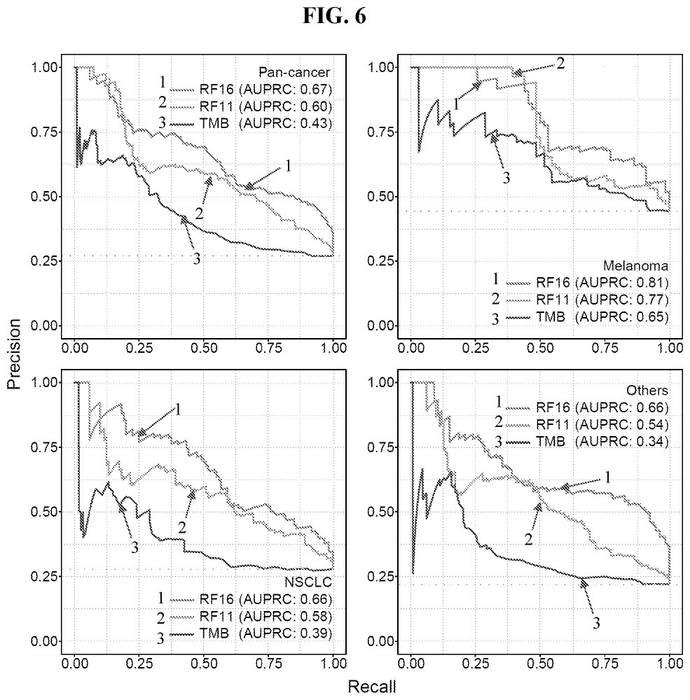

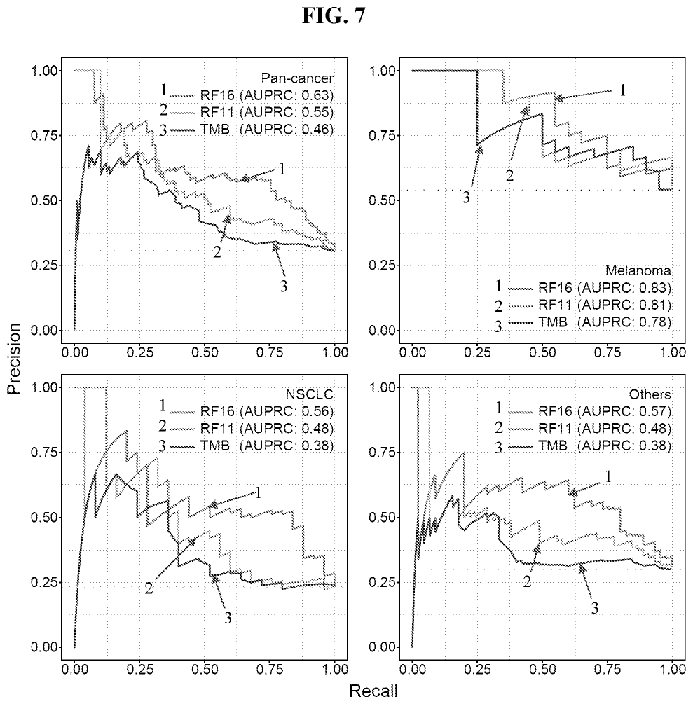

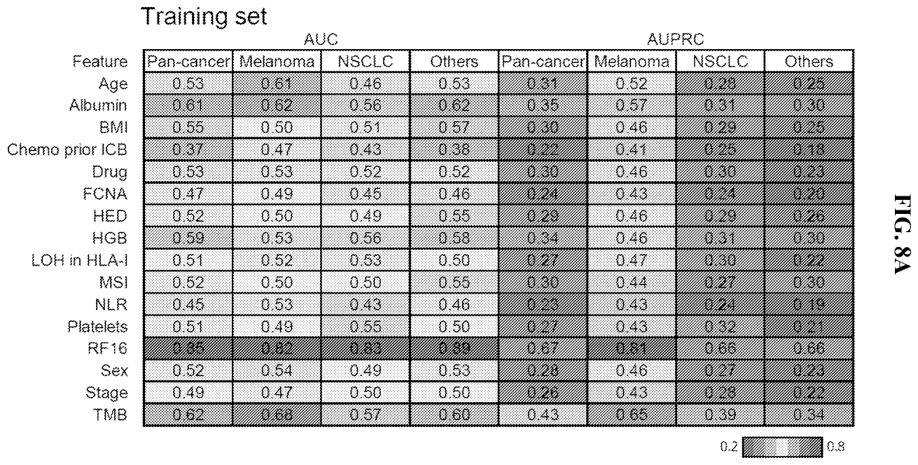

The area under the receiver-operating characteristic (ROC) and precision-recall curves was first calculated by using the response probabilities computed by the respective RF16 and RF11 models and the continuous values of TMB. The integrated RF16 model achieved superior performance as indicated by area under the curve (AUC) in predicting responders and non-responders across cancer types compared to TMB alone and the RF11 model in both the training set (FIG. 1D; pan-cancer AUC 0.85 for RF16 versus 0.79 for RF11 versus 0.62 for TMB) and the test set (FIG. 2A; pan-cancer AUC 0.79 for RF16 versus 0.71 for RF11 versus 0.63 for TMB). Higher area under precision-recall curve (AUPRC) was achieved by the RF16 model than TMB alone and RF11 in both the training (FIG. 6) and the test (FIG. 7) sets. Importantly none of RF16's features alone could achieve the level of performance achieved by RF16, suggesting that each of the model features contributed to the overall prediction performance (FIGS. 8A-8B). Additionally, the continuous probabilities calculated by the integrated RF16 model were significantly associated with response across tumors in the test set (FIG. 2B). Importantly, the differences in response probability between responders and non-responders with RF16 were significantly higher compared to differences in TMB between responder and non-responder groups across the various cancers (FIGS. 2B-2C; pan-cancer P<0.0001 and P=0.0005 for RF16 and TMB, respectively).

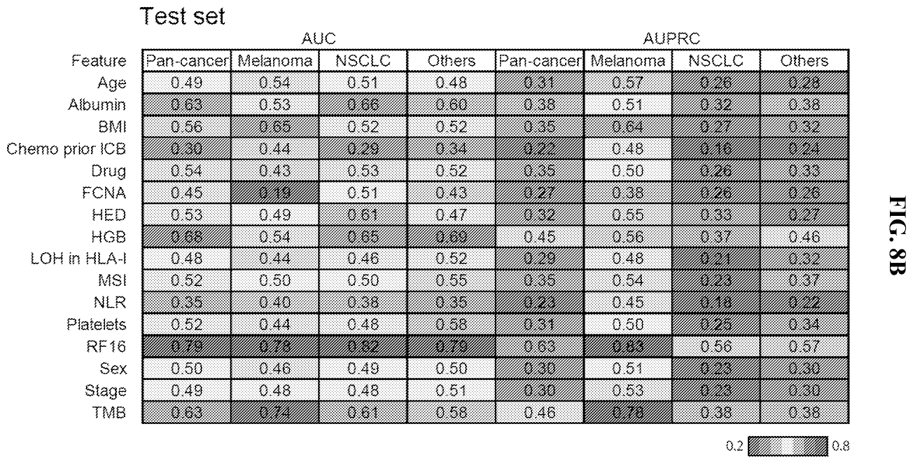

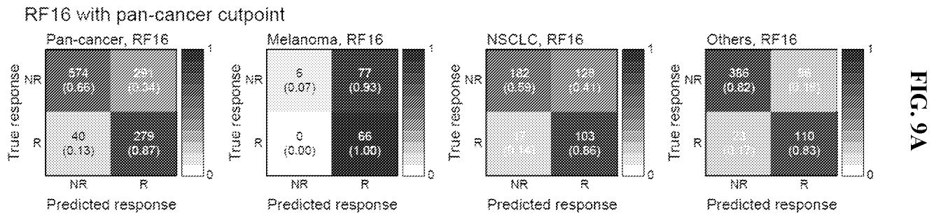

In order to stratify the continuous probabilities generated by RF16 into predicted responder and non-responder groups, the probability that optimizes the sensitivity and specificity of the ROC curves in the training set was determined (FIG. 1D). When the probability value exceeds the optimal operating-point threshold, the tumor would be considered a ‘predicted responder’. Importantly, it was observed that the probability distributions significantly varied across tumor types in the training set (FIG. 5A). Therefore, a single pan-cancer cut-point to discriminate responders and non-responders would result in a high false positive rate in the training set, particularly in melanoma and NSCLC (FIG. 9A). The probabilities were thus dichotomized into predicted responder and non-responder groups by optimizing the sensitivity and specificity in the training set for each cancer group (melanoma, NSCLC, and others) separately (FIG. 1D and FIG. 5A), which reduced false positive predictions in melanoma and NSCLC significantly (FIG. 9B). To test the discriminatory power of these optimal cancer-specific cut-points, they were applied to each cancer group of the test set (FIG. 2D-2G). To compare the performance of predicting responders and non-responders by RF16 with TMB alone, ≥10 mutations per megabase (mut/Mb) was used as the cutpoint for TMB which was approved by the FDA for pembrolizumab. It was found that the RF16 model consistently achieved higher predictive performance as measured by sensitivity, specificity, accuracy, positive predictive value (PPV), and negative predictive value (NPV) compared to TMB alone (FIG. 2H and FIGS. 9C-9D). In particular, the RF16 model achieved significantly higher sensitivity than TMB in NSCLC (80.00% for RF16 vs. 52.00% for TMB) and other cancer types (75.56% for RF16 vs. 33.33% for TMB) in the test set (FIG. 2H). In a pan-cancer analysis, the RF16 model achieved 76.67% sensitivity and 74.15% specificity compared to 47.78% sensitivity and 75.61% specificity achieved by TMB alone in the test set (FIG. 2H). Taken together, our integrated RF16 model predicts response to ICB therapy with high accuracy, as shown by various common performance metrics across different cancer types.

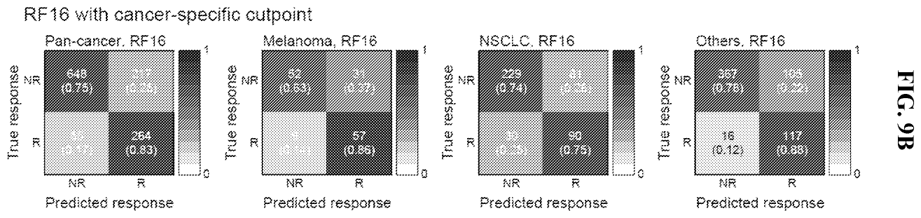

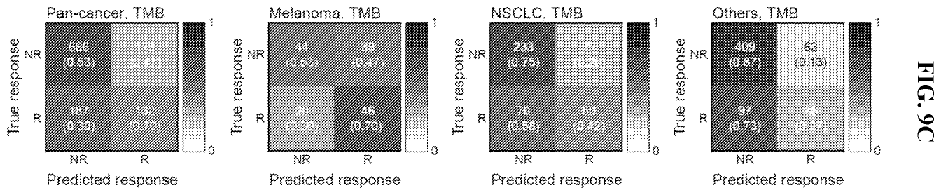

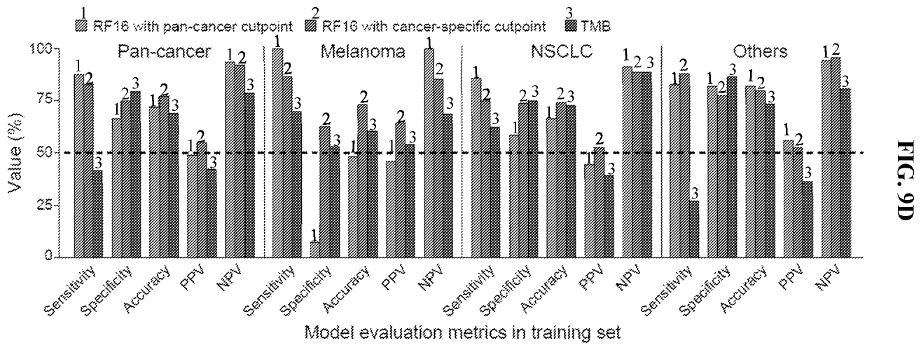

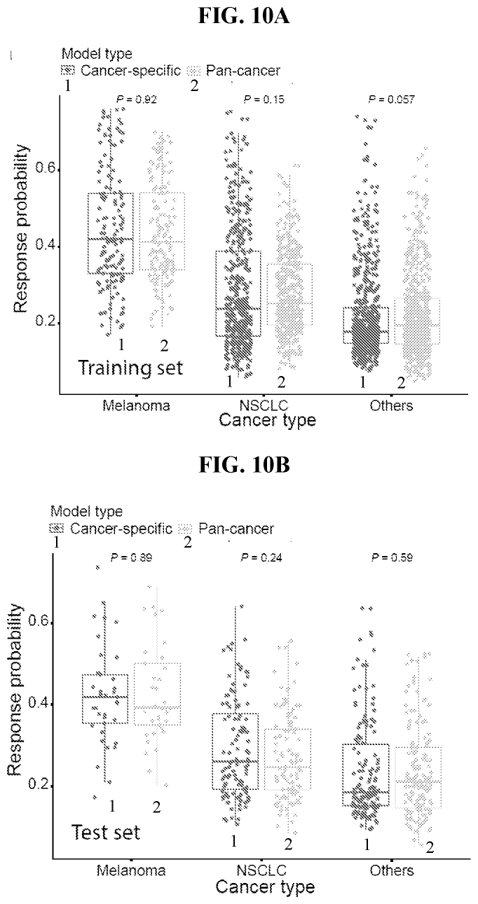

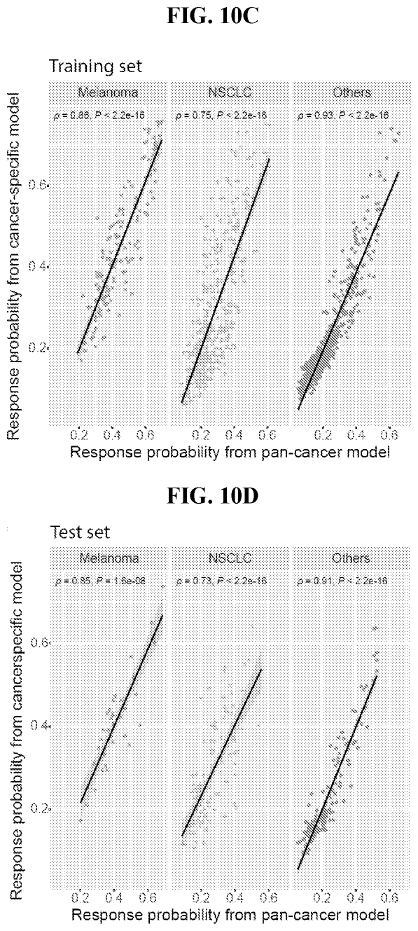

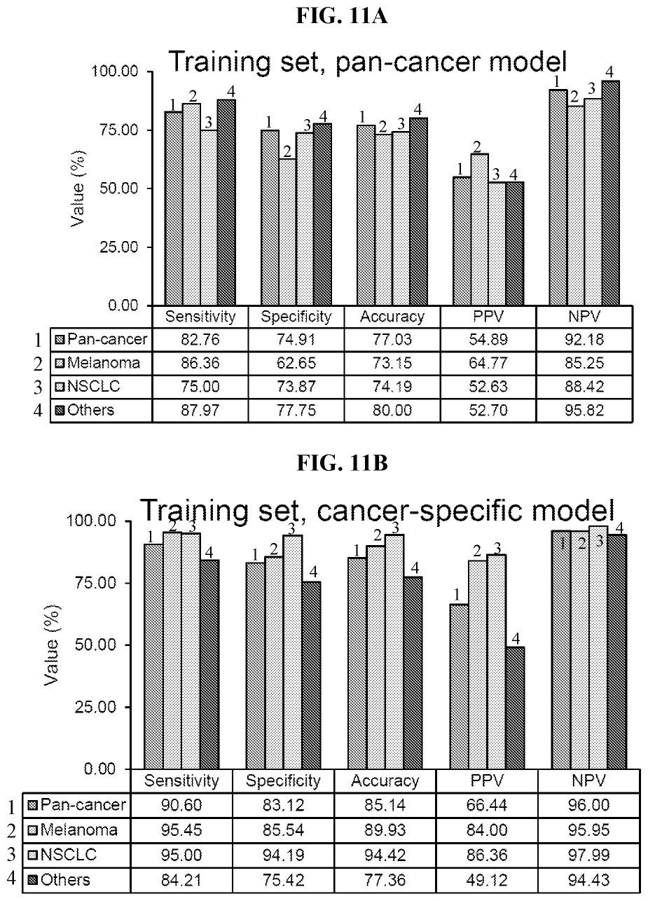

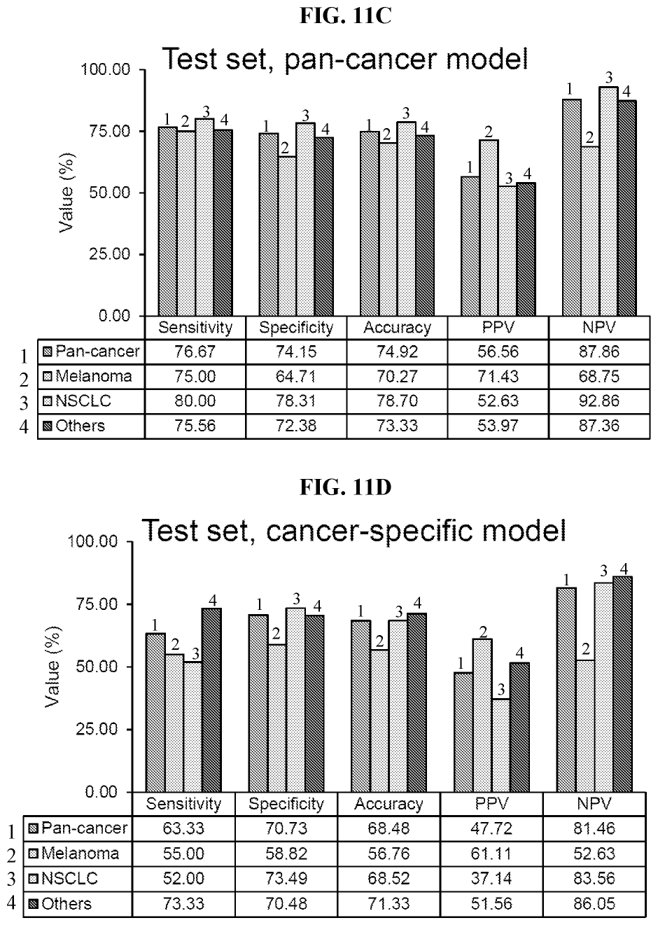

Additionally, the distributions of response probabilities generated from the RF16 model trained on pan-cancer data were compared with those from separate models trained on cancer-specific data. These response probabilities distributions were statistically similar in both the training and test sets (FIGS. 10A-10D). Importantly, the RF16 model trained on the pan-cancer data achieved higher predictive performance compared to the RF16 model trained on cancer-specific data in the test set (FIG. 11A-11D). Thus, these results suggest that the RF16 trained on the large pan-cancer data was able to both learn cancer-specific relationships and generalize relationships that could be relevant across cancers, leading to higher predictive performance in the test set.

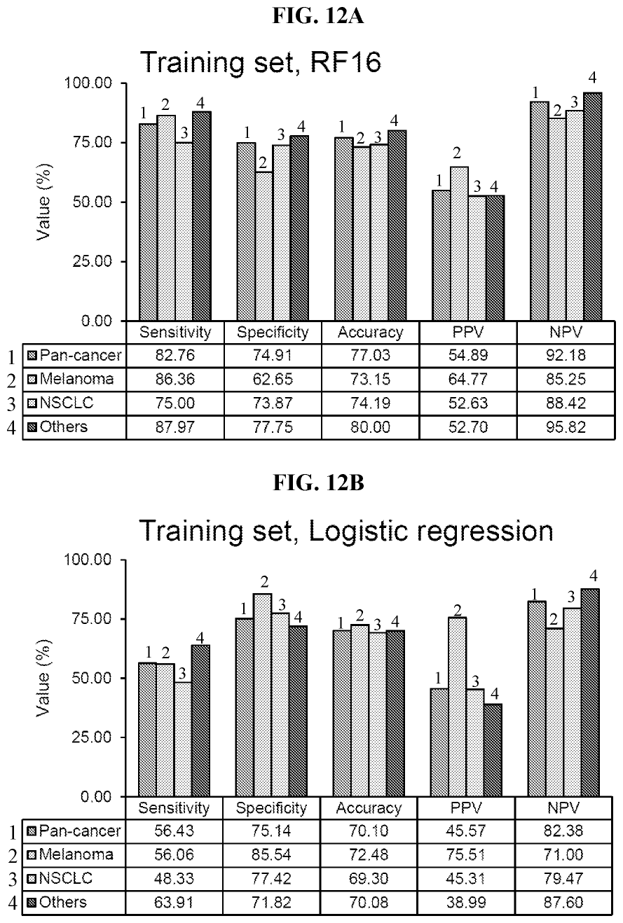

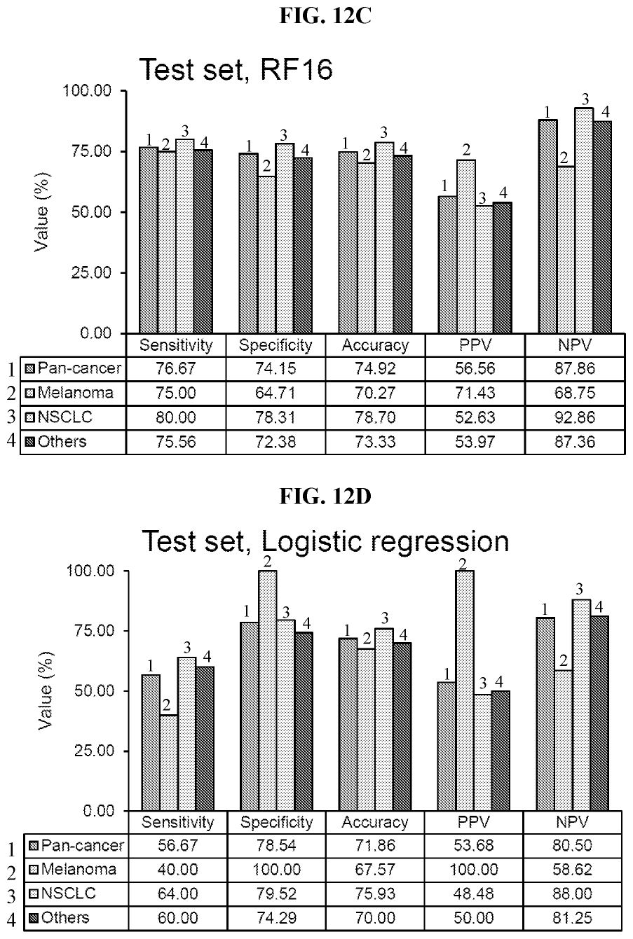

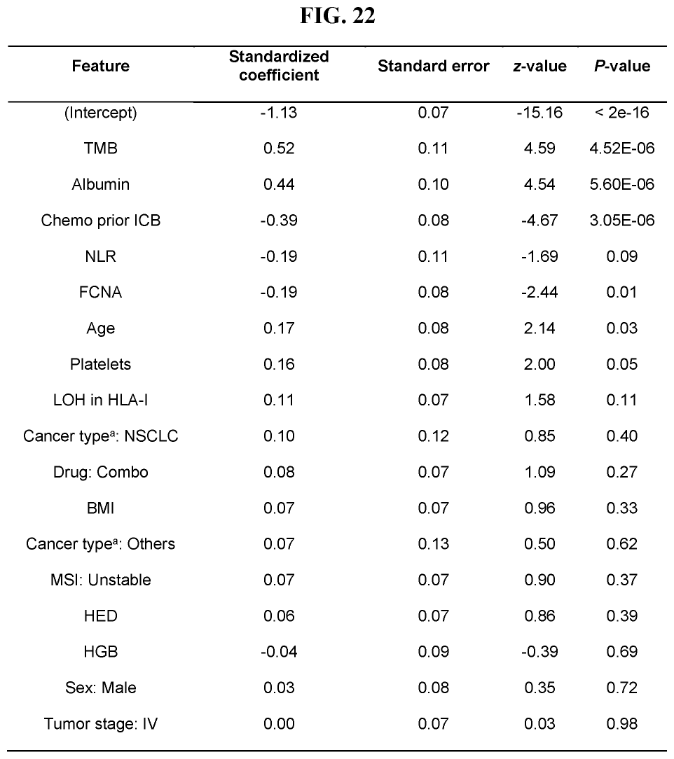

The performance of RF16 was further compared with that of a logistic regression using the same training data and same model features for model calibration (FIG. 22). The RF16 model consistently achieved higher predictive performance compared to the logistic regression in pan-cancer, melanoma, NSCLC, and others, in both the training and test set (FIGS. 12A-12D). This result may be partly explained because RF16 does not assume that the relationships between the model variables and the log odds of immunotherapy response are linear, which logistic regression does assume.

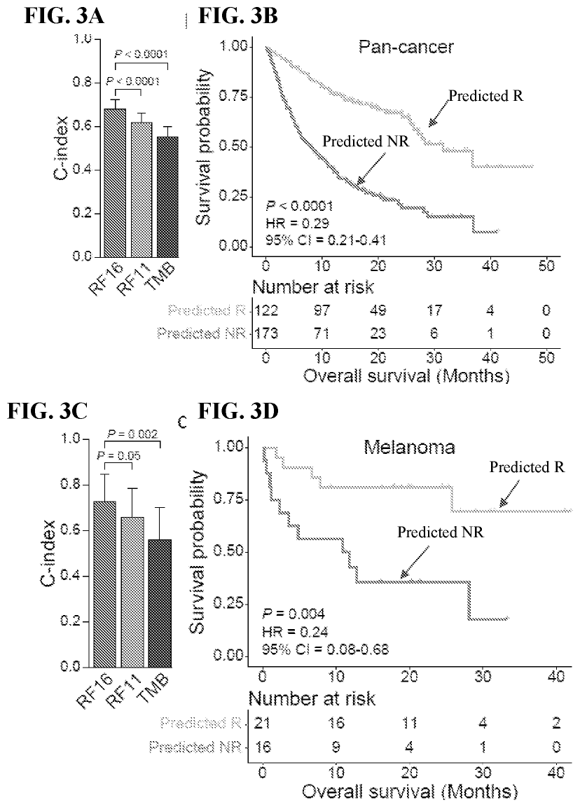

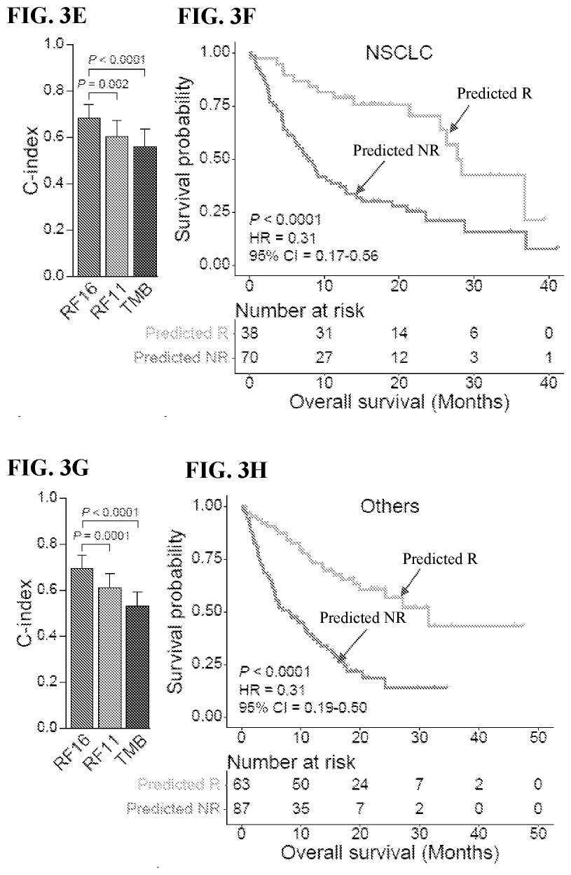

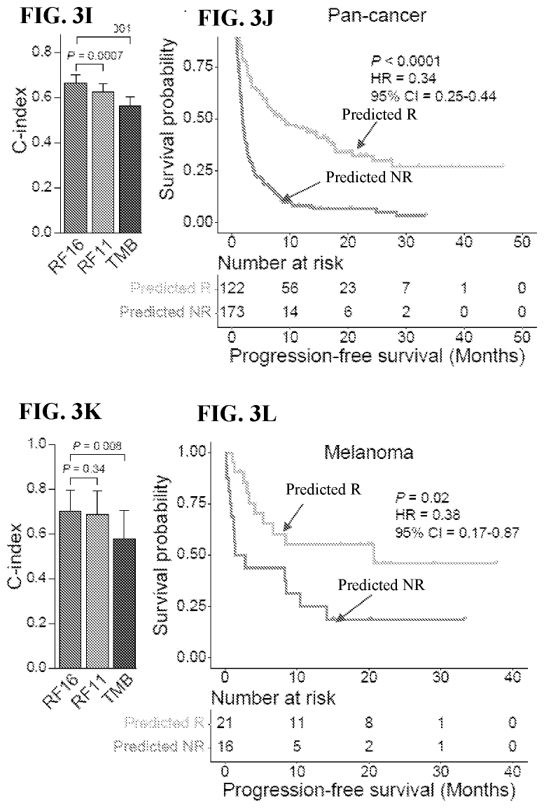

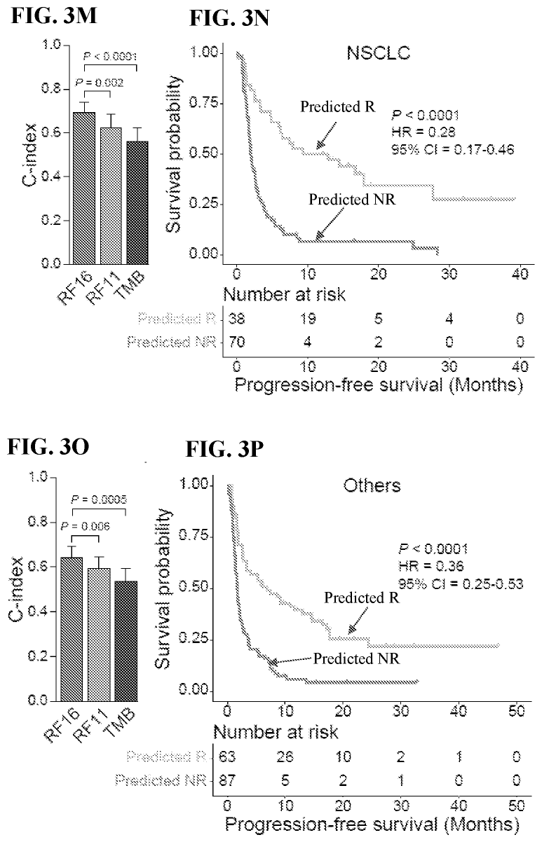

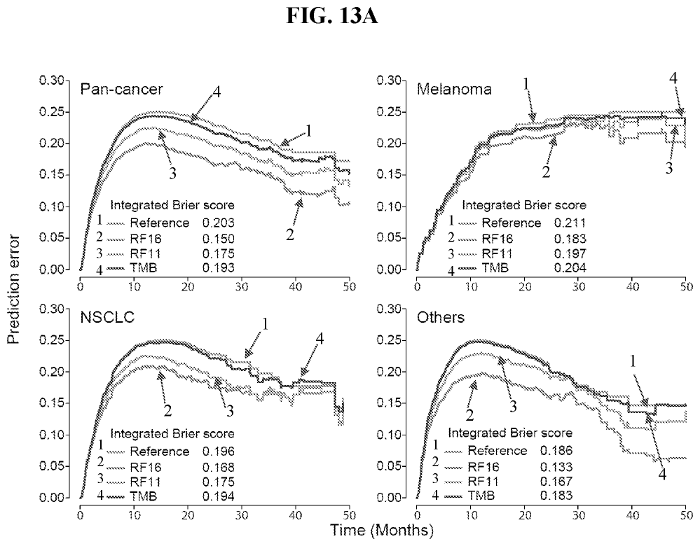

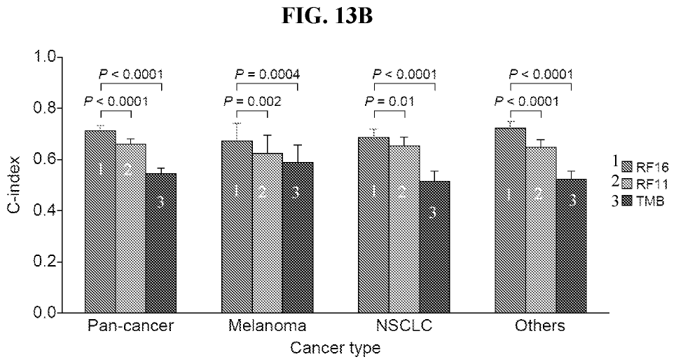

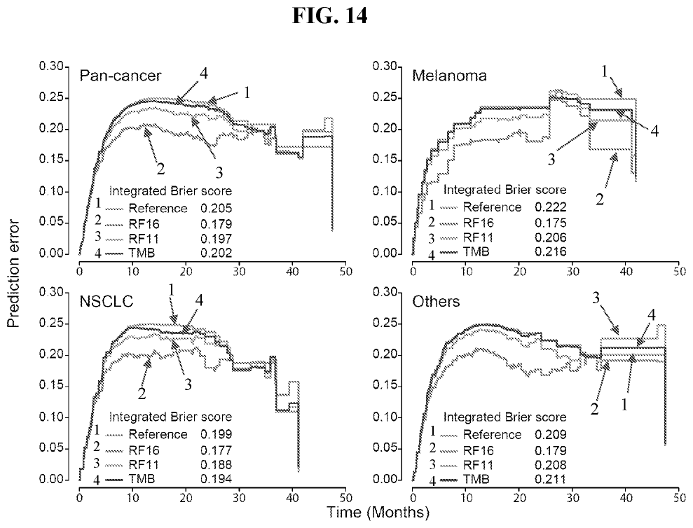

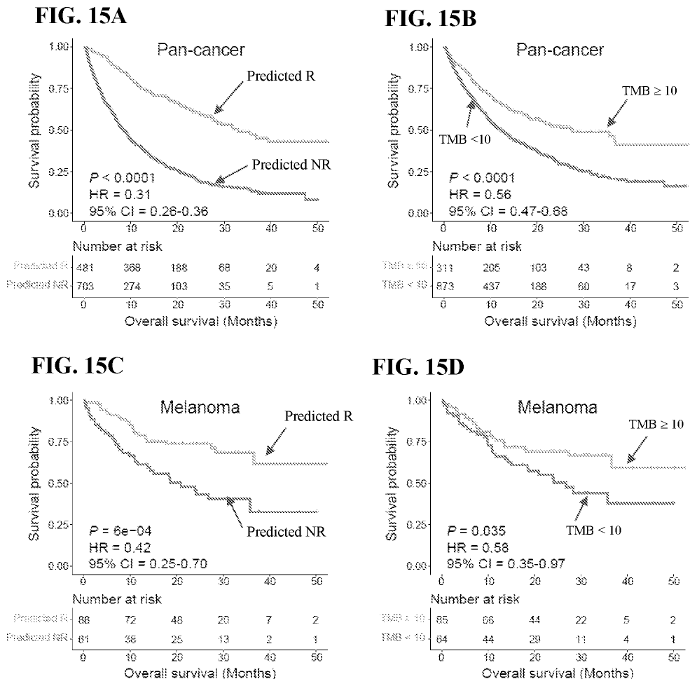

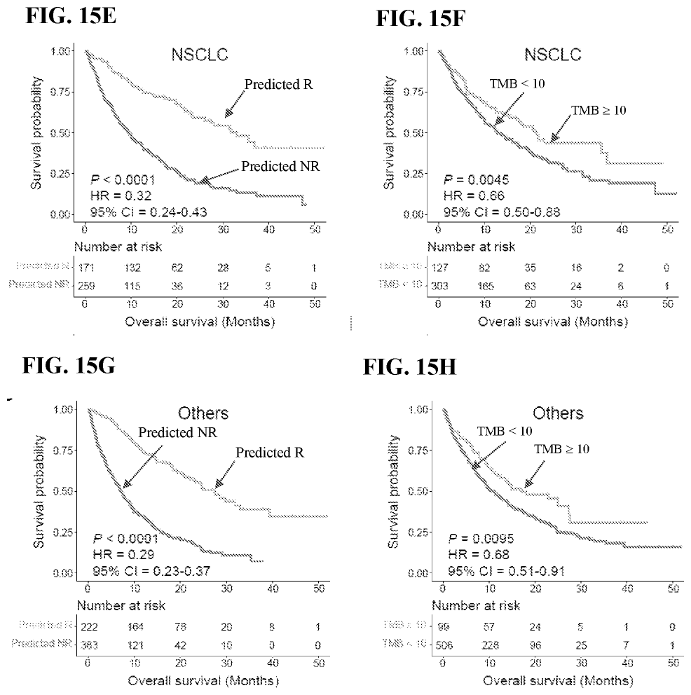

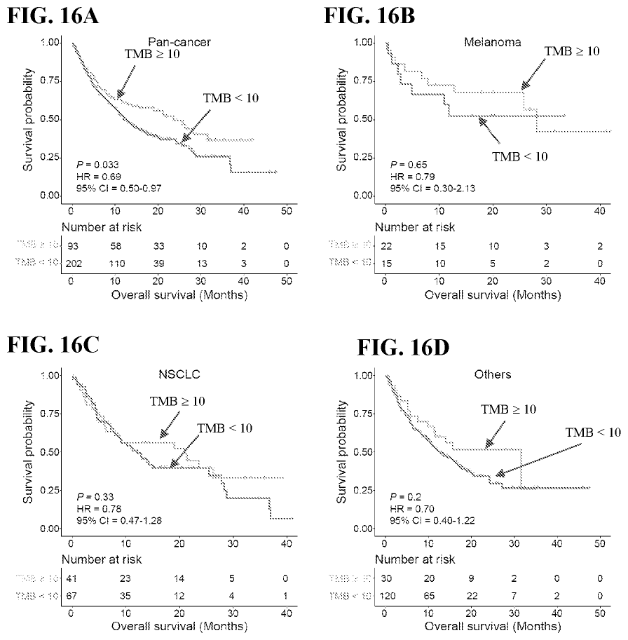

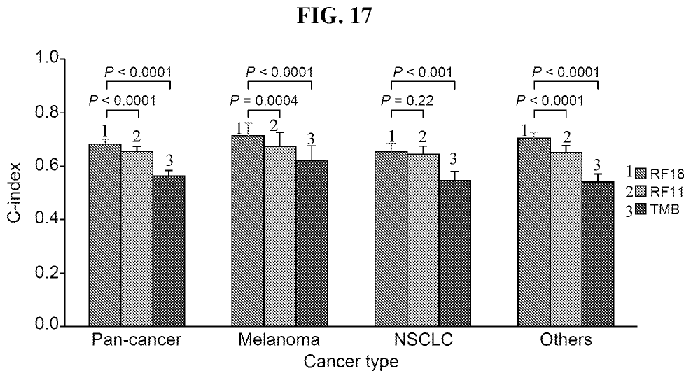

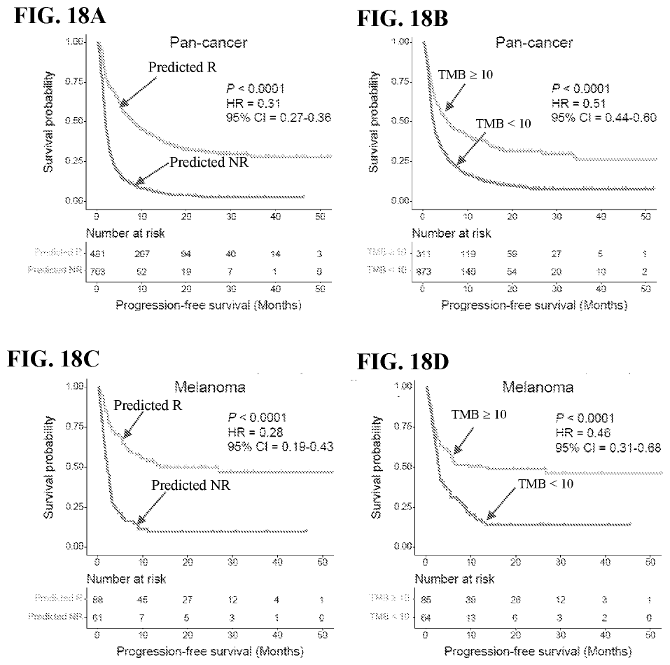

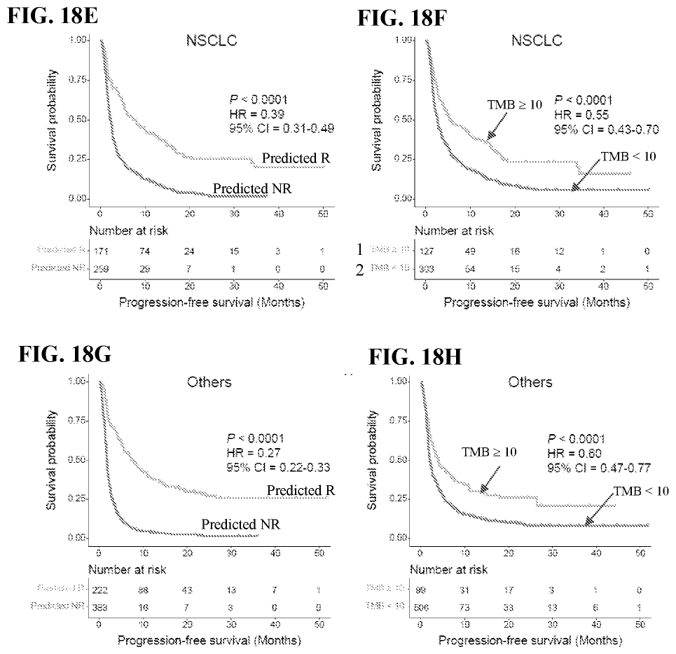

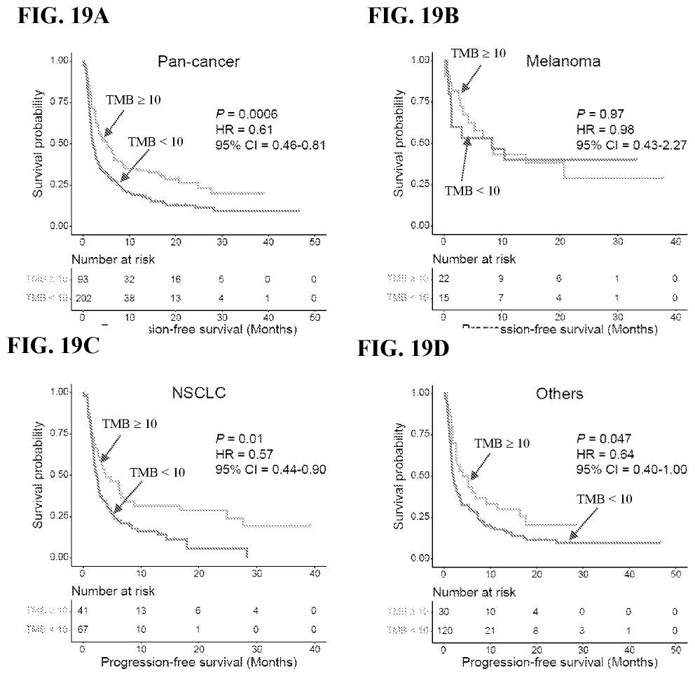

To test whether the model of the present technology could also predict overall survival (OS) before administration of immunotherapy, the brier score, which quantifies the accuracy of a set of predictions by calculating the error between observed and predicted OS probabilities, was used. The RF16 model predictions had a smaller error compared to either a reference (random) model, based on TMB alone, or inferred by RF11 both in the training data and the test data (FIGS. 13A and 14). The concordance index (C-index) for OS, which ranges between 0 and 1 (0.5 being random performance) was further calculated. It was found that the C-indices of the RF16 predictions were significantly higher than those generated by TMB or RF11 across tumor types, both in the training set (FIG. 13B; pan-cancer C-index 0.71 for RF16 versus 0.66 for RF11 versus 0.54 for TMB, P<0.0001) and the test set (FIGS. 3A, 3C, 3E, and 3G; pan-cancer C-index 0.68 for RF16 versus 0.62 for RF11 versus 0.55 for TMB, P<0.0001). Additionally, it was found that responders predicted by the RF16 model were significantly associated with longer OS compared to patients classified as non-responders in the training set (FIGS. 15A, 15C, 15E, and 15G; pan-cancer P<0.0001, hazard ratio (HR)=0.31, 95% confidence interval (CI)=0.26-0.36) and the test set (FIGS. 3B, 3D, 3F and 311; pan-cancer P<0.0001, HR=0.29, 95% CI=0.21-0.41). Furthermore, the differences in OS between responders and non-responders predicted by RF16 were significantly higher compared to differences between responder and non-responder groups predicted by TMB alone across the various cancer types (FIGS. 3B, 3D, 3F and 311, FIGS. 15A-15H and 16A-16D). Additionally, the predictions of progression-free survival (PFS) produced by RF16 were significantly more accurate than both TMB alone and based on RF11 in the training set (FIG. 17; pan-cancer C-index 0.68 for RF16 versus 0.66 for RF11 versus 0.56 for TMB, P<0.0001) and the test set (FIGS. 31, 3K, 3M, and 3O; pan-cancer C-index 0.67 for RF16 versus 0.62 for RF11 versus 0.56 for TMB, P=0.0007 and P<0.0001, respectively). Consistent with these results, responders predicted by the RF16 model also had significantly better PFS than predicted non-responders in the training (FIGS. 18A, 18C, 18E, and 18G; pan-cancer P<0.0001, HR=0.31, 95% CI=0.27-0.36) and the test data (FIGS. 3J, 3L, 3N, and 3P; pan-cancer P<0.0001, HR=0.34, 95% CI=0.25-0.44) with larger PFS differences between responders and non-responders predicted by RF16 than TMB alone across the various cancer types (FIGS. 3J, 3L, 3N, and 3P, and FIGS. 18A-18H and 19A-19D).

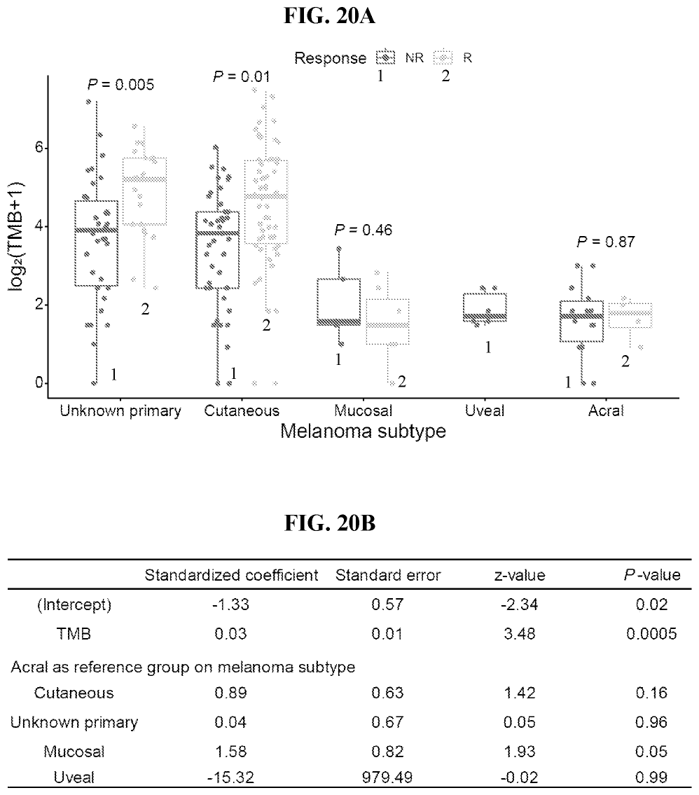

Altogether, these data demonstrate that the machine learning approach disclosed herein can forecast response, OS, and PFS prior to administration of immunotherapy at high accuracy. In addition, these results demonstrate that accurate prediction of ICB response required an integrated model incorporating genetic (both germline and somatic), clinical and demographic factors, and blood markers suggestive of the overall health of the patient. Importantly, each of the model features can be easily measured from blood and from tumor tissue DNA sequencing. Additionally, the values of the peripheral blood markers used in the model such as NLR, albumin, platelets, and HGB are routinely performed in almost all blood tests in the clinic. Additionally, these results revealed that multiple biological factors independently contributed, with various degrees of effects, to response. In contrast to what it has been suggested (Gurjao, C., Tsukrov, D., Imakaev, M., Luquette, L. J. & Mirny, L. bioRxiv, 2020.2009.2003.260265), the association of TMB with immunotherapy response was not confounded by the melanoma subtype (FIG. 20).

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

### REFERENCES

1 Topalian, S. L., Taube, J. M., Anders, R. A. & Pardoll, D. M. Mechanism-driven biomarkers to guide immune checkpoint blockade in cancer therapy. Nat Rev Cancer 16, 275-287, doi:10.1038/nrc.2016.36 (2016).
2 Havel, J. J., Chowell, D. & Chan, T. A. The evolving landscape of biomarkers for checkpoint inhibitor immunotherapy. Nat Rev Cancer 19, 133-150, doi:10.1038/s41568-019-0116-x (2019).
3 Prasad, V., De Jesus, K. & Mailankody, S. The high price of anticancer drugs: origins, implications, barriers, solutions. Nat Rev Clin Oncol 14, 381-390, doi:10.1038/nrclinonc.2017.31 (2017).
4 Keenan, T. E., Burke, K. P. & Van Allen, E. M. Genomic correlates of response to immune checkpoint blockade. Nat Med 25, 389-402, doi:10.1038/s41591-019-0382-x (2019).
5 Subbiah, V., Solit, D. B., Chan, T. A. & Kurzrock, R. The FDA approval of pembrolizumab for adult and pediatric patients with tumor mutational burden (TMB) >/=10: a decision centered on empowering patients and their physicians. Ann Oncol 31, 1115-1118, doi:10.1016/j.annonc.2020.07.002 (2020).
6 Bendell, J. et al. Efficacy and safety results from IMblaze370, a randomised Phase III study comparing atezolizumab plus cobimetinib and atezolizumab monotherapy vs regorafenib in chemotherapy-refractory metastatic colorectal cancer. Annals of Oncology 29, 123-123 (2018).
7 Carbone, D. P. et al. First-Line Nivolumab in Stage IV or Recurrent Non-Small-Cell Lung Cancer. N Engl J Med 376, 2415-2426, doi:10.1056/NEJMoa1613493 (2017).
8 Cohen, E. E. et al. Pembrolizumab (pembro) vs standard of care (SOC) for recurrent or metastatic head and neck squamous cell carcinoma (R/M HNSCC): Phase 3 KEYNOTE-040 trial. Annals of Oncology 28 (2017).
9 Powles, T. et al. Atezolizumab versus chemotherapy in patients with platinum-treated locally advanced or metastatic urothelial carcinoma (IMvigor211): a multicentre, open-label, phase 3 randomised controlled trial. Lancet 391, 748-757, doi:10.1016/S0140-6736(17)33297-X (2018).
10 Anagnostou, V. et al. Multimodal genomic features predict outcome of immune checkpoint blockade in non-small-cell lung cancer. Nat Cancer 1, 99-111, doi:10.1038/s43018-019-0008-8 (2020).
11 Topol, E. J. High-performance medicine: the convergence of human and artificial intelligence. Nat Med 25, 44-56, doi:10.1038/s41591-018-0300-7 (2019).
12 Rajkomar, A., Dean, J. & Kohane, I. Machine Learning in Medicine. N Engl J Med 380, 1347-1358, doi:10.1056/NEJMra1814259 (2019).
13 Eisenhauer, E. A. & Verweij, J. New response evaluation criteria in solid tumors: RECIST GUIDELINE VERSION 1.1. Ejc Suppl 7, 5-5, doi:Doi 10.1016/S1359-6349(09)70018-7 (2009).
14 Zehir, A. et al. Mutational landscape of metastatic cancer revealed from prospective clinical sequencing of 10,000 patients. Nat Med 23, 703-713, doi:10.1038/nm.4333 (2017).
15 Breiman, L. Random forests. Mach Learn 45, 5-32, doi:Doi 10.1023/A:1010933404324 (2001).
16 Rizvi, N. A. et al. Cancer immunology. Mutational landscape determines sensitivity to PD-1 blockade in non-small cell lung cancer. Science 348, 124-128, doi:10.1126/science.aaa1348 (2015).
17 Snyder, A. et al. Genetic basis for clinical response to CTLA-4 blockade in melanoma. N Engl J Med 371, 2189-2199, doi:10.1056/NEJMoa1406498 (2014).
18 Van Allen, E. M. et al. Genomic correlates of response to CTLA-4 blockade in metastatic melanoma. Science 350, 207-211, doi:10.1126/science.aad0095 (2015).
19 Riaz, N. et al. Tumor and Microenvironment Evolution during Immunotherapy with Nivolumab. Cell, doi:10.1016/j.cell.2017.09.028 (2017).
20 Goodman, A. M. et al. Tumor Mutational Burden as an Independent Predictor of Response to Immunotherapy in Diverse Cancers. Molecular Cancer Therapeutics 16, 2598-2608, doi:10.1158/1535-7163.Mct-17-0386 (2017).
21 Samstein, R. M. et al. Tumor mutational load predicts survival after immunotherapy across multiple cancer types. Nature Genetics 51, 202−+, doi:10.1038/s41588-018-0312-8 (2019).
22 Luksza, M. et al. A neoantigen fitness model predicts tumour response to checkpoint blockade immunotherapy. Nature 551, 517-520, doi:10.1038/nature24473 (2017).
23 Valero, C. et al. The association between tumor mutational burden and prognosis is dependent on treatment context. Nat Genet 53, 11-15, doi:10.1038/s41588-020-00752-4 (2021).
24 Davoli, T., Uno, H., Wooten, E. C. & Elledge, S. J. Tumor aneuploidy correlates with markers of immune evasion and with reduced response to immunotherapy. Science 355, doi:10.1126/science.aaf8399 (2017).
25 Chowell, D. et al. Evolutionary divergence of HLA class I genotype impacts efficacy of cancer immunotherapy. Nat Med 25, 1715-1720, doi:10.1038/s41591-019-0639-4 (2019).
26 Chowell, D. et al. Patient HLA class I genotype influences cancer response to checkpoint blockade immunotherapy. Science 359, 582-587, doi:10.1126/science.aao4572 (2018).
27 Mandal, R. et al. Genetic diversity of tumors with mismatch repair deficiency influences anti-PD-1 immunotherapy response. Science 364, 485-491, doi:10.1126/science.aau0447 (2019).
28 Le, D. T. et al. Mismatch-repair deficiency predicts response of solid tumors to PD-1 blockade. Science, doi:10.1126/science.aan6733 (2017).
29 Wang, Z. et al. Paradoxical effects of obesity on T cell function during tumor progression and PD-1 checkpoint blockade. Nat Med 25, 141-151, doi:10.1038/s41591-018-0221-5 (2019).
30 Sanchez, A. et al. Transcriptomic signatures related to the obesity paradox in patients with clear cell renal cell carcinoma: a cohort study. Lancet Oncol 21, 283-293, doi:10.1016/S1470-2045(19)30797-1 (2020).
31 Conforti, F. et al. Cancer immunotherapy efficacy and patients' sex: a systematic review and meta-analysis. Lancet Oncol 19, 737-746, doi:10.1016/S1470-2045(18)30261-4 (2018).
32 Jaillon, S. et al. Neutrophil diversity and plasticity in tumour progression and therapy. Nat Rev Cancer 20, 485-503, doi:10.1038/s41568-020-0281-y (2020).
33 Li, M. J. et al. Change in neutrophil to lymphocyte ratio during immunotherapy treatment is a non-linear predictor of patient outcomes in advanced cancers. J Cancer Res Clin 145, 2541-2546, doi:10.1007/s00432-019-02982-4 (2019).
34 Valero, C. et al. Pretreatment neutrophil-to-lymphocyte ratio and mutational burden as biomarkers of tumor response to immune checkpoint inhibitors. Nat Commun 12, 729, doi:10.1038/s41467-021-20935-9 (2021).
35 Kuai, J., Yang, F., Li, G. J., Fang, X. J. & Gao, B. Q. In vitro-activated tumor-specific T lymphocytes prolong the survival of patients with advanced gastric cancer: a retrospective cohort study. Onco Targets Ther 9, 3763-3770, doi:10.2147/OTT.S102909 (2016).
36 Ikeguchi, A., Machiorlatti, M. & Vesely, S. K. Disparity in outcomes of melanoma adjuvant immunotherapy by demographic profile. Melanoma Manag 7, MMT43, doi:10.2217/mmt-2020-0002 (2020).
37 Jurasz, P., Alonso-Escolano, D. & Radomski, M. W. Platelet—cancer interactions: mechanisms and pharmacology of tumour cell-induced platelet aggregation. Br J Pharmacol 143, 819-826, doi:10.1038/sj.bjp.0706013 (2004).
38 Gupta, D. & Lis, C. G. Pretreatment serum albumin as a predictor of cancer survival: A systematic review of the epidemiological literature. Nutr J 9, doi:Artn 69 10.1186/1475-2891-9-69 (2010).
39 Caro, J. J., Salas, M., Ward, A. & Goss, G. Anemia as an independent prognostic factor for survival in patients with cancer—A systematic, quantitative review. Cancer 91, 2214-2221, doi:Doi 10.1002/1097-0142(20010615)91:12<2214::Aid-Cncrl251>3.3.Co; 2-G (2001).
40 Middha, S. et al. Reliable Pan-Cancer Microsatellite Instability Assessment by Using Targeted Next-Generation Sequencing Data. Jco Precis Oncol 1, doi:10.1200/Po.17.00084 (2017).
41 Hanahan, D. & Weinberg, R. A. Hallmarks of cancer: the next generation. Cell 144, 646-674, doi:10.1016/j.cell.2011.02.013 (2011).
42 Peng, D. et al. Prognostic significance of HALP (hemoglobin, albumin, lymphocyte and platelet) in patients with bladder cancer after radical cystectomy. Sci Rep 8, 794, doi:10.1038/s41598-018-19146-y (2018).
43 Bindea, G., Mlecnik, B., Fridman, W. H., Pages, F. & Galon, J. Natural immunity to cancer in humans. Curr Opin Immunol 22, 215-222, doi:10.1016/j.coi.2010.02.006 (2010).
44 Harrell, F. E., Jr., Lee, K. L. & Mark, D. B. Multivariable prognostic models: issues in developing models, evaluating assumptions and adequacy, and measuring and reducing errors. Statistics in medicine 15, 361-387, doi:10.1002/(SICI)1097-0258(19960229)15:4<361::AID-SIM168>3.0.CO; 2-4 (1996).
45 Steyerberg, E. W. et al. Assessing the performance of prediction models: a framework for traditional and novel measures. Epidemiology 21, 128-138, doi:10.1097/EDE.0b013e3181c30fb2 (2010).
46 Gurjao, C., Tsukrov, D., Imakaev, M., Luquette, L. J. & Mirny, L. A. Limited evidence of tumour mutational burden as a biomarker of response to immunotherapy. bioRxiv, 2020.2009.2003.260265, doi:10.1101/2020.09.03.260265 (2020).
47 Binnewies, M. et al. Understanding the tumor immune microenvironment (TIME) for effective therapy. Nat Med 24, 541-550, doi:10.1038/s41591-018-0014-x (2018).
48 Gopalakrishnan, V. et al. Gut microbiome modulates response to anti-PD-1 immunotherapy in melanoma patients. Science 359, 97-103, doi:10.1126/science.aan4236 (2018).
49 Krishna, C., Chowell, D., Gonen, M., Elhanati, Y. & Chan, T. A. Genetic and environmental determinants of human TCR repertoire diversity. Immun Ageing 17, 26, doi:10.1186/s12979-020-00195-9 (2020).
50 Braun, D. A. et al. Interplay of somatic alterations and immune infiltration modulates response to PD-1 blockade in advanced clear cell renal cell carcinoma. Nat Med 26, 909-918, doi:10.1038/s41591-020-0839-y (2020).
51 Miao, D. et al. Genomic correlates of response to immune checkpoint blockade in microsatellite-stable solid tumors. Nat Genet 50, 1271-1281, doi:10.1038/s41588-018-0200-2 (2018).
52 Patel, S. J. et al. Identification of essential genes for cancer immunotherapy. Nature 548, 537-542, doi:10.1038/nature23477 (2017).
53 Zaretsky, J. M. et al. Mutations Associated with Acquired Resistance to PD-1 Blockade in Melanoma. N Engl J Med 375, 819-829, doi:10.1056/NEJMoaI604958 (2016).
54 Skoulidis, F. et al. STK11/LKB1 Mutations and PD-1 Inhibitor Resistance in KRAS-Mutant Lung Adenocarcinoma. Cancer Discov 8, 822-835, doi:10.1158/2159-8290.CD-18-0099 (2018).
55 Samstein, R. M. et al. Mutations in BRCA1 and BRCA2 differentially affect the tumor microenvironment and response to checkpoint blockade immunotherapy. Nature Cancer 1, 1188-1203, doi:10.1038/s43018-020-00139-8 (2020).
56 Wang, F. et al. Evaluation of POLE and POLD1 Mutations as Biomarkers for Immunotherapy Outcomes Across Multiple Cancer Types. JAMA Oncology 5, 1504-1506, doi:10.1001/jamaoncol.2019.2963 (2019).
57 Amin, M. B. et al. The Eighth Edition AJCC Cancer Staging Manual: Continuing to build a bridge from a population-based to a more “personalized” approach to cancer staging. CA Cancer J Clin 67, 93-99, doi:10.3322/caac.21388 (2017).
58 Zhou, J. et al. Analysis of Tumor Genomic Pathway Alterations Using Broad-Panel Next-Generation Sequencing in Surgically Resected Lung Adenocarcinoma. Clinical Cancer Research 25, 7475-7484, doi:10.1158/1078-0432.Ccr-19-1651 (2019).
59 Shen, R. L. & Seshan, V. E. FACETS: allele-specific copy number and clonal heterogeneity analysis tool for high-throughput DNA sequencing. Nucleic Acids Research 44, doi:ARTN e131 10.1093/nar/gkw520 (2016).
60 Niu, B. F. et al. MSIsensor: microsatellite instability detection using paired tumor-normal sequence data. Bioinformatics 30, 1015-1016, doi:10.1093/bioinformatics/btt755 (2014).
61 Shukla, S. A. et al. Comprehensive analysis of cancer-associated somatic mutations in class I HLA genes. Nat Biotechnol 33, 1152-1158, doi:10.1038/nbt.3344 (2015).
62 Quinlan, A. R. & Hall, I. M. BEDTools: a flexible suite of utilities for comparing genomic features. Bioinformatics 26, 841-842, doi:10.1093/bioinformatics/btq033 (2010).
63 Pierini, F. & Lenz, T. L. Divergent allele advantage at human MHC genes: signatures of past and ongoing selection. Mol Biol Evol, doi:10.1093/molbev/msy116 (2018).
64 Robinson, J. et al. The IPD and IMGT/HLA database: allele variant databases. Nucleic Acids Research 43, D423-D431, doi:10.1093/nar/gku1161 (2015).
65 Zerbino, D. R. et al. Ensembl 2018. Nucleic Acids Res 46, D754-D761, doi:10.1093/nar/gkx1098 (2018).
66 Grantham, R. Amino acid difference formula to help explain protein evolution. Science 185, 862-864 (1974).
67 Edgar, R. C. MUSCLE: multiple sequence alignment with high accuracy and high throughput. Nucleic Acids Res 32, 1792-1797, doi:10.1093/nar/gkh340 (2004).
68 Pedregosa, F. et al. Scikit-learn: Machine Learning in Python. J Mach Learn Res 12, 2825-2830 (2011).
69 Robin, X. et al. pROC: an open-source package for R and S+ to analyze and compare ROC curves. BMC Bioinformatics 12, 77, doi:10.1186/1471-2105-12-77 (2011).
70 Schroder, M. S., Culhane, A. C., Quackenbush, J. & Haibe-Kains, B. survcomp: an R/Bioconductor package for performance assessment and comparison of survival models. Bioinformatics 27, 3206-3208, doi:10.1093/bioinformatics/btr511 (2011).
71 Mogensen, U. B., Ishwaran, H. & Gerds, T. A. Evaluating Random Forests for Survival Analysis Using Prediction Error Curves. J Stat Softw 50, 1-23 (2012).
72 Saito, T. & Rehmsmeier, M. Precrec: fast and accurate precision-recall and ROC curve calculations in R. Bioinformatics 33, 145-147, doi:10.1093/bioinformatics/btw570 (2017).

1. A method of training a machine learning classifier for predicting responsiveness of cancer patients to an immune checkpoint blockade (ICB) therapy, the method comprising:
receiving data on a cohort of subjects, the subjects in the cohort having a plurality of cancer types;
generating a training dataset based on the received data, the training dataset comprising a plurality of features for each subject in the cohort, the plurality of features comprising at least one of a blood albumin level, a blood hemoglobin level, or a blood platelet level; and
applying a machine learning method to the training dataset to develop the machine learning classifier for predicting responsiveness of cancer patients to the ICB therapy,
wherein applying the machine learning method comprises:
applying a machine learning technique to the training dataset to each cancer type individually;
performing hyperparameter optimization to identify one or more machine learning models with an accuracy that exceeds an accuracy threshold for the classifier; and
determining an optimal operating-point threshold based on optimization of sensitivity and specificity of the receiver operating characteristic (ROC) curves for the training dataset; and
wherein the classifier is configured to receive the plurality of features for cancer patients and generate predictors for responsiveness of the cancer patients to the ICB therapy.

2.-19. (canceled)

20. A method of predicting responsiveness of a cancer patient to an immune checkpoint blockade (ICB) therapy using a machine learning classifier, the method comprising:
receiving patient data corresponding to a plurality of features for the cancer patient;
applying the machine learning classifier to the patient data to generate a predictor; and
determining whether the cancer patient is predicted to be responsive to the ICB therapy based on the predictor and an operating-point threshold, wherein the machine learning classifier is trained by:
receiving cohort data on a cohort of subjects, the subjects in the cohort having a plurality of cancer types;
generating a training dataset based on the received cohort data, the training dataset comprising the plurality of features for each subject in the cohort, the plurality of features comprising at least one of a blood albumin level, a blood hemoglobin level, or a blood platelet level; and
applying a machine learning method to the training dataset to develop the machine learning classifier for predicting responsiveness of cancer patients to the ICB therapy,
wherein applying the machine learning method comprises:
applying a machine learning technique to the training dataset to each cancer type individually;
performing hyperparameter optimization to identify one or more machine learning models with an accuracy that exceeds an accuracy threshold for the machine learning classifier; and
determining the optimal operating-point threshold based on optimization of sensitivity and specificity of the receiver operating characteristic (ROC) curves for the training dataset; and
wherein the machine learning classifier is configured to receive the plurality of features for cancer patients and generate predictors for responsiveness of the cancer patients to the ICB therapy.

21. The method of claim 20, further comprising administering an effective amount of the ICB therapy to the cancer patient predicted to be responsive to the ICB therapy based on the predictor and the operating-point threshold, optionally wherein the predictor comprises a response probability value.

22. (canceled)

23. The method of claim 20, wherein the machine learning technique is a random forest technique, and wherein the one or more machine learning models are random forest models.

24. The method of claim 20, wherein the machine learning classifier is an ensemble learning random forest classifier or wherein the machine learning classifier is cancer-type specific, and wherein the plurality of features comprises a cancer type.

25. (canceled)

26. The method of claim 20, wherein hyperparameter optimization is performed for each cancer type in the plurality of cancer types or wherein performing the hyperparameter optimization comprises performing an exhaustive grid search technique.

27. (canceled)

28. The method of claim 20, wherein the plurality of features comprises
a plurality of at least one genomic feature, at least one molecular feature, at least one clinical feature, and/or at least one demographic feature; or
at least one genomic feature, at least one molecular feature, at least one clinical feature, and at least one demographic feature.

29. (canceled)

30. The method of claim 20, wherein the plurality of features comprises
a plurality of: (i) a tumor mutation burden (TMB) metric, (ii) a fraction of copy-number alteration (FCNA) metric, (iii) an HLA-I evolutionary divergence (HED) metric, (iv) a loss of heterozygosity (LOH) status in HLA-I, (v) a microsatellite instability (MSI) status, (vi) a body mass index (BMI), (vii) a gender, (viii) a blood neutrophil-to-lymphocyte ratio (NLR) metric, (ix) a tumor stage, (x) an immune checkpoint inhibitor, (xi) an age, (xii) a cancer type, and/or (xiii) an indication of whether chemotherapy has been administered; or
(i) a tumor mutation burden (TMB) metric, (ii) a fraction of copy-number alteration (FCNA) metric, (iii) an HLA-I evolutionary divergence (HED) metric, (iv) a loss of heterozygosity (LOH) status in HLA-I, (v) a microsatellite instability (MSI) status, (vi) a body mass index (BMI), (vii) a gender, (viii) a blood neutrophil-to-lymphocyte ratio (NLR) metric, (ix) a tumor stage, (x) an immune checkpoint inhibitor, (xi) an age, (xii) a cancer type, (xiii) an indication of whether chemotherapy has been administered, (xiv) the blood albumin level, (xv) the blood hemoglobin level, and (xvi) the blood platelet level.

31. (canceled)

32. The method of claim 20, wherein the plurality of features comprises one or more features having discrete values and/or one or more features having continuous values.

33. The method of claim 32, wherein the one or more features having discrete values are selected from among an indication of whether chemotherapy has been administered, cancer type, LOH in HLA-I, an immune checkpoint inhibitor, MSI status, and tumor stage.

34. The method of claim 32, wherein the one or more features having continuous values are selected from among a TMB metric, blood albumin level, blood NLR metric, age, blood hemoglobin level, blood platelet level, a FCNA metric, BMI, and a HED metric.

35. The method of claim 20, wherein the plurality of cancer types are selected from the group consisting of non-small cell lung cancers (NSCLC), small cell lung cancers (SCLC), melanoma, renal cell carcinoma, bladder cancer, head and neck cancer, sarcoma, endometrial cancer, gastric cancer, hepatobiliary cancer, colorectal cancer, esophageal cancer, pancreatic cancer, mesothelioma, ovarian cancer, and breast cancer.

36. The method of claim 20, wherein the ICB therapy is a PD-1/PD-L1 inhibitor, a CTLA-4 inhibitor, or a combination thereof, optionally wherein the ICB therapy comprises one or more of pembrolizumab, nivolumab, cemiplimab, atezolizumab, avelumab, durvalumab, ipilimumab, tremelimumab, ticlimumab, JTX-4014, Spartalizumab (PDR001), Camrelizumab (SHR1210), Sintilimab (IBI308), Tislelizumab (BGB-A317), Toripalimab (JS 001), Dostarlimab (TSR-042, WBP-285), INCMGA00012 (MGA012), AMP-224, AMP-514, KN035, CK-301, AUNP12, CA-170, or BMS-986189.

37. (canceled)

38. The method of claim 21, wherein the cancer patient predicted to be responsive to the ICB therapy based on the predictor and the operating-point threshold exhibits extended overall survival and/or progression-free survival compared to a cancer patient that is predicted to be non-responsive to the ICB therapy based on the predictor and the operating-point threshold.

39. The method of claim 20, wherein the plurality of features for each subject in the cohort are determined by assaying blood and/or sequencing tumor DNA.

40. The method of claim 20, wherein the plurality of features for the cancer patient are determined by assaying blood and/or sequencing tumor DNA.

41. The method of claim 30, wherein the tumor stage is stage I, stage II, Stage III or stage IV.

42. The method of claim 20, wherein the predictors comprise response probability values.

43-60. (canceled)

61. A computing system for predicting responsiveness of a cancer patient to an immune checkpoint blockade (ICB) therapy, the computing system comprising a processor and a memory with instructions which, when executed by the processor, cause the processor to:
receive patient data corresponding to a plurality of features for the cancer patient;
apply a machine learning classifier to the patient data to generate a predictor; and
determine whether the cancer patient is predicted to be responsive to the ICB therapy based on the predictor and an operating-point threshold, wherein the classifier is trained by:
receiving cohort data on a cohort of subjects, the subjects in the cohort having a plurality of cancer types;
generating a training dataset based on the received cohort data, the training dataset comprising the plurality of features for each subject in the cohort, the plurality of features comprising at least one of a blood albumin level, a blood hemoglobin level, or a blood platelet level; and
applying a machine learning method to the training dataset to develop the machine learning classifier for predicting responsiveness of cancer patients to the ICB therapy,
wherein applying the machine learning method comprises:
applying a machine learning technique to the training dataset to each cancer type individually;
performing hyperparameter optimization to identify one or more machine learning models with an accuracy that exceeds an accuracy threshold for the machine learning classifier; and
determining the optimal operating-point threshold based on optimization of sensitivity and specificity of the receiver operating characteristic (ROC) curves for the training dataset; and
wherein the machine learning classifier is configured to receive the plurality of features for cancer patients and generate predictors for responsiveness of the cancer patients to the ICB therapy.

62.-96. (canceled)

97. A non-transitory computer-readable storage medium comprising instructions which, when executed by a processor of a computing system, configure the computing system to system for predicting responsiveness of a cancer patient to an immune checkpoint blockade (ICB) therapy, the instructions configured to cause the processor to:
receive patient data corresponding to a plurality of features for the cancer patient;
apply a machine learning classifier to the patient data to generate a predictor; and
determine whether the cancer patient is predicted to be responsive to the ICB therapy based on the predictor and an operating-point threshold, wherein the classifier is trained by:
receiving cohort data on a cohort of subjects, the subjects in the cohort having a plurality of cancer types;
generating a training dataset based on the received cohort data, the training dataset comprising the plurality of features for each subject in the cohort, the plurality of features comprising at least one of a blood albumin level, a blood hemoglobin level, or a blood platelet level; and
applying a machine learning method to the training dataset to develop the machine learning classifier for predicting responsiveness of cancer patients to the ICB therapy,
wherein applying the machine learning method comprises:
applying a machine learning technique to the training dataset to each cancer type individually;
performing hyperparameter optimization to identify one or more machine learning models with an accuracy that exceeds an accuracy threshold for the machine learning classifier; and
determining the optimal operating-point threshold based on optimization of sensitivity and specificity of the receiver operating characteristic (ROC) curves for the training dataset; and
wherein the machine learning classifier is configured to receive the plurality of features for cancer patients and generate predictors for responsiveness of the cancer patients to the ICB therapy.

98.-114. (canceled)
